(19) United States
(12) Patent Application Publication
(10) Pub. No.: US 2025/0154284 A1
(43) Pub. Date: May 15, 2025

(54) MASP-2-TARGETTING ANTIBODIES AND USES THEREOF

(71) Applicant: INMAGENE PTE. LTD., Singapore (SG)

(72) Inventors: Chongtian GUO, Shanghai (CN); Run LEI, Shanghai (CN); Pengcheng FAN, Zhejiang, Hangzhou (CN); Lihua FAN, Zhejiang, Hangzhou (CN); Zhihao XU, Zhejiang, Hangzhou (CN); Qiang SUN, Zhejiang, Hangzhou (CN); Jonathan Jian WANG, Shanghai (CN)

(21) Appl. No.: 18/869,831

(22) PCT Filed: Feb. 24, 2023
(86) PCT No.: PCT/CN2023/078260

§ 371 Date: Nov. 26, 2024

(30) Foreign Application Priority Data
Mar. 18, 2022 (WO) PCT/CN2022/081803

(51) Int. Cl. C07K 16/40

(52) U.S. Cl. CPC C07K 16/40; C12Y 304/21104; C07K 2317/24; C07K 2317/34; C07K 2317/76; C07K 2317/92

(57) ABSTRACT

Disclosed herein are anti-MASP-2 antibodies and antigen-binding fragments, polynucleotides encoding the antibodies and antigen-binding fragments, and pharmaceutical compositions comprising the antibodies and antigen-binding fragments. Uses of the anti-MASP-2 antibodies and antigen-binding fragments described herein in treatment of conditions and disorders associated completement activation are also disclosed.

The present invention claims the priority of the PCT/CN2022/081803, filed on Mar. 18, 2022, the contents of which are incorporated herein by its entirety.

### 1. FIELD

The present invention relates to molecular biology and immunology. Provided herein include anti-MASP-2-antibodies and uses thereof in treating conditions associated with complement activation.

### 2. BACKGROUND

Mannan-binding lectin-associated serine proteinase 2 (MASP-2), an effector enzyme, is required for activation of the lectin pathway of complement. In addition to its essential role in immune defense, the lectin pathway, when activated in an inappropriate or uncontrolled manner, contributes to tissue damage in many clinical conditions. However, there has been limited success in the development of therapeutically effective complement inhibitors that target the lectin pathway. Accordingly, there is an unmet need for additional therapeutic options for MASP-2-targeting agents. The compositions and methods provided herein meet these needs and provide relative advantages.

### 3. SUMMARY

Provided herein are antibodies or antigen-binding fragments thereof that specifically bind human MASP-2, comprising: (1) as defined by Kabat, (a) a light chain variable region (VL) comprising VL CDR1, VL CDR2, VL CDR3 having the amino acid sequences of SEQ ID NOs: 7, 9, and 11, respectively; or a variant thereof having up to about 5 amino acid substitutions, additions, and/or deletions in the VL CDRs; and/or (b) a heavy chain variable region (VH) comprising VH CDR1, VH CDR2, VH CDR3 having the amino acid sequences of SEQ ID NOs: 12, 16, and 18, respectively; or a variant thereof having up to about 5 amino acid substitutions, additions, and/or deletions in the VH CDRs; or (2) as defined by IMGT, (a) a VL comprising VL CDR1, VL CDR2, VL CDR3 having the amino acid sequences of SEQ ID NOs: 8, 10, and 11, respectively; or a variant thereof having up to about 5 amino acid substitutions, additions, and/or deletions in the VL CDRs; and/or (b) a VH comprising VH CDR1, VH CDR2, VH CDR3 having the amino acid sequences of SEQ ID NOs: 13, 17, and 19, respectively; or a variant thereof having up to about 5 amino acid substitutions, additions, and/or deletions in the VH CDRs.

In some embodiments, the antibodies or antigen-binding fragments provided herein comprise VL CDR1, VL CDR2, VL CDR3, VH CDR1, VH CDR2 and VH CDR3 having the amino acid sequences of SEQ ID NOs: 7, 9, 11, 12, 16 and 18, respectively, as defined by Kabat.

In some embodiments, the antibodies or antigen-binding fragments provided herein comprise VL CDR1 having the amino acid sequence of SEQ ID NO: 8; VL CDR2 having the amino acid sequence of SEQ ID NO: 10; VL CDR3 having the amino acid sequence SEQ ID NO: 11; VH CDR1 having the amino acid sequence of SEQ ID NO: 13, 14, or 15; VH CDR2 having the amino acid sequence of SEQ ID NO: 17; and VH CDR3 having the amino acid sequences of SEQ ID NO: 19 or 20; as defined by IMGT.

In some embodiments, the antibodies or antigen-binding fragments provided herein comprise VL CDR1, VL CDR2, VL CDR3, VH CDR1, VH CDR2 and VH CDR3 having the amino acid sequences of: (1) SEQ ID NOs: 8, 10, 11, 13, 17, and 19, respectively; (2) SEQ ID NOs: 8, 10, 11, 14, 17, and 19, respectively; (3) SEQ ID NOs: 8, 10, 11, 15, 17, and 19, respectively; (4) SEQ ID NOs: 8, 10, 11, 13, 17, and 20, respectively; (5) SEQ ID NOs: 8, 10, 11, 14, 17, and 20, respectively; or, (6) SEQ ID NOs: 8, 10, 11, 15, 17, and 20, respectively.

Also provided herein are antibodies or antigen-binding fragments thereof that specifically bind human MASP-2, comprising: (a) a VL having at least 85%, at least 90%, at least 95%, at least 98%, or 100% sequence identity to the amino acid sequence of SEQ ID NO: 21; and/or (b) a VH having at least 85%, at least 90%, at least 95%, at least 98%, or 100% sequence identity to the amino acid sequence of SEQ ID NO: 22.

In some embodiments, the antibodies or antigen-binding fragments provided herein comprise a VL and a VH having the amino acid sequences of SEQ ID NOs: 21 and 22, respectively.

Also provided herein are antibodies or antigen-binding fragments thereof that specifically bind human MASP-2, comprising (a) a VL comprising VL CDR1, VL CDR2, and VL CDR3 from a VL having the amino acid sequence of SEQ ID NO: 21; and/or (b) a VH comprising VH CDR1, VH CDR2, and VH CDR3 from a VH having the amino acid sequence of SEQ ID NO: 22.

In some embodiments, the antibodies or antigen-binding fragments provided herein are chimeric antibodies or antigen-binding fragments, humanized antibodies or antigen-binding fragments, or human antibodies or antigen-binding fragments.

In some embodiments, the antibodies or antigen-binding fragments provided herein are humanized antibodies or antigen-binding fragments. In some embodiments, the antibodies or antigen-binding fragments provided herein comprise: (a) a VL having at least 85%, at least 90%, at least 95%, at least 98%, or 100% sequence identity to the amino acid sequence of SEQ ID NO: 23; and/or (b) a VH having at least 85%, at least 90%, at least 95%, at least 98%, or 100% sequence identity to the amino acid sequence of SEQ ID NO: 24. In some embodiments, the antibodies or antigen-binding fragments provided herein comprise a VL having the amino acid sequence of SEQ ID NO: 23 and a VH having an amino acid sequence selected from the group consisting of SEQ ID NOs: 24-32.

In some embodiments, the antibodies or antigen-binding fragments provided herein are selected from the group consisting of a Fab, a Fab′, a F(ab′)₂, a Fv, a scFv, a (scFv)₂, a single domain antibody (sdAb), and a heavy chain antibody (HCAb).

In some embodiments, the antibodies or antigen-binding fragments provided herein are IgG1 antibodies or variants thereof, IgG2 antibodies or variants thereof, IgG3 antibodies or variants thereof, or IgG4 antibodies or variants thereof.

In some embodiments, provided herein are IgG4 antibodies or variants thereof. In some embodiments, the IgG4 antibodies or variants provided herein comprise (1) a light chain having at least 85%, at least 90%, at least 95%, at least 98%, or 100% sequence identity to the amino acid sequence of SEQ ID NO: 33; and (2) a heavy chain having at least 85%, at least 90%, at least 95%, at least 98%, or 100% sequence identity to an amino acid sequence selected from the group consisting of SEQ ID NO: 34.

In some embodiments, the heavy chain of the antibodies provided herein has an amino acid sequence selected from the group consisting of SEQ ID NOs: 34-42, or a variant thereof modified by one or more amino acid substitution(s) that increases the antibody's terminal half-life. In some embodiments, the heavy chain variant is modified by one or more substitution(s) at an amino acid residue selected from the group consisting of S228, F234, L235, M252, S254, T256, K288, T307, M428, N434, H435, and Y436 (numbered according to the EU Index). In some embodiments, the heavy chain variant is modified by amino acid substitutions selected from the group consisting of i) S228P; ii) F234A and L235A; iii) S228P, F234A, and L235A; (iv) T307H and N434A; v) M252Y, S254T and T256E; vi) M428L, N434A and Y436T; vii) S228P, M252Y, S254T and T256E; viii) S228P, F234A, L235A, M252Y, S254T and T256E; ix) S228P, F234A, L235A, T307Q, and N434A; and (x) M252Y, S254T, T307H and N434A.

In some embodiments of the antibodies provided herein, the light chain has the amino acid sequence of SEQ ID NO: 33 and the heavy chain has an amino acid sequence selected from the group consisting of SEQ ID NOs: 43-74.

Provided herein are also antibodies or antigen-binding fragments thereof that compete with an antibody or antigen-binding fragment described herein for binding to human MASP-2.

In some embodiments, the antibodies or antigen-binding fragments provided herein are bispecific antibodies or multispecific antibodies.

In some embodiments, the antibodies or antigen-binding fragments provided herein are monoclonal antibodies or antigen-binding fragments.

In some embodiments, the antibodies or antigen-binding fragments provided herein: (1) bind human MASP-2 with a K_{D} that is about 1 nM or less, measured by surface plasmon resonance (SPR); (2) block C4 activation with an IC₅₀ of about 0.1 μg/mL or less, measured in vitro; (3) block C3 activation with an IC₅₀ of about 0.1 μg/mL or less, measured in vitro; (4) block MAC activation with an IC₅₀ of about 0.1 μg/mL or less, measured in vitro; (5) do not affect the classical or alternative pathway of completement activation; or (6) any combination of (1)-(5).

In some embodiments, the antibodies or antigen-binding fragments provided herein specifically bind the protease domain of human MASP-2, wherein the antibodies or antigen-binding fragments: (1) bind human MASP-2 with a K_{D} that is 1 nM or less, measured by SPR; (2) block C4 activation with an IC₅₀ of 0.1 μg/mL or less, measured in vitro; (3) block C3 activation with an IC₅₀ of 0.1 μg/mL or less, measured in vitro; (4) block MAC activation with an IC₅₀ of 0.1 μg/mL or less, measured in vitro; (5) do not affect the classical or alternative pathway of completement activation; or (6) any combination of (1)-(5).

In some embodiments, the antibodies or antigen-binding fragments provided herein block C4 activation with an IC₅₀ of 0.001-0.01 μg/mL, measured in vitro. In some embodiments, the antibodies or antigen-binding fragments provided herein block C4 activation in the presence of 5-50% human serum with an IC₅₀ of 0.1 μg/mL or less, measured in vitro.

In some embodiments, the antibodies or antigen-binding fragments provided herein bind human MASP-2 with a K_{D} that ranges from 0.01 nM to 1 nM as measured by SPR. In some embodiments, the antibodies or antigen-binding fragments provided herein bind human MASP-2 with a K_{D} that ranges from 0.05 nM to 0.5 nM as measured by SPR.

Also provided herein are polynucleotides encoding the antibodies or antigen-binding fragments described herein. Also provided herein are vectors comprising the polynucleotides described herein. Also provided herein are host cells comprising the polynucleotides described herein or the vectors described herein.

Also provided herein are pharmaceutical compositions comprising a therapeutically effective amount of the antibodies or antigen-binding fragments described herein, and a pharmaceutically acceptable carrier.

Also provided herein are methods of inhibiting MASP-2-dependent complement activation in a subject in need thereof, comprising administering to the subject an effective amount of the antibodies or antigen-binding fragments described herein. In some embodiments, the subject has a disease or disorder associated with MASP-2-dependent complement activation.

In some embodiments, provided herein are methods of treating a disease or disorder associated with MASP-2-dependent complement activation in a subject in need thereof, comprising administering to the subject a therapeutically effective amount of the antibodies or antigen-binding fragments described herein. In some embodiments, the methods provided herein further comprise administering an additional therapy to the subject.

In some embodiments of the methods provided herein, the subject is a human.

Provided herein are also uses of the antibodies or antigen-binding fragments described herein in treating a disease or disorder associated with MASP-2-dependent complement activation.

Provided herein are also uses of the antibodies or antigen-binding fragments described herein for the preparation of a medicament for treating a disease or disorder associated with MASP-2-dependent complement activation.

In some embodiment of the methods or uses described herein, the disease or disorder is a renal disease or disorder, a vascular disease or disorder, a skin disease or disorder, an ophthalmologic disease or disorder, a nervous system disease or disorder, a blood disease or disorder, a musculoskeletal disease or disorder, a urogenital disease or disorder, a metabolic disease or disorder, an endocrine disease or disorder, a gastrointestinal disease or disorder, or a pulmonary disease or disorder.

In some embodiments, the disease or disorder is IgA nephropathy (IgAN), thrombotic microangiopathy (TMA), lupus nephritis (LN), membranous nephropathy (MN), or C3 glomerulopathy (C3G). In some embodiments, the disease or disorder is IgAN. In some embodiments, the disease or disorder is TMA.

In some embodiments, the disease or disorder is atypical hemolytic uremic syndrome (aHUS), TMA associated with hematopoietic stem cell transplantation (HSCT-TMA), thrombotic thrombocytopenia purpura (TTP), TMA secondary to cancer, TMA secondary to chemotherapy, or TMA secondary to transplantation. In some embodiments, the disease or disorder is HSCT-TMA. In some embodiments, the disease or disorder is TTP.

### 4. BRIEF DESCRIPTION OF DRAWINGS

FIG. 1 provides representative results from in vitro C4 activation assay showing the C4 blocking activities of various MASP-2 antibody clones. Clone 3E10 showed the strongest activity.

FIG. 2 provides representative results from in vitro C4 activation assay comparing the C4 blocking activity of 3E10 with that of the benchmark antibody narsoplimab (“MASP-2-BM”).

FIG. 3 provides biolayer interferometry (BLI) results measuring the binding affinity of 3E10 to human MASP-2.

FIG. 4 provides representative results from in vitro C3 activation assay comparing the C3 inhibiting activity of 3E10 with that of MASP-2-BM.

FIG. 5 provides representative results from in vitro MAC activation assay comparing the MAC inhibiting activity of 3E10 with that of MASP-2-BM.

FIG. 6 provides representative results from in vitro C4 activation assay showing that 3E10 blocked C4 activation in different concentrations (5%, 25%, and 50%) of human serum.

FIG. 7 provides representative results from in vitro C4 activation assay showing the C4 blocking activities of various humanized 3E10.

FIG. 8 provides representative results from surface plasmon resonance (SPR) measuring the binding affinity of a humanized 3E10 to human MASP-2.

FIG. 9 provides representative results from in vitro C4, C3, and MAC activation assays showing that humanized 3E10 inhibited the activation of the C4, C3 and MAC in serum from healthy donors.

FIG. 10 provides representative results from in vitro MAC activation assay showing that humanized 3E10 influenced neither the classical pathway nor alternative pathway-dependent activation.

FIG. 11 provides representative results from binding competition assay showing that 3E10 and MASP-2-BM did not compete for binding for human MASP-2.

FIG. 12 provides a diagram for truncated human MASP-2 proteins used in epitope mapping.

FIG. 13 provides representative results from binding assay using various truncated human MASP-2 proteins for epitope mapping of humanized 3E10.

### 5. DETAILED DESCRIPTION

The present disclosure provides novel antibodies, including antigen-binding fragments that specifically bind MASP-2 (e.g., human MASP-2). Pharmaceutical compositions comprising a therapeutically effective amount of such antibodies or antigen-binding fragments are also disclosed herein. Also disclosed herein are uses of such pharmaceutical compositions for treating a variety of disorders associated with complement activation (e.g., IgA nephropathy).

Before the present disclosure is further described, it is to be understood that the disclosure is not limited to the particular embodiments set forth herein, and it is also to be understood that the terminology used herein is for the purpose of describing particular embodiments, and is not intended to be limiting.

### 5.1 Definitions

Unless otherwise defined herein, scientific and technical terms used in the present disclosures shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The term “a” or “an” entity refers to one or more of that entity; for example, “an antibody,” is understood to represent one or more antibodies.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “antibody,” and its grammatical equivalents as used herein refer to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, single-domain antibodies (sdAbs; e.g., camelid antibodies, alpaca antibodies), single-chain Fv (scFv) antibodies, heavy chain antibodies (HCAbs), light chain antibodies (LCAbs), multispecific antibodies, bispecific antibodies, monospecific antibodies, monovalent antibodies, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, camel antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. Unless expressly indicated otherwise, the term “antibody” as used herein include “antigen-binding fragment” of intact antibodies. The term “antigen-binding fragment” as used herein refers to a portion or fragment of an intact antibody that is the antigenic determining variable region of an intact antibody. Examples of antigen-binding fragments include, but are not limited to, Fab, Fab′, F(ab′)2, Fv, linear antibodies, single chain antibody molecules (e.g., scFv), heavy chain antibodies (HCAbs), light chain antibodies (LCAbs), disulfide-linked scFv (dsscFv), diabodies, tribodies, tetrabodies, minibodies, dual variable domain antibodies (DVD), single variable domain antibodies (sdAbs; e.g., camelid antibodies, alpaca antibodies), and single variable domain of heavy chain antibodies (VHH), and bispecific or multispecific antibodies formed from antibody fragments. A “bispecific” antibody is an artificial hybrid antibody having two different antigen binding sites, which recognize and specifically bind two different targets. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulin. In some instances, the Fv framework region residues of a human immunoglobulin are replaced with the corresponding residues in an antibody from a non-human species. In some instances, residues of the CDRs are replaced by residues from the CDRs of a non-human species (e.g., mouse, rat, hamster, camel) that have the desired specificity, affinity, and/or binding capability. The humanized antibody can be further modified by the substitution of additional residues either in the Fv framework region and/or within the replaced non-human residues to refine and optimize antibody specificity, affinity, and/or binding capability. The term “human antibody” as used herein refers to an antibody produced by a human or an antibody having an amino acid sequence corresponding to an antibody produced by a human made using any of the techniques known in the art.

The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while μ and ε contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

The term “variable domain” or “variable region” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable domains differ extensively in sequence between different antibodies. The variability in sequence is concentrated in the CDRs while the less variable portions in the variable domain are referred to as framework regions (FR). The CDRs of the light and heavy chains are primarily responsible for the interaction of the antibody with antigen. Numbering of amino acid positions used herein is according to the EU Index, as in Kabat et al. (1991) Sequences of proteins of immunological interest. (U.S. Department of Health and Human Services, Washington, D.C.) 5thed. A variable region can be a human variable region.

A CDR refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences. CDR regions are well known to those skilled in the art and have been defined by a variety of methods/systems. These systems and/or definitions have been developed and refined over years and include Kabat, Chothia, IMGT, AbM, and Contact. For example, Kabat defines the regions of most hypervariability within the antibody variable (V) domains (Kabat et al, J. Biol. Chem. 252:6609-6616 (1977); Kabat, Adv. Prot. Chem. 32:1-75 (1978)). The Chothia definition is based on the location of the structural loop regions, which defines CDR region sequences as those residues that are not part of the conserved β-sheet framework, and thus are able to adapt different conformations (Chothia and Lesk, J. Mol. Biol. 196:901-917 (1987)). Both terminologies are well recognized in the art. Additionally, the IMGT system is based on sequence variability and location within the structure of the variable regions. The AbM definition is a compromise between Kabat and Chothia. The Contact definition is based on analyses of the available antibody crystal structures. Software programs (e.g., abYsis) are available and known to those of skill in the art for analysis of antibody sequence and determination of CDRs. The positions of CDRs within a canonical antibody variable domain have been determined by comparison of numerous structures (Al-Lazikani et al, J. Mol. Biol. 273:927-948 (1997); Morea et al, Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

For example, CDRs defined according to either the Kabat (hypervariable) or Chothia (structural) designations, are set forth in the table below.

| | Kabat¹ | Chothia² | Loop Location |
|---|---|---|---|
| VHCDRl | 31-35 | 26-32 | linking B and C strands |
| VHCDR2 | 50-65 | 53-55 | linking C′ and C″ strands |
| VHCDR3 | 95-102 | 96-101 | linking F and G strands |
| VLCDRl | 24-34 | 26-32 | linking B and C strands |
| VLCDR2 | 50-56 | 50-52 | linking C′ and C″ strands |
| VLCDR3 | 89-97 | 91-96 | linking F and G strands |

¹Residue numbering follows the nomenclature of Kabat et al., supra
²Residue numbering follows the nomenclature of Chothia et al., supra

One or more CDRs also can be incorporated into a molecule either covalently or noncovalently to make it an immunoadhesin. An immunoadhesin can incorporate the CDR(s) as part of a larger polypeptide chain, can covalently link the CDR(s) to another polypeptide chain, or can incorporate the CDR(s) noncovalently. The CDRs permit the immunoadhesin to bind to a particular antigen of interest. The CDR regions can be analyzed by, for example, abysis website (http://abysis.org/).

The terms “epitope” and “antigenic determinant” are used interchangeably herein an refer to the site on the surface of a target molecule to which an antibody or antigen-binding fragment binds, such as a localized region on the surface of an antigen. The target molecule can comprise, a protein, a peptide, a nucleic acid, a carbohydrate, or a lipid. An epitope having immunogenic activity is a portion of a target molecule that elicits an immune response in an animal. An epitope of a target molecule having antigenic activity is a portion of the target molecule to which an antibody binds, as determined by any method well known in the art, including, for example, by an immunoassay. Antigenic epitopes need not necessarily be immunogenic. Epitopes often consist of chemically active surface groupings of molecules such as amino acids or sugar side chains and have specific three dimensional structural characteristics as well as specific charge characteristics. The term, “epitope” includes linear epitopes and conformational epitopes. A region of a target molecule (e.g., a polypeptide) contributing to an epitope can be contiguous amino acids of the polypeptide or the epitope can come together from two or more non-contiguous regions of the target molecule. The epitope may or may not be a three-dimensional surface feature of the target molecule. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

The term “specifically binds,” as used herein, means that a polypeptide or molecule interacts more frequently, more rapidly, with greater duration, with greater affinity, or with some combination of the above to the epitope, protein, or target molecule than with alternative substances, including related and unrelated proteins. A binding moiety (e.g., antibody) that specifically binds a target molecule (e.g., antigen) can be identified, for example, by immunoassays, ELISAs, Bio-Layer Interferometry (“BLI”), SPR (e.g., Biacore), or other techniques known to those of skill in the art. Typically, a specific reaction will be at least twice background signal or noise and can be more than 10 times background. See, e.g., Paul, ed., 1989, Fundamental Immunology Second Edition, Raven Press, New York at pages 332-336 for a discussion regarding antibody specificity. A binding moiety that specifically binds a target molecule can bind the target molecule at a higher affinity than its affinity for a different molecule. In some embodiments, a binding moiety that specifically binds a target molecule can bind the target molecule with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different molecule. In some embodiments, a binding moiety that specifically binds a particular target molecule binds a different molecule at such a low affinity that binding cannot be detected using an assay described herein or otherwise known in the art. In some embodiments, “specifically binds” means, for instance, that a binding moiety binds a molecule target with a K_{D} of about 0.1 mM or less. In some embodiments, “specifically binds” means that a polypeptide or molecule binds a target with a K_{D} of at about 10 μM or less or about 1 μM or less. In some embodiments, “specifically binds” means that a polypeptide or molecule binds a target with a K_{D} of at about 0.1 μM or less, about 0.01 μM or less, or about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a binding moiety (e.g., antibody) that specifically binds a first target may or may not specifically bind a second target. As such, “specific binding” does not necessarily require (although it can include) exclusive binding, i.e., binding to a single target. Thus, a binding moiety (e.g., antibody) can, in some embodiments, specifically bind more than one target. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities.

The term “binding affinity” as used herein generally refers to the strength of the sum total of noncovalent interactions between a binding moiety and a target molecule (e.g., antigen). The binding of a binding moiety and a target molecule is a reversible process, and the affinity of the binding is typically reported as an equilibrium dissociation constant (K_{D}). K_{D} is the ratio of a dissociation rate (k_{off} or k_{d}) to the association rate (kₒₙ or k_{d}). The lower the K_{D} of a binding pair, the higher the affinity. A variety of methods of measuring binding affinity are known in the art, any of which can be used for purposes of the present disclosure. Specific illustrative embodiments include the following. In some embodiments, the “K_{D}” or “K_{D} value” can be measured by assays known in the art, for example by a binding assay. The K_{D} may be measured in a radiolabeled antigen binding assay (RIA) (Chen, et al., (1999) J. Mol Biol 293:865-881). The K_{D} or K_{D} value can also be measured by using biolayer interferometry (BLI) using, for example, the Gator system (Probe Life), or the Octet-96 system (Sartorius AG). The K_{D} or K_{D} value can also be measured by using surface plasmon resonance assays (SPR) by Biacore, using, for example, a BIAcore™-2000 or a BIAcore™-3000 BIAcore, Inc., Piscataway, NJ).

The term “variant” as used herein in relation to a protein or a polypeptide with particular sequence features (the “reference protein” or “reference polypeptide”) refers to a different protein or polypeptide having one or more (such as, for example, about 1 to about 25, about 1 to about 20, about 1 to about 15, about 1 to about 10, or about 1 to about 5) amino acid substitutions, deletions, and/or additions as compared to the reference protein or reference polypeptide. The changes to an amino acid sequence can be amino acid substitutions. The changes to an amino acid sequence can be conservative amino acid substitutions. A functional fragment or a functional variant of a protein or polypeptide maintains the basic structural and functional properties of the reference protein or polypeptide.

The terms “polypeptide,” “peptide,” “protein,” and their grammatical equivalents as used interchangeably herein refer to polymers of amino acids of any length, which can be linear or branched. It can include unnatural or modified amino acids or be interrupted by non-amino acids. A polypeptide, peptide, or protein can also be modified with, for example, disulfide bond formation, glycosylation, lipidation, acetylation, phosphorylation, or any other manipulation or modification.

The terms “polynucleotide,” “nucleic acid,” and their grammatical equivalents as used interchangeably herein mean polymers of nucleotides of any length and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical,” percent “identity,” and their grammatical equivalents as used herein in the context of two or more polynucleotides or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two polynucleotides or polypeptides provided herein are substantially identical, meaning they have at least 70%, at least 75%, at least 80%, at least 85%, at least 90%, and in some embodiments at least 95%, at least 96%, at least 97%, at least 98%, or at least 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

The term “vector,” and its grammatical equivalents as used herein refer to a vehicle that is used to carry genetic material (e.g., a polynucleotide sequence), which can be introduced into a host cell, where it can be replicated and/or expressed. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more polynucleotides are to be co-expressed, both polynucleotides can be inserted, for example, into a single expression vector or in separate expression vectors. For single vector expression, the encoding polynucleotides can be operationally linked to one common expression control sequence or linked to different expression control sequences, such as one inducible promoter and one constitutive promoter. The introduction of polynucleotides into a host cell can be confirmed using methods well known in the art. It is understood by those skilled in the art that the polynucleotides are expressed in a sufficient amount to produce a desired product (e.g., an anti-MASP-2 antibody or antigen-binding fragment as described herein), and it is further understood that expression levels can be optimized to obtain sufficient expression using methods well known in the art.

As used herein, the term “encode” and its grammatical equivalents refer to the inherent property of specific sequences of nucleotides in a polynucleotide or a nucleic acid, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein. Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

A polypeptide, peptide, protein, antibody, polynucleotide, vector, cell, or composition which is “isolated” is a polypeptide, peptide, protein, antibody, polynucleotide, vector, cell, or composition which is in a form not found in nature. Isolated polypeptides, peptides, proteins, antibodies, polynucleotides, vectors, cells, or compositions include those which have been purified to a degree that they are no longer in a form in which they are found in nature. In some embodiments, a polypeptide, peptide, protein, antibody, polynucleotide, vector, cell, or composition which is isolated is substantially pure.

The term “treat” and its grammatical equivalents as used herein in connection with a disease or a condition, or a subject having a disease or a condition refer to an action that suppresses, eliminates, reduces, and/or ameliorates a symptom, the severity of the symptom, and/or the frequency of the symptom associated with the disease or disorder being treated.

The term “administer” and its grammatical equivalents as used herein refer to the act of delivering, or causing to be delivered, a therapeutic or a pharmaceutical composition to the body of a subject by a method described herein or otherwise known in the art. The therapeutic can be a compound, a polypeptide, an antibody, a cell, or a population of cells. Administering a therapeutic or a pharmaceutical composition includes prescribing a therapeutic or a pharmaceutical composition to be delivered into the body of a subject. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

The terms “effective amount,” “therapeutically effective amount,” and their grammatical equivalents as used herein refer to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to the subject. The therapeutically effective amount can be ascertained by measuring relevant physiological effects. The exact amount required vary from subject to subject, depending on the age, weight, and general condition of the subject, the severity of the condition being treated, the judgment of the clinician, and the like. An appropriate “effective amount” in any individual case can be determined by one of ordinary skill in the art using routine experimentation.

The term “pharmaceutically acceptable carrier” or “pharmaceutically acceptable excipient” refers to a material that is suitable for drug administration to an individual along with an active agent without causing undesirable biological effects or interacting in a deleterious manner with any of the other components of the pharmaceutical composition.

The term “subject” as used herein refers to any animal (e.g., a mammal), including, but not limited to, humans, non-human primates, canines, felines, rodents, and the like, which is to be the recipient of a particular treatment. A subject can be a human. A subject can have a particular disease or condition.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Exemplary genes and polypeptides are described herein with reference to GenBank numbers, GI numbers and/or SEQ ID NOS. It is understood that one skilled in the art can readily identify homologous sequences by reference to sequence sources, including but not limited to GenBank (ncbi.nlm.nih.gov/genbank/) and EMBL (embl.org/).

### 5.2 Lectin Pathway and MASP-2

The complement system (CS) is an essential part of innate immunity. It is a network of more than 30 plasma and cell surface proteins that recognizes, labels, and eliminates microbial pathogens and dangerously altered (e.g., apoptotic) self-cells, triggers inflammation, and recruits immune cells. The CS can be activated through three pathways. The classical pathway is activated primarily by immune complexes, but it can also recognize microbial surfaces and apoptotic and necrotic cells; it contributes to the elimination of unnecessary synapses during ontogenesis; and it is important for the clearance of immune complexes and cell debris. The lectin pathway recognizes ancient surface-exposed molecular determinants on microbes via a diverse set of pattern recognition molecules (PRMs) and provides immediate defense against microbial pathogens, which does not depend on specific antibodies. The alternative pathway (AP) continuously challenges all surfaces by spontaneous low-level activation, but it activates productively only on those that lack protecting complement regulator molecules. Additionally, the AP provides an important amplification loop for complement activation.

Danger signal recognition triggers the activation of pathway-specific serine proteinase zymogens. The activated proteinases cleave downstream complement components that form surface-bound C3 convertases: C4b2a for the classical pathway and the lectin pathway and C3bBb for the alternative pathway. At this point, the three activation pathways converge to a common effector route leading to the labeling and lysis of the pathogens, recruitment of immune cells, and triggering of inflammation.

Normally, complement activation is tightly regulated. Lack of complement inhibition is responsible for the pathomechanism of many clinical conditions. In most complement-related diseases, the contribution of only one of the three pathways is dominant. Pathway-specific inhibitors, such as inhibitors of pathway-specific proteinases, can be ideal therapeutics that selectively block the derailed pathologic pathway while leaving the protecting functions of the other two pathways undisturbed.

Three mannan-binding lectin-associated serine proteases (MASP-1, MASP-2, and MASP-3) are presently known to be associated in human serum with the mannan-binding lectin (MBL), also known “mannose-binding protein.” The MBL-MASP-2 complex plays an important role in innate immunity by virtue of the binding of MBL to carbohydrate structures present on a wide variety of microorganisms. MASP-2 is activated upon binding of the recognition components to their respective pattern, and can also be activated by MASP-1, and subsequently cleaves the complement component C4 into C4a and C4b. After the binding of the cleavage product C4b to plasma C2, C4b-bound C2 becomes substrate of a second MASP-2-mediated cleavage step which converts C4b-bound C2 into the enzymatically active complex C4bC2a and a small C2b cleavage fragment. C4b2a is the C3-converting C3 convertase of the lectin pathway, converting the abundant plasma component C3 into C3a and C3b. C3b binds to any surface in close proximity via a thioester bond. If several C3b fragments bind in close proximity to the C3 convertase complex C4b2a, this convertase alters its specificity to convert C5 into C5b and C5a, forming the C5 convertase complex C4b2a (C3b) n. While this C5 convertase can initiate formation of the membrane attack complex or “MAC”, which refers to a complex of the terminal five complement components (C5b combined with C6, C7, C8 and C9) that inserts into and disrupts membranes (also referred to as C5b-9), this process is thought to be insufficiently effective to promote lysis on its own. Rather, the initial C3b opsonins produced by the lectin pathway form the nucleus for the formation of new alternative pathway C3 convertase and C5 convertase sites, which ultimately lead to abundant MAC formation and lysis. There is also a MASP-2-dependent C4-bypass activation route to activate C3 in the absence of C4, which plays an important role in the pathophysiology of ischemia-reperfusion injury.

As such, as used herein and understood in the art, “MASP-2-dependent complement activation” refers to the lectin pathway of complement activation that requires MASP-2. MASP-2-dependent complement activation occurs in the presence of Ca++, leading to the formation of the lectin pathway C3 convertase C4b2a and upon accumulation of the C3 cleavage product C3b subsequently to the C5 convertase C4b2a (C3b) n, which can cause opsonization and/or lysis.

The human MASP-2 gene is located on chromosome 1p36.3-2 (Stover et al., Cytogenet and Cell Genet 84:148-149 (1999) and encompasses twelve exons. The human MASP-2 cDNA is encoded by exons 2, 3, 4, 6, 7, 8, 9, 10, 11 and 12. The 20 kDa protein termed MBL-associated protein 19 (“MAp19”, also referred to as “sMAP”) arises from exons 2, 3, 4 and 5, which is a nonenzymatic protein containing the N-terminal CUB1-EGF region of MASP-2 with four additional residues (EQSL) derived from exon 5.

The MASP-2 polypeptide has 686 amino acid residues, which includes a leader peptide of 15 residues that is cleaved off after secretion, resulting in the mature form of human MASP-2 (671 amino acids; SEQ ID NO: 1). The MASP-2 polypeptide exhibits a molecular structure similar to MASP-1, MASP-3, and C1r and C1s, the proteases of the C1 complement system. A schematic diagram illustrating the domain structure of the human MASP-2 polypeptide is provided in FIG. 12 (labeled as “hMASP2-FL”). As shown, the serine proteases MASP-2 consists of six distinct domains: (1) an N-terminal C1r/C1s/sea urchin VEGF/bone morphogenic protein (or CUB1) domain (aa 1-122 of SEQ ID NO: 1); (2) an epidermal growth factor (EGF)-like domain (aa 123-166 of SEQ ID NO: 1); (3) a second CUB domain (CUB2) (aa 167-279 of SEQ ID NO: 1); (4 and 5) two complement control protein (CCP1 and CCP2) domains (CCP1 aa 280-345 and CCP2 aa 346-414 of SEQ ID NO: 1); and (6) a serine protease (SP) domain (aa 415-671 of SEQ ID NO: 1).

(SEQ ID NO: 1)
TPLGPKWPEPVFGRLASPGFPGEYANDQERRWTLTAPPGYRLRLYFTHFDLELSHLCEYDFVKL
SSGAKVLATLCGQESTDTERAPGKDTFYSLGSSLDITFRSDYSNEKPFTGFEAFYAAEDIDECQ
VAPGEAPTCDHHCHNHLGGFYCSCRAGYVLHRNKRTCSALCSGQVFTQRSGELSSPEYPRPYPK
LSSCTYSISLEEGFSVILDFVESEDVETHPETLCPYDFLKIQTDREEHGPFCGKTLPHRIETKS
NTVTITFVTDESGDHTGWKIHYTSTAQPCPYPMAPPNGHVSPVQAKYILKDSFSIFCETGYELL
QGHLPLKSFTAVCQKDGSWDRPMPACSIVDCGPPDDLPSGRVEYITGPGVTTYKAVIQYSCEET
FYTMKVNDGKYVCEADGFWTSSKGEKSLPVCEPVCGLSARTTGGRIYGGQKAKPGDFPWQVLIL
GGTTAAGALLYDNWVLTAAHAVYEQKHDASALDIRMGTLKRLSPHYTQAWSEAVFIHEGYTHDA
GFDNDIALIKLNNKVVINSNITPICLPRKEAESFMRTDDIGTASGWGLTQRGFLARNLMYVDIP
IVDHQKCTAAYEKPPYPRGSVTANMLCAGLESGGKDSCRGDSGGALVFLDSETERWFVGGIVSW
GSMNCGEAGQYGVYTKVINYIPWIENIISDF

The MASP-2 polypeptide has an alpha chain (heavy chain) containing the CUB1-EGF-CUB2-CCP1-CCP2 domains and a beta chain (light chain) containing the serine protease domain. The CUBl, EGF and CUB2 domains are required for dimerization and the CUBl, EGF, CUB2 and CCPl domains contain the binding site for MBP. Each MASP-2 dimer binds to two MBL subunits (Wallis et al., J. Biol Chem. 279:14065-14073 (2004)).

### 5.3 Anti-MASP-2 Antibodies and Antigen-Binding Fragments

Provided herein are antibodies or antigen-binding fragments thereof that specifically bind MASP-2 (e.g., human MASP-2). In some embodiments, provided herein are anti-MASP-2 antibodies. In some embodiments, the antibody is an IgA, IgD, IgE, IgG, or IgM antibody. In some embodiments, the antibody is an IgA antibody. In some embodiments, the antibody is an IgD antibody. In some embodiments, the antibody is an IgE antibody. In some embodiments, the antibody is an IgG antibody. In some embodiments, the antibody is an IgM antibody. In some embodiments, the antibodies provided herein can be an IgG1 antibody, an IgG2 antibody, an IgG3 antibody, or an IgG4 antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, provided herein are antigen-binding fragments of an anti-MASP-2 antibody. In some embodiments, antigen-binding fragments provided herein can be a single domain antibody (sdAb), a heavy chain antibody (HCAb), a Fab, a Fab′, a F(ab′)₂, a Fv, a single-chain variable fragment (scFv), or a (scFv)₂. In some embodiments, the antigen-binding fragment of an anti-MASP-2 antibody is a single domain antibody (sdAb). In some embodiments, the antigen-binding fragment of an anti-MASP-2 antibody is a heavy chain antibody (HCAb). In some embodiments, the antigen-binding fragment of an anti-MASP-2 antibody is a Fab. In some embodiments, the antigen-binding fragment of an anti-MASP-2 antibody is a Fab′. In some embodiments, the antigen-binding fragment of an anti-MASP-2 antibody is a F(ab′)₂. In some embodiments, the antigen-binding fragment of an anti-MASP-2 antibody is a Fv. In some embodiments, the antigen-binding fragment of an anti-MASP-2 antibody is a scFv. In some embodiments, the antigen-binding fragment of an anti-MASP-2 antibody is a disulfide-linked scFv [(scFv)₂]. In some embodiments, the antigen-binding fragment of an anti-MASP-2 antibody is a diabody (dAb).

In some embodiments, the anti-MASP-2 antibodies or antigen-binding fragments provided herein comprise recombinant antibodies or antigen-binding fragments. In some embodiments, the anti-MASP-2 antibodies or antigen-binding fragments provided herein comprise monoclonal antibodies or antigen-binding fragments. In some embodiments, the anti-MASP-2 antibodies or antigen-binding fragments provided herein comprise polyclonal antibodies or antigen-binding fragments. In some embodiments, the anti-MASP-2 antibodies or antigen-binding fragments provided herein comprise camelid (e.g., camels, dromedary and llamas) antibodies or antigen-binding fragments. In some embodiments, the anti-MASP-2 antibodies or antigen-binding fragments provided herein comprise chimeric antibodies or antigen-binding fragments. In some embodiments, the anti-MASP-2 antibodies or antigen-binding fragments provided herein comprise humanized antibodies or antigen-binding fragments. In some embodiments, the anti-MASP-2 antibodies or antigen-binding fragments provided herein comprise human antibodies or antigen-binding fragments. In some embodiments, provided herein are anti-MASP-2 human scFvs.

In some embodiments, the anti-MASP-2 antibodies or antigen-binding fragments provided herein are isolated. In some embodiments, the anti-MASP-2 antibodies or antigen-binding fragments provided herein are substantially pure.

In some embodiments, the anti-MASP-2 antibody or antigen-binding fragment provided herein comprises a multispecific antibody or antigen-binding fragment. In some embodiments, the anti-MASP-2 antibody or antigen-binding fragment provided herein comprises a bispecific antibody or antigen-binding fragment. In some embodiments, the bispecific antibody or antigen-binding fragment comprises an anti-MASP-2 antibody or antigen-binding fragment provided herein. In some embodiments, the bispecific antibody or antigen-binding fragment comprises an anti-MASP-2 scFv provided herein.

In some embodiments, the anti-MASP-2 antibody or antigen-binding fragment provided herein comprises a monovalent antigen-binding site. In some embodiments, an anti-MASP-2 antibody or antigen-binding fragment comprises a monospecific binding site. In some embodiments, an anti-MASP-2 antibody or antigen-binding fragment comprises a bivalent binding site.

In some embodiments, an anti-MASP-2 antibody or antigen-binding fragment is a monoclonal antibody or antigen-binding fragment. Monoclonal antibodies can be prepared by any method known to those of skill in the art. One exemplary approach is screening protein expression libraries, e.g., phage or ribosome display libraries. Phage display is described, for example, in Ladner et al., U.S. Pat. No. 5,223,409; Smith (1985) Science 228:1315-1317; and WO 92/18619. In some embodiments, recombinant monoclonal antibodies are isolated from phage display libraries expressing variable regions or CDRs of a desired species. Screening of phage libraries can be accomplished by various techniques known in the art.

In some embodiments, monoclonal antibodies are prepared using hybridoma methods known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is a human protein or a fragment thereof. In some embodiments, the immunizing antigen is a human protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed to a chosen antigen can be identified by a variety of methods including, but not limited to, immunoprecipitation, immunoblotting, and in vitro binding assays (e.g., flow cytometry, FACS, ELISA, SPR (e.g., Biacore), and radioimmunoassay). Once hybridoma cells that produce antibodies of the desired specificity, affinity, and/or activity are identified, the clones may be subcloned by limiting dilution or other techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies are made using recombinant DNA techniques as known to one skilled in the art. For example, the polynucleotides encoding an antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

In some embodiments, a monoclonal antibody is modified by using recombinant DNA technology to generate alternative antibodies. In some embodiments, the constant domains of the light chain and heavy chain of a mouse monoclonal antibody are replaced with the constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and/or affinity of a monoclonal antibody.

In some embodiments, an anti-MASP-2 antibody or antigen-binding fragment is a humanized antibody or antigen-binding fragment. Various methods for generating humanized antibodies are known in the art. Methods are known in the art for achieving high affinity binding with humanized antibodies. A non-limiting example of such a method is hypermutation of the variable region and selection of the cells expressing such high affinity antibodies (affinity maturation). In addition to the use of display libraries, the specified antigen (e.g., recombinant MASP-2 or an epitope thereof) can be used to immunize a non-human animal, e.g., a rodent. In certain embodiments, rodent antigen-binding fragments (e.g., mouse antigen-binding fragments) can be generated and isolated using methods known in the art and/or disclosed herein. In some embodiments, a mouse can be immunized with an antigen (e.g., recombinant MASP-2 or an epitope thereof).

In some embodiments, an anti-MASP-2 antibody or antigen-binding fragment is a human antibody or antigen-binding fragment. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology can be used to produce human antibodies and antibody fragments in vitro, from immunoglobulin variable region gene repertoires from unimmunized donors. Techniques for the generation and use of antibody phage libraries are well-known in the art. Once antibodies are identified, affinity maturation strategies known in the art, including but not limited to, chain shuffling and site-directed mutagenesis, can be employed to generate higher affinity human antibodies. In some embodiments, human antibodies are produced in transgenic mice that contain human immunoglobulin loci. Upon immunization these mice can produce the full repertoire of human antibodies in the absence of endogenous immunoglobulin production.

In some embodiments, provided herein is the anti-MASP-2 antibody clone 3E10. The sequence features are described below. The specific CDR sequences defined herein are generally based on either Kabat or IMGT definition. However, it is understood that a general reference to a heavy chain CDR or CDRs and/or a light chain CDR or CDRs of a specific antibody encompass all CDR definitions as known to those of skill in the art.

TABLE 1
Amino acid sequences of light chain variable region CDRs (VL CDRs)
of 3E10 and humanized 3E10

| | VL CDR1 | VL CDR2 | VL CDR3 |
|---|---|---|---|
| Kabat | KSSQNILYSSNQKNLLA | WASTRES | HQYFSSYT |
| | (SEQ ID NO: 7) | (SEQ ID NO: 9) | (SEQ ID NO: 11) |
| IMGT | QNILYSSNQKNL | WAS | HQYFSSYT |
| | (SEQ ID NO: 8) | (SEQ ID NO: 10) | (SEQ ID NO: 11) |

TABLE 2
Amino acid sequences of heavy chain variable region CDRs (VH CDRs) of
3E10 and humanized 3E10

| Sequence | VH CDR1 | VH CDR2 | VH CDR3 |
|---|---|---|---|
| Kabat | TYGVH | VIWKGGSTDYNAAFMS | TLELRRRDYYAMDY |
| | (SEQ ID NO: 12) | (SEQ ID NO: 16) | (SEQ ID NO: 18) |
| IMGT | GFSLTTYG | IWKGGST | VKTLELRRRDYYAMDY |
| | (SEQ ID NO: 13) | (SEQ ID NO: 17) | (SEQ ID NO: 19) |
| | GFTFSTYG | | AKTLELRRRDYYAMDY |
| | (SEQ ID NO: 14) | | (SEQ ID NO: 20) |
| | GFTLSTYG | | |
| | (SEQ ID NO: 15) | | |

Kabat numbering was followed for CDR grafting to generate humanized 3E10. As such, the Kabat CDRs remained the same after humanization while some IMGT CDRs showed some variation (1-3 amino acid substitutions).

In some embodiments, anti-MASP-2 antibodies or antigen-binding fragments provided herein comprise one, two, three, four, five, and/or six CDRs of any one of the antibodies described herein. In some embodiments, anti-MASP-2 antibodies or antigen-binding fragments provided herein comprise a light chain variable region (VL) comprising one, two, and/or three, light chain CDRs (VL CDRs) from Table 1. In some embodiments, anti-MASP-2 antibodies or antigen-binding fragments provided herein comprise a heavy chain variable region (VH) comprising one, two, and/or three heavy chain CDRs (VH CDRs) from Table 2. In some embodiments, anti-MASP-2 antibodies or antigen-binding fragments provided herein comprise one, two, and/or three VL CDRs from Table 1 and one, two, and/or three VH CDRs from Table 2.

In some embodiments, provided herein are antibodies or antigen-binding fragments thereof that specifically bind MASP-2, comprising a VL comprising (1) a VL CDR1 having the amino acid sequence of SEQ ID NO: 7 or 8; (2) a VL CDR2 having the amino acid sequence of SEQ ID NO: 9 or 10; or (3) a VL CDR3 having the amino acid sequence of SEQ ID NO: 11; or a variant thereof having up to about 3, about 5, about 8, about 10, about 12, or about 15 amino acid substitutions, additions, and/or deletions in the VL CDRs; and/or a VH comprising (1) a VH CDR1 having an amino acid sequence selected from the group consisting of SEQ ID NOs: 12-15; (2) a VH CDR2 having the amino acid sequence of SEQ ID NO: 16 or 17; or (3) a VH CDR3 having an amino acid sequence selected from the group consisting of SEQ ID NOs: 18-20; or a variant thereof having up to about 3, about 5, about 8, about 10, about 12, or about 15 amino acid substitutions, additions, and/or deletions in the VH CDRs.

In some embodiments, provided herein are antibodies or antigen-binding fragments thereof that specifically bind MASP-2, comprising a VL comprising (1) a VL CDR1 having the amino acid sequence of SEQ ID NO: 7 or 8; (2) a VL CDR2 having the amino acid sequence of SEQ ID NO: 9 or 10; or (3) a VL CDR3 having the amino acid sequence of SEQ ID NO: 11; or a variant thereof having up to about 3, about 5, about 8, about 10, about 12, or about 15 amino acid substitutions, additions, and/or deletions in the VL CDRs. In some embodiments, the variant has about 5 amino acid substitutions, additions, and/or deletions in the VL CDRs.

In some embodiments, provided herein are antibodies or antigen-binding fragments thereof that specifically bind MASP-2, comprising a VL comprising (1) a VL CDR1 having the amino acid sequence of SEQ ID NO: 7 or 8; (2) a VL CDR2 having the amino acid sequence of SEQ ID NO: 9 or 10; and (3) a VL CDR3 having the amino acid sequence of SEQ ID NO: 11; or a variant thereof having up to about 3, about 5, about 8, about 10, about 12, or about 15 amino acid substitutions, additions, and/or deletions in the VL CDRs. In some embodiments, the variant has up to about 5 amino acid substitutions, additions, and/or deletions in the VL CDRs.

In some embodiments, provided herein are antibodies or antigen-binding fragments thereof that specifically bind MASP-2 having a VL, wherein the VL comprises VL CDR1, CDR2 and CDR3 having the amino acid sequences of SEQ ID NOs: 7, 9 and 11, respectively, as defined by Kabat; or a variant thereof having up to about 3, about 5, about 8, about 10, about 12, or about 15 amino acid substitutions, additions, and/or deletions in the VL CDRs. In some embodiments, the variant has up about 5 amino acid substitutions, additions, and/or deletions in the VL CDRs. In some embodiments, provided herein are antibodies or antigen-binding fragments thereof that specifically bind MASP-2 having a VL, wherein the VL comprises VL CDR1, CDR2 and CDR3 having the amino acid sequences of SEQ ID NOs: 8, 10 and 11, respectively, as defined by IMGT; or a variant thereof having up to about 3, about 5, about 8, about 10, about 12, or about 15 amino acid substitutions, additions, and/or deletions in the VL CDRs. In some embodiments, the variant has up about 5 amino acid substitutions, additions, and/or deletions in the VL CDRs.

In some embodiments, provided herein are antibodies or antigen-binding fragments thereof that specifically bind MASP-2 comprising a VH comprising (1) a VH CDR1 having an amino acid sequence selected from the group consisting of SEQ ID NOs: 12-15; (2) a VH CDR2 having the amino acid sequence of SEQ ID NO: 16 or 17; or (3) a VH CDR3 having an amino acid sequence selected from the group consisting of SEQ ID NOs: 18-20; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind MASP-2 comprising a VH comprising (1) a VH CDR1 having an amino acid sequence selected from the group consisting of SEQ ID NOs: 12-15; (2) a VH CDR2 having the amino acid sequence of SEQ ID NO: 16 or 17; and (3) a VH CDR3 having an amino acid sequence selected from the group consisting of SEQ ID NOs: 18-20; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind MASP-2 having a VH, wherein the VH comprises VH CDR1, CDR2 and CDR3 having the amino acid sequences of SEQ ID NOs: 12, 16, and 18, respectively, as defined by Kabat; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind MASP-2 having a VH, wherein the VH comprises VH CDR1, CDR2 and CDR3 having the amino acid sequences of (1) SEQ ID NOs: 13, 17, and 19, respectively; (2) SEQ ID NOs: 14, 17, and 19, respectively; (3) SEQ ID NOs: 15, 17, and 19, respectively; (4) SEQ ID NOs: 13, 17, and 20, respectively; (5) SEQ ID NOs: 14, 17, and 20, respectively; or (6) SEQ ID NOs: 15, 17, and 20, respectively; as defined by IMGT; or a variant thereof having up to about 3, about 5, about 8, about 10, about 12, or about 15 amino acid substitutions, additions, and/or deletions in the VH CDRs. In some embodiments, the variant has up about 5 amino acid substitutions, additions, and/or deletions in the VH CDRs.

In some embodiments, provided herein are antibodies or antigen-binding fragments thereof that specifically bind MASP-2, comprising, as defined by Kabat, (a) a VL comprising VL CDR1, VL CDR2, VL CDR3 having the amino acid sequences of SEQ ID NOs: 7, 9, and 11, respectively; or a variant thereof having up to about 3, about 5, about 8, about 10, about 12, or about 15 amino acid substitutions, additions, and/or deletions in the VL CDRs; and/or (b) a VH comprising VH CDR1, VH CDR2, VH CDR3 having the amino acid sequences of SEQ ID NOs: 12, 16, and 18, respectively; or a variant thereof having up to having up to about 3, about 5, about 8, about 10, about 12, or about 15 amino acid substitutions, additions, and/or deletions in the VH CDRs.

In some embodiments, provided herein are antibodies or antigen-binding fragments thereof that specifically bind MASP-2 comprising VL CDR1, VL CDR2, VL CDR3, VH CDR1, VH CDR2 and VH CDR3, having the amino acid sequences of SEQ ID NOs: 7, 9, 11, 12, 16 and 18, respectively, as defined by Kabat, or a variant thereof having up to having up to about 3, about 5, about 8, about 10, about 12, or about 15 amino acid substitutions, additions, and/or deletions in the CDRs.

In some embodiments, provided herein are antibodies or antigen-binding fragments thereof that specifically bind MASP-2, comprising, as defined by IMGT (a) a VL comprising VL CDR1, VL CDR2, VL CDR3 having the amino acid sequences of SEQ ID NOs: 8, 10, and 11, respectively; or a variant thereof having up to about 3, about 5, about 8, about 10, about 12, or about 15 amino acid substitutions, additions, and/or deletions in the VL CDRs; and/or (b) a VH comprising VH CDR1, VH CDR2, VH CDR3 having the amino acid sequences of SEQ ID NOs: 13, 17, and 19, respectively; or a variant thereof having up to about 3, about 5, about 8, about 10, about 12, or about 15 amino acid substitutions, additions, and/or deletions in the VH CDRs.

In some embodiments, provided herein are antibodies or antigen-binding fragments thereof that specifically bind MASP-2 comprising VL CDR1 having the amino acid sequence of SEQ ID NO: 8; VL CDR2 having the amino acid sequence of SEQ ID NO: 10; VL CDR3 having the amino acid sequence SEQ ID NO: 11; VH CDR1 having the amino acid sequence of SEQ ID NO: 13, 14, or 15; VH CDR2 having the amino acid sequence of SEQ ID NO: 17; and VH CDR3 having the amino acid sequences of SEQ ID NO: 19 or 20; as defined by IMGT.

In some embodiments, provided herein are antibodies or antigen-binding fragments thereof that specifically bind MASP-2 comprising VL CDR1, VL CDR2, VL CDR3, VH CDR1, VH CDR2 and VH CDR3, having the amino acid sequences of SEQ ID NOs: 8, 10, 11, 13, 17, and 19, respectively, as defined by IMGT. In some embodiments, the VL CDR1, VL CDR2, VL CDR3, VH CDR1, VH CDR2 and VH CDR3 can have SEQ ID NOs: 8, 10, 11, 14, 17, and 19, respectively, as defined by IMGT. In some embodiments, the VL CDR1, VL CDR2, VL CDR3, VH CDR1, VH CDR2 and VH CDR3 can have SEQ ID NOs: 8, 10, 11, 15, 17, and 19, respectively, as defined by IMGT. In some embodiments, the VL CDR1, VL CDR2, VL CDR3, VH CDR1, VH CDR2 and VH CDR3 can have SEQ ID NOs: 8, 10, 11, 13, 17, and 20, respectively, as defined by IMGT. In some embodiments, the VL CDR1, VL CDR2, VL CDR3, VH CDR1, VH CDR2 and VH CDR3 can have SEQ ID NOs: 8, 10, 11, 14, 17, and 20, respectively, as defined by IMGT. In some embodiments, the VL CDR1, VL CDR2, VL CDR3, VH CDR1, VH CDR2 and VH CDR3 can have SEQ ID NOs: 8, 10, 11, 15, 17, and 19, respectively, as defined by IMGT.

TABLE 3
Amino acid sequences of light chain variable regions (VLs) and heavy chain
variable region (VHs) of anti-MASP-2 antibody 3E10 and humanized 3E10.

| Ab domain | Sequence |
|---|---|
| 3E10 VL | DIVMTQTTSSLAVSAGEKVTMSCKSSQNILYSSNQKNLLAWYQQKPGQSPKLLI |
| | YWASTRESGVPNRFTGSGSGTDFSLTINSVQPEDLAVYYCHQYFSSYTFGGGTK |
| | LEMK (SEQ ID NO: 21) |
| 3E10 VH | EVQLQQSGPGLVQSSQSLSITCTVSGFSLTTYGVHWVRLSPGKGLEWLGVIWKG |
| | GSTDYNAAFMSRLSITKDNSKSEVFFQMNSLQGDDSAIYYCVKTLELRRRDYYA |
| | MDYWGQGTSVTVSS (SEQ ID NO: 22) |
| 3E10-hu-VL | DIVMTQSPDSLAVSLGERATINCKSSQNILYSSNQKNLLAWYQQKPGQPPKLLIY |
| | WASTRESGVPDRFSGSGSGTDFTLTISSLQAEDVAVYYCHQYFSSYTFGQGTKLE |
| | IK (SEQ ID NO: 23) |
| 3E10-hu-VH-0 | QVQLVESGGGVVQPGRSLRLSCAASGFTFSTYGVHWVRQAPGKGLEWVAVIW |
| | KGGSTDYNAAFMSRFTISRDNSKNTIYIQMNSLRAEDTAVYYCAKTLELRRRDY |
| | YAMDYWGQGTLVTVSS (SEQ ID NO: 24) |
| 3E10-hu-VH-6 | QVQLVESGGGVVQPGRSLRLSCAASGFTLSTYGVHWVRQAPGKGLEWLGVIW |
| | KGGSTDYNAAFMSRLTISKDNSKNTVYIQMNSLRAEDTAVYYCAKTLELRRRD |
| | YYAMDYWGQGTLVTVSS (SEQ ID NO: 25) |
| 3E10-hu-VH-7-1 | QVQLVESGGGVVQPGRSLRLSCAASGFTLSTYGVHWVRQAPGKGLEWLGVIW |
| | KGGSTDYNAAFMSRLTISKDNSKSTVYIQMNSLRAEDTAVYYCAKTLELRRRD |
| | YYAMDYWGQGTLVTVSS (SEQ ID NO: 26) |
| 3E10-hu-VH-7-2 | QVQLVESGGGVVQPGRSLRLSCAASGFTLSTYGVHWVRQAPGKGLEWLGVIW |
| | KGGSTDYNAAFMSRLTISKDNSKNTVYIQMNSLRAEDTAVYYCVKTLELRRRD |
| | YYAMDYWGQGTLVTVSS (SEQ ID NO: 27) |
| 3E10-hu-VH-8 | QVQLVESGGGVVQPGRSLRLSCAASGFSLTTYGVHWVRQAPGKGLEWLGVIW |
| | KGGSTDYNAAFMSRLTISKDNSKNTVYIQMNSLRAEDTAVYYCAKTLELRRRD |
| | YYAMDYWGQGTLVTVSS (SEQ ID NO: 28) |
| 3E10-hu-VH-9 | QVQLVESGGGVVQPGRSLRLSCAASGFSLTTYGVHWVRQAPGKGLEWLGVIW |
| | KGGSTDYNAAFMSRLTISKDNSKSTVYIQMNSLRAEDTAVYYCAKTLELRRRD |
| | YYAMDYWGQGTLVTVSS (SEQ ID NO: 29) |
| 3E10-hu-VH-10 | QVQLVESGGGVVQPGRSLRLSCAASGFSLTTYGVHWVRQAPGKGLEWLGVIW |
| | KGGSTDYNAAFMSRLTISKDNSKSTVYIQMNSLRAEDTAVYYCVKTLELRRRD |
| | YYAMDYWGQGTLVTVSS (SEQ ID NO: 30) |
| 3E10-hu-VH-11 | QVQLVESGGGVVQPGRSLRISCAASGFSLTTYGVHWVRQAPGKGLEWLGVIWK |
| | GGSTDYNAAFMSRLTISKDNSKSTVYIQMNSLRAEDTAVYYCVKTLELRRRDY |
| | YAMDYWGQGTLVTVSS (SEQ ID NO: 31) |
| 3E10-hu-VH-12 | QVQLVESGGGVVQPGRSLRISCAVSGFSLTTYGVHWVRQAPGKGLEWLGVIWK |
| | GGSTDYNAAFMSRLTISKDNSKSTVYIQMNSLRAEDTAVYYCVKTLELRRRDY |
| | YAMDYWGQGTLVTVSS (SEQ ID NO: 32) |

In some embodiments, provided herein are antibodies or antigen-binding fragments thereof that specifically bind MASP-2 comprising a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 21. In some embodiments, provided herein are antibodies or antigen-binding fragments thereof that specifically bind MASP-2 comprising a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 22.

In some embodiments, provided herein are antibodies or antigen-binding fragments thereof that specifically bind MASP-2 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity sequence identity to the amino acid sequence of SEQ ID NO: 21; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity sequence identity to the amino acid sequence of SEQ ID NO: 22. In some embodiments, provided herein are antibodies or antigen-binding fragments thereof that specifically bind MASP-2 comprising a VL and a VH, wherein the VL and VH have the amino acid sequences of SEQ ID NOs: 21 and 22, respectively.

In some embodiments, provided herein are antibodies or antigen-binding fragments thereof that specifically bind MASP-2 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 21. In some embodiments, the anti-MASP-2 antibody or antigen-binding fragment thereof has a VL having at least 85% sequence identity to SEQ ID NO: 21. In some embodiments, the anti-MASP-2 antibody or antigen-binding fragment thereof has a VL having at least 90% sequence identity to SEQ ID NO: 21. In some embodiments, the anti-MASP-2 antibody or antigen-binding fragment thereof has a VL having at least 95% sequence identity to SEQ ID NO: 21. In some embodiments, the anti-MASP-2 antibody or antigen-binding fragment thereof has a VL having at least 98% sequence identity to SEQ ID NO: 21. In some embodiments, provided herein are antibodies or antigen-binding fragments thereof that specifically bind MASP-2 comprising a VL having the amino acid sequence of SEQ ID NO: 21.

In some embodiments, provided herein are antibodies or antigen-binding fragments thereof that specifically bind MASP-2 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 22. In some embodiments, the anti-MASP-2 antibody or antigen-binding fragment thereof has a VH having at least 85% sequence identity to SEQ ID NO: 22. In some embodiments, the anti-MASP-2 antibody or antigen-binding fragment thereof has a VH having at least 90% sequence identity to SEQ ID NO: 22. In some embodiments, the anti-MASP-2 antibody or antigen-binding fragment thereof has a VH having at least 95% sequence identity to SEQ ID NO: 22. In some embodiments, the anti-MASP-2 antibody or antigen-binding fragment thereof has a VH having at least 98% sequence identity to SEQ ID NO: 22. In some embodiments, provided herein are antibodies or antigen-binding fragments thereof that specifically bind MASP-2 comprising a VH having the amino acid sequence of SEQ ID NO: 22.

In some embodiments, an anti-MASP-2 antibody or antigen-binding fragment thereof comprises a humanized antibody or antigen-binding fragment. In some embodiments, an anti-MASP-2 antibody or antigen-binding fragment thereof comprises a VL CDR1, VL CDR2, VL CDR3, VH CDR1, VH CDR2, and/or VH CDR3 from an antibody or antigen-binding fragment described herein. In some embodiments, an anti-MASP-2 antibody or antigen-binding fragment thereof comprises a variant of an anti-MASP-2 antibody or antigen-binding fragment described herein. A variant of an anti-MASP-2 antibody or antigen-binding fragment can comprise one to 30 amino acid substitutions, additions, and/or deletions in the anti-MASP-2 antibody or antigen-binding fragment. A variant of an anti-MASP-2 antibody or antigen-binding fragment can comprise one to 25 amino acid substitutions, additions, and/or deletions in the anti-MASP-2 antibody or antigen-binding fragment. In some embodiments, a variant of an anti-MASP-2 antibody or antigen-binding fragment comprises one to 20 substitutions, additions, and/or deletions in the anti-MASP-2 antibody or antigen-binding fragment. In some embodiments, a variant of an anti-MASP-2 antibody or antigen-binding fragment comprises one to 15 substitutions, additions, and/or deletions in the anti-MASP-2 antibody or antigen-binding fragment. In some embodiments, a variant of an anti-MASP-2 antibody or antigen-binding fragment comprises one to 10 substitutions, additions, and/or deletions in the anti-MASP-2 antibody or antigen-binding fragment. In some embodiments, a variant of an anti-MASP-2 antibody or antigen-binding fragment comprises one to five conservative amino acid substitutions, additions, and/or deletions in the anti-MASP-2 antibody or antigen-binding fragment. In some embodiments, a variant of an anti-MASP-2 antibody or antigen-binding fragment comprises one to three amino acid substitutions, additions, and/or deletions in the anti-MASP-2 antibody or antigen-binding fragment. In some embodiments, the amino acid substitutions, additions, and/or deletions are conservative amino acid substitutions. In some embodiments, the conservative amino acid substitution(s) is in a CDR of the antibody or antigen-binding fragment. In some embodiments, the conservative amino acid substitution(s) is not in a CDR of the antibody or antigen-binding fragment. In some embodiments, the conservative amino acid substitution(s) is in a framework region of the antibody or antigen-binding fragment.

In some embodiments, provided herein are humanized antibodies or antigen-binding fragments thereof that specifically bind MASP-2 comprising: (a) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity sequence identity to the amino acid sequence of SEQ ID NO: 23; and (b) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity sequence identity to the amino acid sequence of SEQ ID NO: 24. In some embodiments, provided herein are antibodies or antigen-binding fragments thereof that specifically bind MASP-2 comprising a VL and a VH, wherein the VL and VH have the amino acid sequences of SEQ ID NOs: 23 and 24, respectively. In some embodiments, VL and VH have the amino acid sequences of SEQ ID NOs: 23 and 25, respectively. In some embodiments, VL and VH have the amino acid sequences of SEQ ID NOs: 23 and 26, respectively. In some embodiments, VL and VH have the amino acid sequences of SEQ ID NOs: 23 and 27, respectively. In some embodiments, VL and VH have the amino acid sequences of SEQ ID NOs: 23 and 28, respectively. In some embodiments, VL and VH have the amino acid sequences of SEQ ID NOs: 23 and 29, respectively. In some embodiments, VL and VH have the amino acid sequences of SEQ ID NOs: 23 and 30, respectively. In some embodiments, VL and VH have the amino acid sequences of SEQ ID NOs: 23 and 31, respectively. In some embodiments, VL and VH have the amino acid sequences of SEQ ID NOs: 23 and 32, respectively.

In some embodiments, provided herein are humanized antibodies or antigen-binding fragments thereof that specifically bind MASP-2 comprising a VL, wherein the VL has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 23. The humanized anti-MASP-2 antibody or antigen-binding fragment thereof can have a VL having at least 85% sequence identity to SEQ ID NO: 23. The humanized anti-MASP-2 antibody or antigen-binding fragment thereof can have a VL having at least 90% sequence identity to SEQ ID NO: 23. The humanized anti-MASP-2 antibody or antigen-binding fragment thereof can have a VL having at least 95% sequence identity to SEQ ID NO: 23. The humanized anti-MASP-2 antibody or antigen-binding fragment thereof can have a VL having at least 98% sequence identity to SEQ ID NO: 23. In some embodiments, provided herein are humanized antibodies or antigen-binding fragments thereof that specifically bind MASP-2 comprising a VL having the amino acid sequence of SEQ ID NO: 23.

In some embodiments, provided herein are humanized antibodies or antigen-binding fragments thereof that specifically bind MASP-2 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 24. The humanized anti-MASP-2 antibody or antigen-binding fragment thereof can have a VH having at least 85% sequence identity to SEQ ID NO: 24. The humanized anti-MASP-2 antibody or antigen-binding fragment thereof can have a VH having at least 90% sequence identity to SEQ ID NO: 24. The humanized anti-MASP-2 antibody or antigen-binding fragment thereof can have a VH having at least 95% sequence identity to SEQ ID NO: 24. The humanized anti-MASP-2 antibody or antigen-binding fragment thereof can have a VH having at least 98% sequence identity to SEQ ID NO: 24. In some embodiments, provided herein are humanized antibodies or antigen-binding fragments thereof that specifically bind MASP-2 comprising a VH having the amino acid sequence of SEQ ID NO: 24.

In some embodiments, provided herein are humanized antibodies or antigen-binding fragments thereof that specifically bind MASP-2 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 25. The humanized anti-MASP-2 antibody or antigen-binding fragment thereof can have a VH having at least 85% sequence identity to SEQ ID NO: 25. The humanized anti-MASP-2 antibody or antigen-binding fragment thereof can have a VH having at least 90% sequence identity to SEQ ID NO: 25. The humanized anti-MASP-2 antibody or antigen-binding fragment thereof can have a VH having at least 95% sequence identity to SEQ ID NO: 25. The humanized anti-MASP-2 antibody or antigen-binding fragment thereof can have a VH having at least 98% sequence identity to SEQ ID NO: 25. In some embodiments, provided herein are humanized antibodies or antigen-binding fragments thereof that specifically bind MASP-2 comprising a VH having the amino acid sequence of SEQ ID NO: 25.

In some embodiments, provided herein are humanized antibodies or antigen-binding fragments thereof that specifically bind MASP-2 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 26. The humanized anti-MASP-2 antibody or antigen-binding fragment thereof can have a VH having at least 85% sequence identity to SEQ ID NO: 26. The humanized anti-MASP-2 antibody or antigen-binding fragment thereof can have a VH having at least 90% sequence identity to SEQ ID NO: 26. The humanized anti-MASP-2 antibody or antigen-binding fragment thereof can have a VH having at least 95% sequence identity to SEQ ID NO: 26. The humanized anti-MASP-2 antibody or antigen-binding fragment thereof can have a VH having at least 98% sequence identity to SEQ ID NO: 26. In some embodiments, provided herein are humanized antibodies or antigen-binding fragments thereof that specifically bind MASP-2 comprising a VH having the amino acid sequence of SEQ ID NO: 26.

In some embodiments, provided herein are humanized antibodies or antigen-binding fragments thereof that specifically bind MASP-2 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 27. The humanized anti-MASP-2 antibody or antigen-binding fragment thereof can have a VH having at least 85% sequence identity to SEQ ID NO: 27. The humanized anti-MASP-2 antibody or antigen-binding fragment thereof can have a VH having at least 90% sequence identity to SEQ ID NO: 27. The humanized anti-MASP-2 antibody or antigen-binding fragment thereof can have a VH having at least 95% sequence identity to SEQ ID NO: 27. The humanized anti-MASP-2 antibody or antigen-binding fragment thereof can have a VH having at least 98% sequence identity to SEQ ID NO: 27. In some embodiments, provided herein are humanized antibodies or antigen-binding fragments thereof that specifically bind MASP-2 comprising a VH having the amino acid sequence of SEQ ID NO: 27.

In some embodiments, provided herein are humanized antibodies or antigen-binding fragments thereof that specifically bind MASP-2 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 28. The humanized anti-MASP-2 antibody or antigen-binding fragment thereof can have a VH having at least 85% sequence identity to SEQ ID NO: 28. The humanized anti-MASP-2 antibody or antigen-binding fragment thereof can have a VH having at least 90% sequence identity to SEQ ID NO: 28. The humanized anti-MASP-2 antibody or antigen-binding fragment thereof can have a VH having at least 95% sequence identity to SEQ ID NO: 28. The humanized anti-MASP-2 antibody or antigen-binding fragment thereof can have a VH having at least 98% sequence identity to SEQ ID NO: 28. In some embodiments, provided herein are humanized antibodies or antigen-binding fragments thereof that specifically bind MASP-2 comprising a VH having the amino acid sequence of SEQ ID NO: 28.

In some embodiments, provided herein are humanized antibodies or antigen-binding fragments thereof that specifically bind MASP-2 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 29. The humanized anti-MASP-2 antibody or antigen-binding fragment thereof can have a VH having at least 85% sequence identity to SEQ ID NO: 29. The humanized anti-MASP-2 antibody or antigen-binding fragment thereof can have a VH having at least 90% sequence identity to SEQ ID NO: 29. The humanized anti-MASP-2 antibody or antigen-binding fragment thereof can have a VH having at least 95% sequence identity to SEQ ID NO: 29. The humanized anti-MASP-2 antibody or antigen-binding fragment thereof can have a VH having at least 98% sequence identity to SEQ ID NO: 29. In some embodiments, provided herein are humanized antibodies or antigen-binding fragments thereof that specifically bind MASP-2 comprising a VH having the amino acid sequence of SEQ ID NO: 29.

In some embodiments, provided herein are humanized antibodies or antigen-binding fragments thereof that specifically bind MASP-2 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 30. The humanized anti-MASP-2 antibody or antigen-binding fragment thereof can have a VH having at least 85% sequence identity to SEQ ID NO: 30. The humanized anti-MASP-2 antibody or antigen-binding fragment thereof can have a VH having at least 90% sequence identity to SEQ ID NO: 30. The humanized anti-MASP-2 antibody or antigen-binding fragment thereof can have a VH having at least 95% sequence identity to SEQ ID NO: 30. The humanized anti-MASP-2 antibody or antigen-binding fragment thereof can have a VH having at least 98% sequence identity to SEQ ID NO: 30. In some embodiments, provided herein are humanized antibodies or antigen-binding fragments thereof that specifically bind MASP-2 comprising a VH having the amino acid sequence of SEQ ID NO: 30.

In some embodiments, provided herein are humanized antibodies or antigen-binding fragments thereof that specifically bind MASP-2 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 31. The humanized anti-MASP-2 antibody or antigen-binding fragment thereof can have a VH having at least 85% sequence identity to SEQ ID NO: 31. The humanized anti-MASP-2 antibody or antigen-binding fragment thereof can have a VH having at least 90% sequence identity to SEQ ID NO: 31. The humanized anti-MASP-2 antibody or antigen-binding fragment thereof can have a VH having at least 95% sequence identity to SEQ ID NO: 31. The humanized anti-MASP-2 antibody or antigen-binding fragment thereof can have a VH having at least 98% sequence identity to SEQ ID NO: 31. In some embodiments, provided herein are humanized antibodies or antigen-binding fragments thereof that specifically bind MASP-2 comprising a VH having the amino acid sequence of SEQ ID NO: 31.

In some embodiments, provided herein are humanized antibodies or antigen-binding fragments thereof that specifically bind MASP-2 comprising a VH, wherein the VH has at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 32. The humanized anti-MASP-2 antibody or antigen-binding fragment thereof can have a VH having at least 85% sequence identity to SEQ ID NO: 32. The humanized anti-MASP-2 antibody or antigen-binding fragment thereof can have a VH having at least 90% sequence identity to SEQ ID NO: 32. The humanized anti-MASP-2 antibody or antigen-binding fragment thereof can have a VH having at least 95% sequence identity to SEQ ID NO: 32. The humanized anti-MASP-2 antibody or antigen-binding fragment thereof can have a VH having at least 98% sequence identity to SEQ ID NO: 32. In some embodiments, provided herein are humanized antibodies or antigen-binding fragments thereof that specifically bind MASP-2 comprising a VH having the amino acid sequence of SEQ ID NO: 32.

The anti-MASP-2 antibodies or antigen-binding fragments thereof can comprise a combination of any VL disclosed herein and any VH disclosed herein. In some embodiments, the VL and VH are connected by a linker. The linker can be a flexible linker or a rigid linker. In some embodiments, the linker has the amino acid sequence of (GGGGS)n, n=1, 2, 3, 4, or 5 (SEQ ID NO: 77). In some embodiments, the linker has the amino acid sequence of (EAAAK)n, n=1, 2, 3, 4, or 5 (SEQ ID NO: 78). In some embodiments, the linker has the amino acid sequence of (PA)nP, n=1, 2, 3, 4, or 5 (SEQ ID NO: 79).

In some embodiments, provided herein are antibodies or antigen-binding fragments thereof that specifically bind MASP-2 comprising (a) a VL comprising VL CDRs 1, 2, and 3 from a VL having the amino acid sequence of SEQ ID NO: 21 or 23; and/or (b) a VH comprising VH CDRs 1, 2, and 3 from a VH having an amino acid sequence selected from the group consisting of SEQ ID NOs: 22 and 24-32.

In some embodiments, provided herein are antibodies or antigen-binding fragments thereof that specifically bind MASP-2 comprising a VL, wherein the VL comprises VL CDRs 1, 2, and 3 from a VL having the amino acid sequence of SEQ ID NO: 21. In some embodiments, provided herein are antibodies or antigen-binding fragments thereof that specifically bind MASP-2 comprising a VL, wherein the VL comprises VL CDRs 1, 2, and 3 from a VL having the amino acid sequence of SEQ ID NO: 23.

In some embodiments, provided herein are humanized antibodies or antigen-binding fragments thereof that specifically bind MASP-2 comprising a VH, wherein the VH comprises VH CDRs 1, 2, and 3 from a VH having the amino acid sequence of SEQ ID NO: 22. In some embodiments, provided herein are antibodies or antigen-binding fragments thereof that specifically bind MASP-2 comprising a VH, wherein the VH comprises VH CDRs 1, 2, and 3 from a VH having the amino acid sequence of SEQ ID NO: 24. In some embodiments, provided herein are antibodies or antigen-binding fragments thereof that specifically bind MASP-2 comprising a VH, wherein the VH comprises VH CDRs 1, 2, and 3 from a VH having the amino acid sequence of SEQ ID NO: 25. In some embodiments, provided herein are antibodies or antigen-binding fragments thereof that specifically bind MASP-2 comprising a VH, wherein the VH comprises VH CDRs 1, 2, and 3 from a VH having the amino acid sequence of SEQ ID NO: 26. In some embodiments, provided herein are antibodies or antigen-binding fragments thereof that specifically bind MASP-2 comprising a VH, wherein the VH comprises VH CDRs 1, 2, and 3 from a VH having the amino acid sequence of SEQ ID NO: 27. In some embodiments, provided herein are antibodies or antigen-binding fragments thereof that specifically bind MASP-2 comprising a VH, wherein the VH comprises VH CDRs 1, 2, and 3 from a VH having the amino acid sequence of SEQ ID NO: 28. In some embodiments, provided herein are antibodies or antigen-binding fragments thereof that specifically bind MASP-2 comprising a VH, wherein the VH comprises VH CDRs 1, 2, and 3 from a VH having the amino acid sequence of SEQ ID NO: 29. In some embodiments, provided herein are antibodies or antigen-binding fragments thereof that specifically bind MASP-2 comprising a VH, wherein the VH comprises VH CDRs 1, 2, and 3 from a VH having the amino acid sequence of SEQ ID NO: 30. In some embodiments, provided herein are antibodies or antigen-binding fragments thereof that specifically bind MASP-2 comprising a VH, wherein the VH comprises VH CDRs 1, 2, and 3 from a VH having the amino acid sequence of SEQ ID NO: 31. In some embodiments, provided herein are antibodies or antigen-binding fragments thereof that specifically bind MASP-2 comprising a VH, wherein the VH comprises VH CDRs 1, 2, and 3 from a VH having the amino acid sequence of SEQ ID NO: 32.

In some embodiments, provided herein are antibodies or antigen-binding fragments thereof that specifically bind MASP-2 comprising a VL and a VH, wherein the VL comprises VL CDR1, CDR2, and CDR3 from a VL having the amino acid sequence of SEQ ID NO: 21, and the VH comprises VH CDR1, CDR2, and CDR3 from a VH having the amino acid sequence of SEQ ID NO: 22. In some embodiments, provided herein are antibodies or antigen-binding fragments thereof that specifically bind MASP-2 comprising a VL and a VH, wherein the VL comprises VL CDR1, CDR2, and CDR3 from a VL having the amino acid sequence of SEQ ID NO: 23, and the VH comprises VH CDR1, CDR2, and CDR3 from a VH having the amino acid sequence of SEQ ID NO: 24. In some embodiments, the VL comprises VL CDR1, CDR2, and CDR3 from a VL having the amino acid sequence of SEQ ID NO: 23, and the VH comprises VH CDR1, CDR2, and CDR3 from a VH having the amino acid sequence of SEQ ID NO: 25. In some embodiments, the VL comprises VL CDR1, CDR2, and CDR3 from a VL having the amino acid sequence of SEQ ID NO: 23, and the VH comprises VH CDR1, CDR2, and CDR3 from a VH having the amino acid sequence of SEQ ID NO: 26. In some embodiments, the VL comprises VL CDR1, CDR2, and CDR3 from a VL having the amino acid sequence of SEQ ID NO: 23, and the VH comprises VH CDR1, CDR2, and CDR3 from a VH having the amino acid sequence of SEQ ID NO: 27. In some embodiments, the VL comprises VL CDR1, CDR2, and CDR3 from a VL having the amino acid sequence of SEQ ID NO: 23, and the VH comprises VH CDR1, CDR2, and CDR3 from a VH having the amino acid sequence of SEQ ID NO: 28. In some embodiments, the VL comprises VL CDR1, CDR2, and CDR3 from a VL having the amino acid sequence of SEQ ID NO: 23, and the VH comprises VH CDR1, CDR2, and CDR3 from a VH having the amino acid sequence of SEQ ID NO: 29. In some embodiments, the VL comprises VL CDR1, CDR2, and CDR3 from a VL having the amino acid sequence of SEQ ID NO: 23, and the VH comprises VH CDR1, CDR2, and CDR3 from a VH having the amino acid sequence of SEQ ID NO: 30. In some embodiments, the VL comprises VL CDR1, CDR2, and CDR3 from a VL having the amino acid sequence of SEQ ID NO: 23, and the VH comprises VH CDR1, CDR2, and CDR3 from a VH having the amino acid sequence of SEQ ID NO: 31. In some embodiments, the VL comprises VL CDR1, CDR2, and CDR3 from a VL having the amino acid sequence of SEQ ID NO: 23, and the VH comprises VH CDR1, CDR2, and CDR3 from a VH having the amino acid sequence of SEQ ID NO: 32.

In some embodiments, the anti-MASP-2 antibody or antigen-binding fragment thereof provided herein is the antibody designated as 3E10. In some embodiments, the anti-MASP-2 antibody or antigen-binding fragment thereof provided herein has a VL from 3E10 (SEQ ID NO: 21). In some embodiments, the anti-MASP-2 antibody or antigen-binding fragment thereof provided herein has a VH from 3E10 (SEQ ID NO: 22). The anti-MASP-2 antibody or antigen-binding fragment thereof provided herein can have both a VL and a VH from 3E10. In some embodiments, the anti-MASP-2 antibody or antigen-binding fragment thereof provided herein has a VL that comprises VL CDRs 1, 2, and 3 from the VL from 3E10 (SEQ ID NO: 21). In some embodiments, the anti-MASP-2 antibody or antigen-binding fragment thereof provided herein has a VH that comprises VH CDRs 1, 2, and 3 from the VH from 3E10 (SEQ ID NO: 22). The anti-MASP-2 antibody or antigen-binding fragment thereof provided herein can have a VL comprising VL CDRs 1, 2, and 3 and a VH comprising VH CDRs 1, 2, and 3 from the VL and VH of 3E10, respectively. In some embodiments, the anti-MASP-2 antibody or antigen-binding fragment thereof provided herein is a variant of 3E10. The 3E10 variant can have a VL that is a variant of the VL of 3E10 having up to about 5 amino acid substitutions, additions, and/or deletions in SEQ ID NO: 21. The 3E10 variant can have a VH that is a variant of the VH of 3E10 having up to about 5 amino acid substitutions, additions, and/or deletions in SEQ ID NO: 22. The amino acid substitutions, additions, and/or deletions can be in the VH CDRs or VL CDRs. In some embodiments, the amino acid substitutions, additions, and/or deletions are not in the CDRs. In some embodiments, the variant of 3E10 has up to about 5 conservative amino acid substitutions. In some embodiments, the variant of 3E10 has up to 3 conservative amino acid substitutions. In some embodiments, the anti-MASP-2 antibody or antigen-binding fragment thereof provided herein is a humanized antibody or antigen-binding fragment derived from 3E10. In some embodiments, the anti-MASP-2 antibody or antigen-binding fragment thereof provided herein is a human antibody or antigen-binding fragment derived from 3E10.

In some embodiments, provided herein are humanized 3E10. In some embodiments, the humanized anti-MASP-2 antibody or antigen-binding fragment thereof provided herein comprises a VL having the amino acid sequence of SEQ ID NO: 23. In some embodiments, the humanized anti-MASP-2 antibody or antigen-binding fragment thereof provided herein comprises a VH having an amino acid sequence selected from SEQ ID NOs: 24-32. In some embodiments, the humanized anti-MASP-2 antibody or antigen-binding fragment thereof provided herein comprises a VL having the amino acid sequence of SEQ ID NO: 23 and a VH having an amino acid sequence selected from SEQ ID NOs: 24-32. In some embodiments, the anti-MASP-2 antibody or antigen-binding fragment thereof provided herein is a variant of a humanized 3E10 provided herein. The variant can have a VL that is a variant of the VL of a humanized 3E10 having up to about 5 amino acid substitutions, additions, and/or deletions in the amino acid sequence of SEQ ID NO: 23. The variant can have a VH that is a variant of the VH of a humanized 3E10 having up to about 5 amino acid substitutions, additions, and/or deletions in an amino acid sequence selected from the group consisting of SEQ ID NOs: 24-32. In some embodiments, the variant of a humanized 3E10 has up to about 5 conservative amino acid substitutions.

In some embodiments, provided herein are also antibodies or antigen-binding fragments that compete with the antibody or antigen-binding fragment provided above for binding to MASP-2 (e.g., human MASP-2). Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments, e.g., BIACORE® surface plasmon resonance (SPR) analysis. In some embodiments, an anti-MASP-2 antibody or antigen-binding fragment competes with, and inhibits binding of another antibody or antigen-binding fragment to MASP-2 by at least 50%, 60%, 70%, 80%, 90% or 100%. Competition assays can be conducted as described, for example, in Ed Harlow and David Lane, Cold Spring Harb Protoc; 2006; doi: 10.H01/pdb.prot4277 or in Chapter 11 of “Using Antibodies” by Ed Harlow and David Lane, Cold Spring Harbor Laboratory Press, Cold Spring Harbor, NY, USA 1999.

In some embodiments, provided herein are antibodies or antigen-binding fragments that compete with an anti-MASP-2 antibody or antigen-binding fragment disclosed herein for binding to human MASP-2. In some embodiments, provided herein are antibodies or antigen-binding fragments that compete with 3E10 for binding to human MASP-2. In some embodiments, provided herein are antibodies or antigen-binding fragments that compete with a humanized 3E10 disclosed herein for binding to human MASP-2.

The present disclosure further contemplates additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, and/or solubility. Those skilled in the art will appreciate that amino acid changes may alter post-translational processes of an antibody, such as changing the number or position of glycosylation sites or altering membrane anchoring characteristics.

Variations can be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, e.g., conservative amino acid replacements. Insertions or deletions can be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/or relevant can be determined by systematically making insertions, deletions, or substitutions in the sequence and testing the resulting variant proteins for activity as compared to the parent protein.

It is known in the art that the constant region(s) of an antibody mediates several effector functions and these effector functions can vary depending on the isotype of the antibody. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In some embodiments, anti-MASP-2 antibody or antigen-binding fragment described herein comprise at least one constant region of a human IgA antibody. In some embodiments, anti-MASP-2 antibody or antigen-binding fragment described herein comprise at least one constant region of a human IgD antibody. In some embodiments, anti-MASP-2 antibody or antigen-binding fragment described herein comprise at least one constant region of a human IgE antibody. In some embodiments, anti-MASP-2 antibody or antigen-binding fragment described herein comprise at least one constant region of a human IgG antibody. In some embodiments, anti-MASP-2 antibody or antigen-binding fragment described herein comprise at least one constant region of a human IgM antibody. In some embodiments, anti-MASP-2 antibody or antigen-binding fragment described herein comprise at least one constant region of a human IgG1 antibody. In some embodiments, anti-MASP-2 antibody or antigen-binding fragment described herein comprise at least one constant region of a human IgG2 antibody. In some embodiments, anti-MASP-2 antibody or antigen-binding fragment described herein comprise at least one constant region of a human IgG3 antibody. In some embodiments, anti-MASP-2 antibody or antigen-binding fragment described herein comprise at least one constant region of a human IgG4 antibody.

In some embodiments, at least one or more of the constant regions has been modified or deleted in the anti-MASP-2 antibody or antigen-binding fragment described herein. In some embodiments, the antibodies comprise modifications to one or more of the three heavy chain constant regions (CH1, CH2 or CH3) and/or to the light chain constant region (CL). In some embodiments, the heavy chain constant region of the modified antibodies comprises at least one human constant region. In some embodiments, the heavy chain constant region of the modified antibodies comprises more than one human constant region. In some embodiments, modifications to the constant region comprise additions, deletions, or substitutions of one or more amino acids in one or more regions. In some embodiments, one or more regions are partially or entirely deleted from the constant regions of the modified antibodies. In some embodiments, the entire CH2 domain has been removed from an antibody (ΔCH2 constructs). In some embodiments, a deleted constant region is replaced by a short amino acid spacer that provides some of the molecular flexibility typically imparted by the absent constant region. In some embodiments, a modified antibody comprises a CH3 domain directly fused to the hinge region of the antibody. In some embodiments, a modified antibody comprises a peptide spacer inserted between the hinge region and modified CH2 and/or CH3 domains.

In some embodiments, an anti-MASP-2 antibody or antigen-binding fragment comprises a Fc region. In some embodiments, the Fc region is fused via a hinge. The hinge can be an IgG1 hinge, an IgG2 hinge, or an IgG3 hinge. The amino acid sequences of the Fc region of human IgG1, IgG2, IgG3, and IgG4 are known to those of ordinary skill in the art. In some cases, Fc regions with amino acid variations have been identified in native antibodies. In some embodiments, the modified antibodies (e.g., modified Fc region) provide for altered effector functions that, in turn, affect the biological profile of the antibody. For example, in some embodiments, the deletion or inactivation (through point mutations or other means) of a constant region reduces Fc receptor binding of the modified antibody as it circulates. In some embodiments, the constant region modifications reduce the immunogenicity of the antibody. In some embodiments, the constant region modifications increase the serum half-life of the antibody. In some embodiments, the constant region modifications reduce the serum half-life of the antibody. In some embodiments, the constant region modifications decrease or remove ADCC and/or complement dependent cytotoxicity (CDC) of the antibody. In some embodiments, specific amino acid substitutions in a human IgG1 Fc region with corresponding IgG2 or IgG4 residues reduce effector functions (e.g., ADCC and CDC) in the modified antibody. In some embodiments, an antibody does not have one or more effector functions (e.g., “effectorless” antibodies). In some embodiments, the antibody has no ADCC activity and/or no CDC activity. In some embodiments, the antibody does not bind an Fc receptor and/or complement factors. In some embodiments, the antibody has no effector function(s). In some embodiments, the constant region modifications increase or enhance ADCC and/or CDC of the antibody. In some embodiments, the constant region is modified to eliminate disulfide linkages or oligosaccharide moieties. In some embodiments, the constant region is modified to add/substitute one or more amino acids to provide one or more cytotoxin, oligosaccharide, or carbohydrate attachment sites. In some embodiments, an anti-MASP-2 antibody or antigen-binding fragment comprises a variant Fc region that is engineered with substitutions at specific amino acid positions as compared to a native Fc region. In some embodiments, an anti-MASP-2 antibody or antigen-binding fragment described herein comprises an IgG1 heavy chain constant region that comprises one or more amino acid substitutions selected from the group consisting of K214, L234, L235, G237, D356, and L358, numbered according to the EU Index. The K214 substitution can be, e.g., K214R. The L234 substitution can be, e.g., L234A or L234E. The L235 substitution can be, e.g., L235A or L235E. The G237 substitution can be, e.g., G237A. The D356 substitution can be, e.g., D356E. The L358 substitution can be, e.g., L358M. In some embodiments, an anti-MASP-2 antibody or antigen-binding fragment described herein comprises an IgG1 heavy chain constant region that comprises one or more amino acid substitutions selected from the group consisting of K214R, L234A, L235E, G237A, D356E, and L358M, numbered according to the EU Index. In some embodiments, the IgG1 heavy chain constant region comprises one or more amino acid substitutions selected from the group consisting of K214R, L234A, L235E, G237A, A330S, P331S, D356E, and L358M, numbered according to the EU Index. In some embodiments, the IgG1 heavy chain constant region comprises one or more amino acid substitutions selected from the group consisting of K214R, C226S, C229S, and P238S, numbered according to the EU Index. In some embodiments, the IgG1 heavy chain constant region comprises one or more amino acid substitutions selected from the group consisting of K214R, D356E, and L358M, numbered according to the EU Index. In some embodiments, the IgG1 heavy chain constant region comprises one or more amino acid substitutions selected from the group consisting of S131C, K133R, G137E, G138S, Q196K, 1199T, N203D, K214R, C226S, C229S, and P238S, numbered according to the EU Index.

In some embodiments, an anti-MASP-2 antibody or antigen-binding fragment described herein comprises an IgG4 heavy chain constant region that comprises one or more amino acid substitutions that increase the antibody or antigen-binding fragment's terminal half-life. As understood in the art, the terminal half-life, also referred to as the biological half-life, means the time required for plasma/blood concentration to decrease by 50% after administration and after pseudo-equilibrium of distribution has been reached. The one or more amino acid substitutions can be selected from the group consisting of S228, F234, L235, M252, S254, T256, K288, T307, M428, N434, H435, and Y436, numbered according to the EU Index. In some embodiments, the IgG4 heavy chain constant region has an amino acid substitution at S228. In some embodiments, the IgG4 heavy chain constant region has an amino acid substitution at F234. In some embodiments, the IgG4 heavy chain constant region has an amino acid substitution at L235. In some embodiments, the IgG4 heavy chain constant region has an amino acid substitution at M252. In some embodiments, the IgG4 heavy chain constant region has an amino acid substitution at S254. In some embodiments, the IgG4 heavy chain constant region has an amino acid substitution at T256. In some embodiments, the IgG4 heavy chain constant region has an amino acid substitution at K288. In some embodiments, the IgG4 heavy chain constant region has an amino acid substitution at T307. In some embodiments, the IgG4 heavy chain constant region has an amino acid substitution at M428. In some embodiments, the IgG4 heavy chain constant region has an amino acid substitution at N434. In some embodiments, the IgG4 heavy chain constant region has an amino acid substitution at H435. In some embodiments, the IgG4 heavy chain constant region has an amino acid substitution at Y436. The amino acid substitution at S228 can be, e.g., S228P. The amino acid substitution at F234 can be, e.g., F234A. The amino acid substitution at L235 can be, e.g., L235A. The amino acid substitution at M252 can be, e.g., M252Y. The amino acid substitution at S254 can be, e.g., S254T. The amino acid substitution at T256 can be, e.g., T256E. The amino acid substitution at K288 can be, e.g., K288A. The amino acid substitution at T307 can be, e.g., T307Q or T307H. The amino acid substitution at M428 can be, e.g., M428L. The amino acid substitution at N434 can be, e.g., N434A. The amino acid substitution at H435 can be, e.g., H435A or H435R. The amino acid substitution at Y436 can be, e.g., Y436T or Y436F.

In some embodiments, the anti-MASP-2 antibodies and antigen-binding fragments described herein comprises an IgG4 heavy chain constant region modified by amino acid substitutions selected from the group consisting of i) S228P; ii) F234A and L235A; iii) S228P, F234A, and L235A; (iv) T307H and N434A; v) M252Y, S254T and T256E; vi) M428L, N434A and Y436T; vii) S228P, M252Y, S254T and T256E; viii) S228P, F234A, L235A, M252Y, S254T and T256E; ix) S228P, F234A, L235A, T307Q, and N434A; and (x) M252Y, S254T, T307H and N434A.

In some embodiments, the anti-MASP-2 antibodies and antigen-binding fragments described herein comprise a variant of human IgG4 heavy chain modified by amino acid substitution S228P. In some embodiments, the anti-MASP-2 antibodies and antigen-binding fragments described herein comprise a variant of human IgG4 heavy chain modified by amino acid substitutions F234A and L235A. In some embodiments, the anti-MASP-2 antibodies and antigen-binding fragments described herein comprise a variant of human IgG4 heavy chain modified by amino acid substitutions S228P, F234A, and L235A. In some embodiments, the anti-MASP-2 antibodies and antigen-binding fragments described herein comprise a variant of human IgG4 heavy chain modified by amino acid substitutions T307H and N434A. In some embodiments, the anti-MASP-2 antibodies and antigen-binding fragments described herein comprise a variant of human IgG4 heavy chain modified by amino acid substitutions M252Y, S254T and T256E. In some embodiments, the anti-MASP-2 antibodies and antigen-binding fragments described herein comprise a variant of human IgG4 heavy chain modified by amino acid substitutions M428L, N434A and Y436T. In some embodiments, the anti-MASP-2 antibodies and antigen-binding fragments described herein comprise a variant of human IgG4 heavy chain modified by amino acid substitutions S228P, M252Y, S254T and T256E. In some embodiments, the anti-MASP-2 antibodies and antigen-binding fragments described herein comprise a variant of human IgG4 heavy chain modified by amino acid substitutions S228P, F234A, L235A, M252Y, S254T and T256E. In some embodiments, the anti-MASP-2 antibodies and antigen-binding fragments described herein comprise a variant of human IgG4 heavy chain modified by amino acid substitutions S228P, F234A, L235A, T307Q, and N434A. In some embodiments, the anti-MASP-2 antibodies and antigen-binding fragments described herein comprise a variant of human IgG4 heavy chain modified by amino acid substitutions M252Y, S254T, T307H and N434A.

In some embodiments, provided herein are anti-MASP-2 antibodies or antigen-binding fragments comprising a light chain having the amino acid sequence of SEQ ID NO: 33, or a variant thereof having an amino acid sequence that is at least about 80%, about 85%, about 90%, about 91%, about 92%, about 93%, about 94%, about 95%, about 96%, about 97%, about 98%, about 99% or 100% identical to SEQ ID NO: 33.

In some embodiments, provided herein are anti-MASP-2 antibodies or antigen-binding fragments comprising a heavy chain having the amino acid sequence of SEQ ID NO: 34, or a variant thereof having an amino acid sequence that is at least about 80%, about 85%, about 90%, about 91%, about 92%, about 93%, about 94%, about 95%, about 96%, about 97%, about 98%, about 99% or 100% identical to SEQ ID NO: 34. In some embodiments, provided herein are anti-MASP-2 antibodies or antigen-binding fragments comprising a heavy chain having an amino acid sequence selected from the group consisting of SEQ ID NOs: 34-74. In some embodiments, provided herein are anti-MASP-2 antibodies or antigen-binding fragments comprising a heavy chain having the amino acid sequence of SEQ ID NO: 34. In some embodiments, provided herein are anti-MASP-2 antibodies or antigen-binding fragments comprising a heavy chain having the amino acid sequence of SEQ ID NO: 35. In some embodiments, provided herein are anti-MASP-2 antibodies or antigen-binding fragments comprising a heavy chain having the amino acid sequence of SEQ ID NO: 36. In some embodiments, provided herein are anti-MASP-2 antibodies or antigen-binding fragments comprising a heavy chain having the amino acid sequence of SEQ ID NO: 37. In some embodiments, provided herein are anti-MASP-2 antibodies or antigen-binding fragments comprising a heavy chain having the amino acid sequence of SEQ ID NO: 38. In some embodiments, provided herein are anti-MASP-2 antibodies or antigen-binding fragments comprising a heavy chain having the amino acid sequence of SEQ ID NO: 39. In some embodiments, provided herein are anti-MASP-2 antibodies or antigen-binding fragments comprising a heavy chain having the amino acid sequence of SEQ ID NO: 40. In some embodiments, provided herein are anti-MASP-2 antibodies or antigen-binding fragments comprising a heavy chain having the amino acid sequence of SEQ ID NO: 41. In some embodiments, provided herein are anti-MASP-2 antibodies or antigen-binding fragments comprising a heavy chain having the amino acid sequence of SEQ ID NO: 42. In some embodiments, provided herein are anti-MASP-2 antibodies or antigen-binding fragments comprising a heavy chain having the amino acid sequence of SEQ ID NO: 43. In some embodiments, provided herein are anti-MASP-2 antibodies or antigen-binding fragments comprising a heavy chain having the amino acid sequence of SEQ ID NO: 44. In some embodiments, provided herein are anti-MASP-2 antibodies or antigen-binding fragments comprising a heavy chain having the amino acid sequence of SEQ ID NO: 45. In some embodiments, provided herein are anti-MASP-2 antibodies or antigen-binding fragments comprising a heavy chain having the amino acid sequence of SEQ ID NO: 46. In some embodiments, provided herein are anti-MASP-2 antibodies or antigen-binding fragments comprising a heavy chain having the amino acid sequence of SEQ ID NO: 47. In some embodiments, provided herein are anti-MASP-2 antibodies or antigen-binding fragments comprising a heavy chain having the amino acid sequence of SEQ ID NO: 48. In some embodiments, provided herein are anti-MASP-2 antibodies or antigen-binding fragments comprising a heavy chain having the amino acid sequence of SEQ ID NO: 49. In some embodiments, provided herein are anti-MASP-2 antibodies or antigen-binding fragments comprising a heavy chain having the amino acid sequence of SEQ ID NO: 50. In some embodiments, provided herein are anti-MASP-2 antibodies or antigen-binding fragments comprising a heavy chain having the amino acid sequence of SEQ ID NO: 51. In some embodiments, provided herein are anti-MASP-2 antibodies or antigen-binding fragments comprising a heavy chain having the amino acid sequence of SEQ ID NO: 52. In some embodiments, provided herein are anti-MASP-2 antibodies or antigen-binding fragments comprising a heavy chain having the amino acid sequence of SEQ ID NO: 53. In some embodiments, provided herein are anti-MASP-2 antibodies or antigen-binding fragments comprising a heavy chain having the amino acid sequence of SEQ ID NO: 54. In some embodiments, provided herein are anti-MASP-2 antibodies or antigen-binding fragments comprising a heavy chain having the amino acid sequence of SEQ ID NO: 55. In some embodiments, provided herein are anti-MASP-2 antibodies or antigen-binding fragments comprising a heavy chain having the amino acid sequence of SEQ ID NO: 56. In some embodiments, provided herein are anti-MASP-2 antibodies or antigen-binding fragments comprising a heavy chain having the amino acid sequence of SEQ ID NO: 57. In some embodiments, provided herein are anti-MASP-2 antibodies or antigen-binding fragments comprising a heavy chain having the amino acid sequence of SEQ ID NO: 58. In some embodiments, provided herein are anti-MASP-2 antibodies or antigen-binding fragments comprising a heavy chain having the amino acid sequence of SEQ ID NO: 59. In some embodiments, provided herein are anti-MASP-2 antibodies or antigen-binding fragments comprising a heavy chain having the amino acid sequence of SEQ ID NO: 60. In some embodiments, provided herein are anti-MASP-2 antibodies or antigen-binding fragments comprising a heavy chain having the amino acid sequence of SEQ ID NO: 61. In some embodiments, provided herein are anti-MASP-2 antibodies or antigen-binding fragments comprising a heavy chain having the amino acid sequence of SEQ ID NO: 62. In some embodiments, provided herein are anti-MASP-2 antibodies or antigen-binding fragments comprising a heavy chain having the amino acid sequence of SEQ ID NO: 63. In some embodiments, provided herein are anti-MASP-2 antibodies or antigen-binding fragments comprising a heavy chain having the amino acid sequence of SEQ ID NO: 64. In some embodiments, provided herein are anti-MASP-2 antibodies or antigen-binding fragments comprising a heavy chain having the amino acid sequence of SEQ ID NO: 65. In some embodiments, provided herein are anti-MASP-2 antibodies or antigen-binding fragments comprising a heavy chain having the amino acid sequence of SEQ ID NO: 66. In some embodiments, provided herein are anti-MASP-2 antibodies or antigen-binding fragments comprising a heavy chain having the amino acid sequence of SEQ ID NO: 67. In some embodiments, provided herein are anti-MASP-2 antibodies or antigen-binding fragments comprising a heavy chain having the amino acid sequence of SEQ ID NO: 68. In some embodiments, provided herein are anti-MASP-2 antibodies or antigen-binding fragments comprising a heavy chain having the amino acid sequence of SEQ ID NO: 69. In some embodiments, provided herein are anti-MASP-2 antibodies or antigen-binding fragments comprising a heavy chain having the amino acid sequence of SEQ ID NO: 70. In some embodiments, provided herein are anti-MASP-2 antibodies or antigen-binding fragments comprising a heavy chain having the amino acid sequence of SEQ ID NO: 71. In some embodiments, provided herein are anti-MASP-2 antibodies or antigen-binding fragments comprising a heavy chain having the amino acid sequence of SEQ ID NO: 72. In some embodiments, provided herein are anti-MASP-2 antibodies or antigen-binding fragments comprising a heavy chain having the amino acid sequence of SEQ ID NO: 73. In some embodiments, provided herein are anti-MASP-2 antibodies or antigen-binding fragments comprising a heavy chain having the amino acid sequence of SEQ ID NO: 74.

In some embodiments, provided herein are anti-MASP-2 antibodies or antigen-binding fragments comprising a light chain having the amino acid sequence of SEQ ID NO: 33, or a variant thereof having an amino acid sequence that is at least about 80%, about 85%, about 90%, about 91%, about 92%, about 93%, about 94%, about 95%, about 96%, about 97%, about 98%, about 99% or 100% identical to SEQ ID NO: 33 and a heavy chain having the amino acid sequence of SEQ ID NO: 34, or a variant thereof having an amino acid sequence that is at least about 80%, about 85%, about 90%, about 91%, about 92%, about 93%, about 94%, about 95%, about 96%, about 97%, about 98%, about 99% or 100% identical to SEQ ID NO: 34.

In some embodiments, provided herein are anti-MASP-2 antibodies or antigen-binding fragments comprising a light chain and a heavy chain having the amino acid sequences of SEQ ID NOs: 33 and 34, respectively. In some embodiments, provided herein are anti-MASP-2 antibodies or antigen-binding fragments comprising a light chain and a heavy chain having the amino acid sequences of SEQ ID NOs: 33 and 35, respectively. In some embodiments, provided herein are anti-MASP-2 antibodies or antigen-binding fragments comprising a light chain and a heavy chain having the amino acid sequences of SEQ ID NOs: 33 and 36, respectively. In some embodiments, provided herein are anti-MASP-2 antibodies or antigen-binding fragments comprising a light chain and a heavy chain having the amino acid sequences of SEQ ID NOs: 33 and 37, respectively. In some embodiments, provided herein are anti-MASP-2 antibodies or antigen-binding fragments comprising a light chain and a heavy chain having the amino acid sequences of SEQ ID NOs: 33 and 38, respectively. In some embodiments, provided herein are anti-MASP-2 antibodies or antigen-binding fragments comprising a light chain and a heavy chain having the amino acid sequences of SEQ ID NOs: 33 and 39, respectively. In some embodiments, provided herein are anti-MASP-2 antibodies or antigen-binding fragments comprising a light chain and a heavy chain having the amino acid sequences of SEQ ID NOs: 33 and 40, respectively. In some embodiments, provided herein are anti-MASP-2 antibodies or antigen-binding fragments comprising a light chain and a heavy chain having the amino acid sequences of SEQ ID NOs: 33 and 41, respectively. In some embodiments, provided herein are anti-MASP-2 antibodies or antigen-binding fragments comprising a light chain and a heavy chain having the amino acid sequences of SEQ ID NOs: 33 and 42, respectively. In some embodiments, provided herein are anti-MASP-2 antibodies or antigen-binding fragments comprising a light chain and a heavy chain having the amino acid sequences of SEQ ID NOs: 33 and 43, respectively. In some embodiments, provided herein are anti-MASP-2 antibodies or antigen-binding fragments comprising a light chain and a heavy chain having the amino acid sequences of SEQ ID NOs: 33 and 44, respectively. In some embodiments, provided herein are anti-MASP-2 antibodies or antigen-binding fragments comprising a light chain and a heavy chain having the amino acid sequences of SEQ ID NOs: 33 and 45, respectively. In some embodiments, provided herein are anti-MASP-2 antibodies or antigen-binding fragments comprising a light chain and a heavy chain having the amino acid sequences of SEQ ID NOs: 33 and 46, respectively. In some embodiments, provided herein are anti-MASP-2 antibodies or antigen-binding fragments comprising a light chain and a heavy chain having the amino acid sequences of SEQ ID NOs: 33 and 47, respectively. In some embodiments, provided herein are anti-MASP-2 antibodies or antigen-binding fragments comprising a light chain and a heavy chain having the amino acid sequences of SEQ ID NOs: 33 and 48, respectively. In some embodiments, provided herein are anti-MASP-2 antibodies or antigen-binding fragments comprising a light chain and a heavy chain having the amino acid sequences of SEQ ID NOs: 33 and 49, respectively. In some embodiments, provided herein are anti-MASP-2 antibodies or antigen-binding fragments comprising a light chain and a heavy chain having the amino acid sequences of SEQ ID NOs: 33 and 50, respectively. In some embodiments, provided herein are anti-MASP-2 antibodies or antigen-binding fragments comprising a light chain and a heavy chain having the amino acid sequences of SEQ ID NOs: 33 and 51, respectively. In some embodiments, provided herein are anti-MASP-2 antibodies or antigen-binding fragments comprising a light chain and a heavy chain having the amino acid sequences of SEQ ID NOs: 33 and 52, respectively. In some embodiments, provided herein are anti-MASP-2 antibodies or antigen-binding fragments comprising a light chain and a heavy chain having the amino acid sequences of SEQ ID NOs: 33 and 53, respectively. In some embodiments, provided herein are anti-MASP-2 antibodies or antigen-binding fragments comprising a light chain and a heavy chain having the amino acid sequences of SEQ ID NOs: 33 and 54, respectively. In some embodiments, provided herein are anti-MASP-2 antibodies or antigen-binding fragments comprising a light chain and a heavy chain having the amino acid sequences of SEQ ID NOs: 33 and 55, respectively. In some embodiments, provided herein are anti-MASP-2 antibodies or antigen-binding fragments comprising a light chain and a heavy chain having the amino acid sequences of SEQ ID NOs: 33 and 56, respectively. In some embodiments, provided herein are anti-MASP-2 antibodies or antigen-binding fragments comprising a light chain and a heavy chain having the amino acid sequences of SEQ ID NOs: 33 and 57, respectively. In some embodiments, provided herein are anti-MASP-2 antibodies or antigen-binding fragments comprising a light chain and a heavy chain having the amino acid sequences of SEQ ID NOs: 33 and 58, respectively. In some embodiments, provided herein are anti-MASP-2 antibodies or antigen-binding fragments comprising a light chain and a heavy chain having the amino acid sequences of SEQ ID NOs: 33 and 59, respectively. In some embodiments, provided herein are anti-MASP-2 antibodies or antigen-binding fragments comprising a light chain and a heavy chain having the amino acid sequences of SEQ ID NOs: 33 and 60, respectively. In some embodiments, provided herein are anti-MASP-2 antibodies or antigen-binding fragments comprising a light chain and a heavy chain having the amino acid sequences of SEQ ID NOs: 33 and 61, respectively. In some embodiments, provided herein are anti-MASP-2 antibodies or antigen-binding fragments comprising a light chain and a heavy chain having the amino acid sequences of SEQ ID NOs: 33 and 62, respectively. In some embodiments, provided herein are anti-MASP-2 antibodies or antigen-binding fragments comprising a light chain and a heavy chain having the amino acid sequences of SEQ ID NOs: 33 and 63, respectively. In some embodiments, provided herein are anti-MASP-2 antibodies or antigen-binding fragments comprising a light chain and a heavy chain having the amino acid sequences of SEQ ID NOs: 33 and 64, respectively. In some embodiments, provided herein are anti-MASP-2 antibodies or antigen-binding fragments comprising a light chain and a heavy chain having the amino acid sequences of SEQ ID NOs: 33 and 65, respectively. In some embodiments, provided herein are anti-MASP-2 antibodies or antigen-binding fragments comprising a light chain and a heavy chain having the amino acid sequences of SEQ ID NOs: 33 and 66, respectively. In some embodiments, provided herein are anti-MASP-2 antibodies or antigen-binding fragments comprising a light chain and a heavy chain having the amino acid sequences of SEQ ID NOs: 33 and 67, respectively. In some embodiments, provided herein are anti-MASP-2 antibodies or antigen-binding fragments comprising a light chain and a heavy chain having the amino acid sequences of SEQ ID NOs: 33 and 68, respectively. In some embodiments, provided herein are anti-MASP-2 antibodies or antigen-binding fragments comprising a light chain and a heavy chain having the amino acid sequences of SEQ ID NOs: 33 and 69, respectively. In some embodiments, provided herein are anti-MASP-2 antibodies or antigen-binding fragments comprising a light chain and a heavy chain having the amino acid sequences of SEQ ID NOs: 33 and 70, respectively. In some embodiments, provided herein are anti-MASP-2 antibodies or antigen-binding fragments comprising a light chain and a heavy chain having the amino acid sequences of SEQ ID NOs: 33 and 71, respectively. In some embodiments, provided herein are anti-MASP-2 antibodies or antigen-binding fragments comprising a light chain and a heavy chain having the amino acid sequences of SEQ ID NOs: 33 and 72, respectively. In some embodiments, provided herein are anti-MASP-2 antibodies or antigen-binding fragments comprising a light chain and a heavy chain having the amino acid sequences of SEQ ID NOs: 33 and 73, respectively. In some embodiments, provided herein are anti-MASP-2 antibodies or antigen-binding fragments comprising a light chain and a heavy chain having the amino acid sequences of SEQ ID NOs: 33 and 74, respectively.

In some embodiments, variants can include addition of amino acid residues at the amino- and/or carboxyl-terminal end of the antibody or polypeptide. The length of additional amino acids residues can range from one residue to a hundred or more residues. In some embodiments, a variant comprises an N-terminal methionyl residue. In some embodiments, the variant comprises an additional polypeptide/protein (e.g., Fc region) to create a fusion protein. In some embodiments, a variant is engineered to be detectable and may comprise a detectable label and/or protein (e.g., a fluorescent tag or an enzyme).

The variant antibodies or antigen-binding fragments described herein can be generated using methods known in the art, including but not limited to, site-directed mutagenesis, alanine scanning mutagenesis, and PCR mutagenesis.

In some embodiments, a variant of an anti-MASP-2 antibody or antigen-binding fragment disclosed herein can retain the ability to bind MASP-2 to a similar extent, the same extent, or to a higher extent, as the parent antibody or antigen-binding fragment. In some embodiments, the variant can be at least about 80%, about 85%, about 90%, about 91%, about 92%, about 93%, about 94%, about 95%, about 96%, about 97%, about 98%, about 99% or more identical in amino acid sequence to the parent antibody or antigen-binding fragment. In certain embodiments, a variant of an anti-MASP-2 antibody or antigen-binding fragment comprises the amino acid sequence of the parent anti-MASP-2 antibody or antigen-binding fragment with one or more conservative amino acid substitution. Conservative amino acid substitutions are known in the art and include amino acid substitutions in which one amino acid having certain physical and/or chemical properties is exchanged for another amino acid that has the same or similar chemical or physical properties.

In some embodiments, a variant of an anti-MASP-2 antibody or antigen-binding fragment comprises the amino acid sequence of the parent antibody or antigen-binding fragment with one or more non-conservative amino acid substitutions. In some embodiments, a variant of an anti-MASP-2 antibody or antigen-binding fragment comprises the amino acid sequence of the parent binding antibody or antigen-binding fragment with one or more non-conservative amino acid substitution, wherein the one or more non-conservative amino acid substitutions do not interfere with or inhibit one or more biological activities of the variant (e.g., MASP-2 binding). In certain embodiments, the one or more conservative amino acid substitutions and/or the one or more non-conservative amino acid substitutions can enhance a biological activity of the variant, such that the biological activity of the functional variant is increased as compared to the parent binding moiety.

In some embodiments, the variant can have 1, 2, 3, 4, or 5 amino acid substitutions in the CDRs (e.g., VH CDR1, VH CDR2, VH CDR3, VL CDR1, VL CDR2 and VL CDR3) of the binding moiety.

In some embodiments, anti-MASP-2 antibodies or antigen-binding fragments described herein are chemically modified naturally or by intervention. In some embodiments, the anti-MASP-2 antibodies or antigen-binding fragments have been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications can be carried out by known techniques. The anti-MASP-2 antibodies or antigen-binding fragments can comprise one or more analogs of an amino acid (including, for example, unnatural amino acids), as well as other modifications known in the art.

Epitope mapping is a method of identifying the binding site, region, or epitope on a target protein where an antibody binds. A variety of methods are known in the art for mapping epitopes on target proteins. These methods include mutagenesis, including but not limited to, shotgun mutagenesis, site-directed mutagenesis, and alanine scanning; domain or fragment scanning; peptide scanning (e.g., Pepscan technology); display methods (e.g., phage display, microbial display, and ribosome/mRNA display); methods involving proteolysis and mass spectroscopy; and structural determination (e.g., X-ray crystallography and NMR). In some embodiments, anti-MASP-2 antibodies or antigen-binding fragments described herein are characterized by assays including, but not limited to, N-terminal sequencing, amino acid analysis, HPLC, mass spectrometry, ion exchange chromatography, and papain digestion.

As provided in further detail in Experimental section below, antibodies 3E10 and humanized 3E10 as provided herein bind to the protease domain of human MASP-2 (amino acid 415-671 residues of SEQ ID NO: 1), and do not compete for binding to human MASP-2 with benchmark antibody narsoplimab. In some embodiments, provides herein are anti-MASP-2 antibodies and antigen-binding fragments that bind to the protease domain of human MASP-2. In some embodiments, the anti-MASP-2 antibodies and antigen-binding fragments provided herein do not detectably bind to a truncated human MASP-2 that lack the protease domain. In some embodiments, the anti-MASP-2 antibodies and antigen-binding fragments provided herein do not compete for human MASP-2 binding with narsoplimab.

The anti-MASP-2 antibodies or antigen-binding fragments of the present disclosure can be analyzed for their physical, chemical and/or biological properties by various methods known in the art. In some embodiments, an anti-MASP-2 antibody is tested for its ability to bind MASP-2 (e.g., human MASP-2). Binding assays include, but are not limited to, BLI, SPR (e.g., Biacore), ELISA, and FACS. In addition, antibodies can be evaluated for solubility, stability, thermostability, viscosity, expression levels, expression quality, and/or purification efficiency.

In some embodiments, anti-MASP-2 antibodies or antigen-binding fragments described herein bind to human MASP-2 with high affinity, for example, with a K_{D} of 10⁻⁷ M or less, 10⁻⁸ M or less, 5×10⁻⁹ M or less, 10⁻⁹ M or less, 5×10⁻¹⁰ M or less, 10⁻¹⁰ M or less, 5×10⁻¹¹ M or less, 10⁻¹¹ M or less, 5×10⁻¹² M or less, or 10⁻¹² M or less; or ranging from 10⁻¹² M to 10⁻⁷ M, from 10⁻¹¹ M to 10⁻⁷ M, from 10⁻¹⁰ M to 10⁻⁷ M, from 10⁻⁹ M to 10⁻⁷ M, from 10⁻⁸ M to 10⁻⁷ M, from 10⁻¹⁰ M to 10⁻⁸ M, from 10⁻⁹ M to 10⁻⁸ M, from 10⁻¹¹ M to 10⁻⁹ M, or from 10⁻¹⁰ M to 10⁻⁹ M. In some embodiments, the K_{D} is determined by BLI. In some embodiments, the K_{D} is determined by SPR. In some embodiments, anti-MASP-2 antibodies or antigen-binding fragments described herein bind to human MASP-2 with high affinity, for example, with a K_{D} of 10⁻⁷ M or less, 10⁻⁸ M or less, 5×10⁻⁹ M or less, 10⁻⁹ M or less, 5×10⁻¹⁰ M or less, 10⁻¹⁰ M or less, 5×10⁻¹¹ M or less, 10⁻¹¹ M or less, 5×10⁻¹² M or less, or 10⁻¹² M or less; or ranging from 10⁻¹² M to 10⁻⁷ M, from 10⁻¹¹ M to 10⁻⁷ M, from 10⁻¹⁰ M to 10⁻⁷ M, from 10⁻⁹ M to 10⁻⁷ M, from 10⁻⁸ M to 10⁻⁷ M, from 10⁻¹⁰ M to 10⁻⁸ M, from 10⁻⁹ M to 10⁻⁸ M, from 10⁻¹¹ M to 10⁻⁹ M, or from 10⁻¹⁰ M to 10⁻⁹ M, as measured by SPR.

In some embodiments, anti-MASP-2 antibodies or antigen-binding fragments described herein bind to human MASP-2 with a K_{D} of that is 10⁻⁹ M or less, as measured by SPR. In some embodiments, anti-MASP-2 antibodies or antigen-binding fragments described herein bind to human MASP-2 with a K_{D} of 10⁻¹⁰ M or less, as measured by SPR. In some embodiments, anti-MASP-2 antibodies or antigen-binding fragments described herein bind to human MASP-2 with a K_{D} ranging from 10⁻¹¹ M to 10−09 M as measured by SPR. In some embodiments, anti-MASP-2 antibodies or antigen-binding fragments described herein bind to human MASP-2 with a K_{D} ranging from 5×10⁻¹¹ M to 10−09 M as measured by SPR. In some embodiments, anti-MASP-2 antibodies or antigen-binding fragments described herein bind to human MASP-2 with a K_{D} ranging from 5×10⁻¹¹ M to 5×10⁻¹⁰ M as measured by SPR. In some embodiments, anti-MASP-2 antibodies or antigen-binding fragments described herein bind to human MASP-2 with a K_{D} of about 10⁻¹⁰ M as measured by SPR.

The anti-MASP-2 antibodies and antigen-binding fragments described herein can inhibit MASP-2-dependnet complement activation. In other words, the anti-MASP-2 antibodies and antigen-binding fragments described herein can inhibit the lectin pathway of completement activation. In some embodiments, the anti-MASP-2 antibodies or antigen-binding fragments provided herein can inhibit MASP-2-dependnet complement activation by at least 20%, at least 30%, at least 40%, at least 50%, at least 60%, at least 70%, at least 80%, or at least 90%. In one embodiment, the anti-MASP-2 antibodies or antigen-binding fragments provided herein reduce MASP-2-dependent complement activation by at least 50% (i.e., resulting in MASP-2-dependent complement activation of 50% or less as compared to the activation level in the absence of the antibody or antigen-binding fragment). In one embodiment, the anti-MASP-2 antibodies or antigen-binding fragments provided herein reduce MASP-2-dependent complement activation by at least 80% (i.e., resulting in MASP-2-dependent complement activation of 20% or less as compared to the activation level in the absence of the antibody or antigen-binding fragment). In one embodiment, the anti-MASP-2 antibodies or antigen-binding fragments provided herein reduce MASP-2-dependent complement activation by at least 90% (i.e., resulting in MASP-2-dependent complement activation of 10% or less as compared to the activation level in the absence of the antibody or antigen-binding fragment).

In some embodiments, the anti-MASP-2 antibodies and antigen-binding fragments described herein do not inhibit the classical pathway or alternative pathway of complement activation.

The inhibitory effects of anti-MASP-2 antibodies and antigen-binding fragments on MASP-2-dependent complement activation provided herein can be measured at multiple levels, such as C4 activation, C3 activation, and MAC activation. Various forms of assays are known in the art to measure, for example, C4 activation, C3 activation, and MAC activation (see e.g., WO2012/151481); some are described in the example sections below.

The anti-MASP-2 antibodies and antigen-binding fragments described herein can inhibit C4 activation. In some embodiments, anti-MASP-2 antibodies and antigen-binding fragments described herein can inhibit C4 activation, e.g., as measured in in vitro assay, with an IC₅₀ of 0.5 μg/mL or less, 0.4 μg/mL or less, 0.3 μg/mL or less, 0.2 μg/mL or less, 0.1 μg/mL or less, 0.05 μg/mL or less, 0.04 μg/mL or less, 0.03 μg/mL or less, 0.02 μg/mL or less, 0.01 μg/mL or less, 0.005 μg/mL or less, or 0.001 μg/mL or less. In some embodiments, anti-MASP-2 antibodies and antigen-binding fragments described herein can inhibit C4 activation, e.g., as measured in in vitro assay, with an IC₅₀ of about 0.2 μg/mL or less. In some embodiments, anti-MASP-2 antibodies and antigen-binding fragments described herein can inhibit C4 activation, e.g., as measured in in vitro assay, with an IC₅₀ of about 0.1 μg/mL or less. In some embodiments, anti-MASP-2 antibodies and antigen-binding fragments described herein can inhibit C4 activation, e.g., as measured in in vitro assay, with an IC₅₀ of about 0.05 μg/mL or less. In some embodiments, anti-MASP-2 antibodies and antigen-binding fragments described herein can inhibit C4 activation, e.g., as measured in in vitro assay, with an IC₅₀ of about 0.01 μg/mL or less. In some embodiments, anti-MASP-2 antibodies and antigen-binding fragments described herein can inhibit C4 activation, e.g., as measured in in vitro assay, with an IC₅₀ ranging 0.001-0.5 μg/mL, 0.001-0.2 μg/mL, 0.001-0.1 μg/mL, 0.001-0.05 μg/mL, 0.001-0.01 μg/mL, 0.001-0.005 μg/mL, 0.005-0.5 μg/mL, 0.005-0.2 μg/mL, 0.005-0.1 μg/mL, 0.005-0.05 μg/mL, 0.005-0.01 μg/mL, or 0.001-0.5 μg/mL. In some embodiments, anti-MASP-2 antibodies and antigen-binding fragments described herein can inhibit C4 activation, e.g., as measured in in vitro assay, with an IC₅₀ ranging 0.001-0.1 μg/mL. In some embodiments, anti-MASP-2 antibodies and antigen-binding fragments described herein can inhibit C4 activation, e.g., as measured in in vitro assay, with an IC₅₀ ranging 0.001-0.01 μg/mL.

The C4 activation assay can be performed in the presence in various concentrations of human serum (e.g., 1-100% or 5-50%) to measure the inhibitory effect of an anti-MASP-2 antibody or antigen-binding fragment. In some embodiments, anti-MASP-2 antibodies and antigen-binding fragments described herein can inhibit C4 activation, e.g., as measured in in vitro assay, with an IC₅₀ of 0.5 μg/mL or less, 0.4 μg/mL or less, 0.3 μg/mL or less, 0.2 μg/mL or less, 0.1 μg/mL or less, 0.05 μg/mL or less, 0.04 μg/mL or less, 0.03 μg/mL or less, 0.02 μg/mL or less, 0.01 μg/mL or less, 0.005 μg/mL or less, or 0.001 μg/mL or less in the presence of about 5%, about 10%, about 20%, about 30%, about 40%, about 50%, about 60%, about 70%, about 80%, or about 90% human serum. In some embodiments, anti-MASP-2 antibodies and antigen-binding fragments described herein can inhibit C4 activation, e.g., as measured in in vitro assay, with an IC₅₀ of 0.5 μg/mL or less, 0.4 μg/mL or less, 0.3 μg/mL or less, 0.2 μg/mL or less, 0.1 μg/mL or less, 0.05 μg/mL or less, 0.04 μg/mL or less, 0.03 μg/mL or less, 0.02 μg/mL or less, 0.01 μg/mL or less, 0.005 μg/mL or less, or 0.001 μg/mL or less in the presence of about 5% human serum. In some embodiments, anti-MASP-2 antibodies and antigen-binding fragments described herein can inhibit C4 activation, e.g., as measured in in vitro assay, with an IC₅₀ of 0.5 μg/mL or less, 0.4 μg/mL or less, 0.3 μg/mL or less, 0.2 μg/mL or less, 0.1 μg/mL or less, 0.05 μg/mL or less, 0.04 μg/mL or less, 0.03 μg/mL or less, 0.02 μg/mL or less, 0.01 μg/mL or less, 0.005 μg/mL or less, or 0.001 μg/mL or less in the presence of about 10% human serum. In some embodiments, anti-MASP-2 antibodies and antigen-binding fragments described herein can inhibit C4 activation, e.g., as measured in in vitro assay, with an IC₅₀ of 0.5 μg/mL or less, 0.4 μg/mL or less, 0.3 μg/mL or less, 0.2 μg/mL or less, 0.1 μg/mL or less, 0.05 μg/mL or less, 0.04 μg/mL or less, 0.03 μg/mL or less, 0.02 μg/mL or less, 0.01 μg/mL or less, 0.005 μg/mL or less, or 0.001 μg/mL or less in the presence of about 50% human serum.

In some embodiments, anti-MASP-2 antibodies and antigen-binding fragments described herein can inhibit C4 activation, e.g., as measured in in vitro assay, with an IC₅₀ of 0.2 μg/mL or less in the presence of about 5%, about 10%, about 20%, about 30%, about 40%, about 50%, about 60%, about 70%, about 80%, or about 90% human serum. In some embodiments, anti-MASP-2 antibodies and antigen-binding fragments described herein can inhibit C4 activation, e.g., as measured in in vitro assay, with an IC₅₀ of 0.1 μg/mL or less in the presence of about 5%, about 10%, about 20%, about 30%, about 40%, about 50%, about 60%, about 70%, about 80%, or about 90% human serum. In some embodiments, anti-MASP-2 antibodies and antigen-binding fragments described herein can inhibit C4 activation, e.g., as measured in in vitro assay, with an IC₅₀ of 0.05 μg/mL or less in the presence of about 5%, about 10%, about 20%, about 30%, about 40%, about 50%, about 60%, about 70%, about 80%, or about 90% human serum.

In some embodiments, anti-MASP-2 antibodies and antigen-binding fragments described herein can inhibit C4 activation, e.g., as measured in in vitro assay, in the presence of 1-100% human serum, with an IC₅₀ ranging 0.001-0.5 μg/mL, 0.001-0.2 μg/mL, 0.001-0.1 μg/mL, 0.001-0.05 μg/mL, 0.001-0.01 μg/mL, 0.001-0.005 μg/mL, 0.005-0.5 μg/mL, 0.005-0.2 μg/mL, 0.005-0.1 μg/mL, 0.005-0.05 μg/mL, 0.005-0.01 μg/mL, or 0.001-0.5 μg/mL in the presence of about 5%, about 10%, about 20%, about 30%, about 40%, about 50%, about 60%, about 70%, about 80%, or about 90% human serum. In some embodiments, anti-MASP-2 antibodies and antigen-binding fragments described herein can inhibit C4 activation, e.g., as measured in in vitro assay, with an IC₅₀ ranging 0.001-0.1 μg/mL in the presence of about 5%, about 10%, about 20%, about 30%, about 40%, about 50%, about 60%, about 70%, about 80%, or about 90% human serum. In some embodiments, anti-MASP-2 antibodies and antigen-binding fragments described herein can inhibit C4 activation, e.g., as measured in in vitro assay, with an IC₅₀ ranging 0.01-0.1 μg/mL in the presence of about 5%, about 10%, about 20%, about 30%, about 40%, about 50%, about 60%, about 70%, about 80%, or about 90% human serum.

In some embodiments, anti-MASP-2 antibodies and antigen-binding fragments described herein can inhibit C4 activation, e.g., as measured in in vitro assay, with an IC₅₀ ranging 0.001-0.1 μg/mL in the presence of about 5% human serum. In some embodiments, anti-MASP-2 antibodies and antigen-binding fragments described herein can inhibit C4 activation, e.g., as measured in in vitro assay, with an IC₅₀ ranging 0.01-0.1 μg/mL in the presence of about 5% human serum. In some embodiments, anti-MASP-2 antibodies and antigen-binding fragments described herein can inhibit C4 activation, e.g., as measured in in vitro assay, with an IC₅₀ ranging 0.001-0.1 μg/mL in the presence of about 10% human serum. In some embodiments, anti-MASP-2 antibodies and antigen-binding fragments described herein can inhibit C4 activation, e.g., as measured in in vitro assay, with an IC₅₀ ranging 0.01-0.1 μg/mL in the presence of about 10% human serum. In some embodiments, anti-MASP-2 antibodies and antigen-binding fragments described herein can inhibit C4 activation, e.g., as measured in in vitro assay, with an IC₅₀ ranging 0.001-0.1 μg/mL in the presence of about 50% human serum. In some embodiments, anti-MASP-2 antibodies and antigen-binding fragments described herein can inhibit C4 activation, e.g., as measured in in vitro assay, with an IC₅₀ ranging 0.01-0.1 μg/mL in the presence of about 50% human serum.

The anti-MASP-2 antibodies and antigen-binding fragments described herein can inhibit C3 activation. In some embodiments, anti-MASP-2 antibodies and antigen-binding fragments described herein can inhibit C3 activation, e.g., as measured in in vitro assay, with an IC₅₀ of 0.5 μg/mL or less, 0.4 μg/mL or less, 0.3 μg/mL or less, 0.2 μg/mL or less, 0.1 μg/mL or less, 0.05 μg/mL or less, 0.04 μg/mL or less, 0.03 μg/mL or less, 0.02 μg/mL or less, 0.01 μg/mL or less, 0.005 μg/mL or less, 0.002 μg/mL or less, or 0.001 μg/mL or less. In some embodiments, anti-MASP-2 antibodies and antigen-binding fragments described herein can inhibit C3 activation, e.g., as measured in in vitro assay, with an IC₅₀ of 0.2 μg/mL or less. In some embodiments, anti-MASP-2 antibodies and antigen-binding fragments described herein can inhibit C3 activation, e.g., as measured in in vitro assay, with an IC₅₀ of 0.1 μg/mL or less. In some embodiments, anti-MASP-2 antibodies and antigen-binding fragments described herein can inhibit C3 activation, e.g., as measured in in vitro assay, with an IC₅₀ of 0.05 μg/mL or less. In some embodiments, anti-MASP-2 antibodies and antigen-binding fragments described herein can inhibit C3 activation, e.g., as measured in in vitro assay, with an IC₅₀ ranging 0.001-0.5 μg/mL, 0.001-0.2 μg/mL, 0.001-0.1 μg/mL, 0.001-0.05 μg/mL, 0.001-0.01 μg/mL, 0.001-0.005 μg/mL, 0.005-0.5 μg/mL, 0.005-0.2 μg/mL, 0.005-0.1 μg/mL, 0.005-0.05 μg/mL, 0.005-0.01 μg/mL, or 0.001-0.5 μg/mL. In some embodiments, anti-MASP-2 antibodies and antigen-binding fragments described herein can inhibit C3 activation, e.g., as measured in in vitro assay, with an IC₅₀ ranging 0.001-0.1 μg/mL. In some embodiments, anti-MASP-2 antibodies and antigen-binding fragments described herein can inhibit C3 activation, e.g., as measured in in vitro assay, with an IC₅₀ ranging 0.001-0.05 μg/mL.

The anti-MASP-2 antibodies and antigen-binding fragments described herein can inhibit MAC activation. In some embodiments, anti-MASP-2 antibodies and antigen-binding fragments described herein can inhibit MAC activation, e.g., as measured in in vitro assay, with an IC₅₀ of 0.5 μg/mL or less, 0.4 μg/mL or less, 0.3 μg/mL or less, 0.2 μg/mL or less, 0.1 μg/mL or less, 0.05 μg/mL or less, 0.04 μg/mL or less, 0.03 μg/mL or less, 0.02 μg/mL or less, 0.01 μg/mL or less, 0.005 μg/mL or less, 0.002 μg/mL or less, or 0.001 μg/mL or less. In some embodiments, anti-MASP-2 antibodies and antigen-binding fragments described herein can inhibit MAC activation, e.g., as measured in in vitro assay, with an IC₅₀ of 0.2 μg/mL or less. In some embodiments, anti-MASP-2 antibodies and antigen-binding fragments described herein can inhibit MAC activation, e.g., as measured in in vitro assay, with an IC₅₀ of 0.1 μg/mL or less. In some embodiments, anti-MASP-2 antibodies and antigen-binding fragments described herein can inhibit MAC activation, e.g., as measured in in vitro assay, with an IC₅₀ of 0.05 μg/mL or less. In some embodiments, anti-MASP-2 antibodies and antigen-binding fragments described herein can inhibit MAC activation, e.g., as measured in in vitro assay, with an IC₅₀ ranging 0.001-0.5 μg/mL, 0.001-0.2 μg/mL, 0.001-0.1 μg/mL, 0.001-0.05 μg/mL, 0.001-0.01 μg/mL, 0.001-0.005 μg/mL, 0.005-0.5 μg/mL, 0.005-0.2 μg/mL, 0.005-0.1 μg/mL, 0.005-0.05 μg/mL, 0.005-0.01 μg/mL, or 0.001-0.5 μg/mL. In some embodiments, anti-MASP-2 antibodies and antigen-binding fragments described herein can inhibit MAC activation, e.g., as measured in in vitro assay, with an IC₅₀ ranging 0.001-0.1 μg/mL. In some embodiments, anti-MASP-2 antibodies and antigen-binding fragments described herein can inhibit MAC activation, e.g., as measured in in vitro assay, with an IC₅₀ ranging 0.001-0.05 μg/mL. In some embodiments, anti-MASP-2 antibodies and antigen-binding fragments described herein can inhibit MAC activation, e.g., as measured in in vitro assay, with an IC₅₀ ranging 0.001-0.01 μg/mL.

As such, in some embodiments, the anti-MASP-2 antibodies or antigen-binding fragments thereof described herein can (1) binds human MASP-2 with a K_{D} that is 1 nM or less, measured by SPR; (2) blocks C4 activation with an IC₅₀ of 0.1 μg/mL or less, measured in vitro; (3) blocks C3 activation with an IC₅₀ of 0.1 μg/mL or less, measured in vitro; (4) blocks MAC activation with an IC₅₀ of 0.1 μg/mL or less, measured in vitro; (5) does not affect the classical or alternative pathway of completement activation; or (6) any combination of (1)-(5).

In some embodiments, the anti-MASP-2 antibodies or antigen-binding fragments thereof described herein can (1) binds human MASP-2 with a K_{D} that is 1 nM or less, measured by SPR; and (2) blocks C4 activation with an IC₅₀ of 0.1 μg/mL or less, measured in vitro.

In some embodiments, provided herein are antibodies or antigen-binding fragments thereof that specifically bind the protease domain of human MASP-2, wherein the antibody or antigen binding fragment: (1) binds human MASP-2 with a K_{D} that is 1 nM or less, measured by SPR;
(2) blocks C4 activation with an IC₅₀ of 0.1 μg/mL or less, measured in vitro;
(3) blocks C3 activation with an IC₅₀ of 0.1 μg/mL or less, measured in vitro;
(4) blocks MAC activation with an IC₅₀ of 0.1 μg/mL or less, measured in vitro;
(5) does not affect the classical or alternative pathway of completement activation; or
(6) any combination of (1)-(5).

In some embodiments, provided herein are antibodies or antigen-binding fragments thereof that specifically bind the protease domain of human MASP-2, wherein the antibody or antigen binding fragment binds human MASP-2 with a K_{D} that is 1 nM or less, measured by SPR. In some embodiments, provided herein are antibodies or antigen-binding fragments thereof that specifically bind the protease domain of human MASP-2, wherein the antibodies or antigen binding fragments block C4 activation with an IC₅₀ of 0.1 μg/mL or less, measured in vitro. In some embodiments, provided herein are antibodies or antigen-binding fragments thereof that specifically bind the protease domain of human MASP-2, wherein the antibodies or antigen binding fragments block C3 activation with an IC₅₀ of 0.1 μg/mL or less, measured in vitro. In some embodiments, provided herein are antibodies or antigen-binding fragments thereof that specifically bind the protease domain of human MASP-2, wherein the antibodies or antigen binding fragments block MAC activation with an IC₅₀ of 0.1 μg/mL or less, measured in vitro. In some embodiments, the antibodies or antigen binding fragments do not affect the classical or alternative pathway of completement activation.

In some embodiments, an anti-MASP-2 antibody or antigen-binding fragment described herein is conjugated to a detectable substance or molecule that allows the agent to be used for diagnosis and/or detection. A detectable substance can include, but is not limited to, enzymes, such as horseradish peroxidase, alkaline phosphatase, beta-galactosidase, and acetylcholinesterase; prosthetic groups, such as biotin and flavine(s); fluorescent materials, such as, umbelliferone, fluorescein, fluorescein isothiocyanate (FITC), rhodamine, tetramethylrhodamine isothiocyanate (TRITC), dichlorotriazinylamine fluorescein, dansyl chloride, cyanine (Cy3), and phycoerythrin; bioluminescent materials, such as luciferase; radioactive materials, such as ²¹²Bi, ¹⁴C, ⁵⁷Co, ⁵¹Cr, ⁶⁷Cu, ¹⁸F, ⁶⁸Ga, ⁶⁷Ga, ¹⁵³ Gd, ¹⁵⁹Gd, ⁶⁸Ge, ³H, ¹⁶⁶Ho, ¹³¹I, ¹²⁵I, ¹²³I, ¹²¹I, ¹¹⁵In, ¹¹³In, ¹¹²In, ¹⁴⁰La, ¹⁷⁷Lu, ⁵⁴Mn, ⁹⁹Mo, ³²P, ¹⁰³Pd, ¹⁴⁹Pm, ¹⁴²Pr, ¹⁸⁶Re, ¹⁸⁸Re, ¹⁰⁵Rh, ⁹⁷Ru, ³⁵S, ⁴⁷Sc, ⁷⁵Se, ¹⁵³Sm, ¹¹³Sn, ¹¹⁷Sn, ⁸⁵Sr, ^{99m}Tc, ²⁰¹Ti, ¹³³Xe, ⁹⁰Y, ⁶⁹Yb, ¹⁷⁵Yb, ⁶⁵Zn; positron emitting metals; and magnetic metal ions positron emitting metals; and magnetic metal ions.

An anti-MASP-2 antibody or antigen-binding fragment described herein can be attached to a solid support. Such solid supports include, but are not limited to, glass, cellulose, polyacrylamide, nylon, polystyrene, polyvinyl chloride, or polypropylene. In some embodiments, an immobilized anti-MASP-2 antibody or antigen-binding fragment is used in an immunoassay. In some embodiments, an immobilized anti-MASP-2 antibody or antigen-binding fragment is used in purification of the target antigen (e.g., human MASP-2).

### 5.4 Polynucleotides and Vectors

Also provided herein are polynucleotides that encode a polypeptide (e.g., an anti-MASP-2 antibody or antigen-binding fragment) described herein. The term “polynucleotide that encode a polypeptide” encompasses a polynucleotide which includes only coding sequences for the polypeptide as well as a polynucleotide which includes additional coding and/or non-coding sequences. The polynucleotides of the disclosure can be in the form of RNA or in the form of DNA. DNA can be cDNA, genomic DNA, or synthetic DNA, and can be double-stranded or single-stranded. Single stranded DNA can be the coding strand or non-coding (anti-sense) strand. The polynucleotides of the disclosure can be mRNA.

Expressly contemplated herein are polynucleotides encode any anti-MASP-2 antibody or antigen-binding fragment disclosed herein. For illustrative purposes, in some embodiments, the polynucleotides provided herein encode an anti-MASP-2 antibody or antigen-binding fragment comprising (1) as defined by Kabat, (a) a light chain variable region (VL) comprising VL CDR1, VL CDR2, VL CDR3 having the amino acid sequences of SEQ ID NOs: 7, 9, and 11, respectively; or a variant thereof having up to about 5 amino acid substitutions, additions, and/or deletions in the VL CDRs; and/or (b) a heavy chain variable region (VH) comprising VH CDR1, VH CDR2, VH CDR3 having the amino acid sequences of SEQ ID NOs: 12, 16, and 18, respectively; or a variant thereof having up to about 5 amino acid substitutions, additions, and/or deletions in the VH CDRs; or (2) as defined by IMGT, (a) a VL comprising VL CDR1, VL CDR2, VL CDR3 having the amino acid sequences of SEQ ID NOs: 8, 10, and 11, respectively; or a variant thereof having up to about 5 amino acid substitutions, additions, and/or deletions in the VL CDRs; and/or (b) a VH comprising VH CDR1, VH CDR2, VH CDR3 having the amino acid sequences of SEQ ID NOs: 13, 17, and 19, respectively; or a variant thereof having up to about 5 amino acid substitutions, additions, and/or deletions in the VH CDRs.

In some embodiments, the polynucleotides provided herein encode an anti-MASP-2 antibody or antigen-binding fragment comprising (a) a VL having at least 85%, at least 90%, at least 95%, at least 98%, or 100% sequence identity to the amino acid sequence of SEQ ID NO: 23; and/or (b) a VH having at least 85%, at least 90%, at least 95%, at least 98%, or 100% sequence identity to the amino acid sequence of SEQ ID NO: 24. The polynucleotides can be in the form of DNA. The polynucleotides can be in the form of mRNA.

In some embodiments, the polynucleotides provided herein encode an anti-MASP-2 antibody or antigen-binding fragment disclosed herein comprising a VL and a VH, wherein the VL comprises VL CDR1, CDR2 and CDR3 and the VH comprises VH CDR1, CDR2 and CDR3, and wherein the VL CDR1, VL CDR2, VL CDR3, VH CDR1, VH CDR2 and VH CDR3 have the amino acid sequences of (1) SEQ ID NOs: 7, 9, 11, 12, 16 and 18, respectively; (2) SEQ ID NOs: 8, 10, 11, 13, 17, and 19, respectively; (3) SEQ ID NOs: 8, 10, 11, 14, 17, and 19, respectively; (4) SEQ ID NOs: 8, 10, 11, 15, 17, and 19, respectively; (5) SEQ ID NOs: 8, 10, 11, 13, 17, and 20, respectively; (6) SEQ ID NOs: 8, 10, 11, 14, 17, and 20, respectively; (7) SEQ ID NOs: 8, 10, 11, 15, 17, and 20, respectively; or a variant thereof having up to about 5 amino acid substitutions, additions, and/or deletions in the CDRs. The polynucleotides can be in the form of DNA. The polynucleotides can be in the form of mRNA.

In some embodiments, the polynucleotides provided herein encode an anti-MASP-2 antibody or antigen-binding fragment disclosed herein comprising a VL and a VH, wherein the VL and VH have the amino acid sequences of (1) SEQ ID NOs: 21 and 22, respectively; (2) SEQ ID NOs: 23 and 24, respectively; (3) SEQ ID NOs: 23 and 25, respectively; (4) SEQ ID NOs: 23 and 26, respectively; (5) SEQ ID NOs: 23 and 27, respectively; (6) SEQ ID NOs: 23 and 28, respectively; (7) SEQ ID NOs: 23 and 29, respectively; (8) SEQ ID NOs: 23 and 30, respectively; (9) SEQ ID NOs: 23 and 31, respectively; or (10) SEQ ID NOs: 23 and 32, respectively. The polynucleotides can be in the form of DNA. The polynucleotides can be in the form of mRNA.

In some embodiments, the VL and VH are connected by a linker. The linker can be a flexible linker or a rigid linker. In some embodiments, the linker has the amino acid sequence of (GGGGS)n, n=1, 2, 3, 4, or 5 (SEQ ID NO: 77). In some embodiments, the linker has the amino acid sequence of (EAAAK)n, n=1, 2, 3, 4, or 5 (SEQ ID NO: 78). In some embodiments, the linker has the amino acid sequence of (PA)nP, n=1, 2, 3, 4, or 5 (SEQ ID NO: 79).

The present disclosure also provides variants of the polynucleotides described herein, wherein the variants encode, for example, fragments, analogs, and/or derivatives of an anti-MASP-2 antibody or antigen-binding fragment disclosed herein. In some embodiments, the present disclosure provides a polynucleotide having a nucleotide sequence at least about 80% identical, at least about 85% identical, at least about 90% identical, at least about 95% identical, at least about 96% identical, at least about 97% identical, at least about 98% identical, or at least about 99% identical to a polynucleotide sequence encoding an anti-MASP-2 antibody or antigen-binding fragment described herein. In some embodiments, the present disclosure provides a polynucleotide having a nucleotide sequence at least about 80% identical, at least about 85% identical, at least about 90% identical, at least about 95% identical, at least about 96% identical, at least about 97% identical, at least about 98% identical, or at least about 99% identical to a polynucleotide sequence encoding an anti-MASP-2 antibody or antigen-binding fragment described herein.

As used herein, the phrase “a polynucleotide having a nucleotide sequence at least about 95% identical to a polynucleotide sequence” means that the nucleotide sequence of the polynucleotide is identical to a reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (e.g., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, a polynucleotide comprises the coding sequence for a polypeptide (e.g., an antibody) fused in the same reading frame to a polynucleotide which aids in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In some embodiments, a polynucleotide comprises the coding sequence for a polypeptide (e.g., an antibody) fused in the same reading frame to a marker or tag sequence. For example, in some embodiments, a marker sequence is a hexa-histidine tag (HIS-tag) that allows for efficient purification of the polypeptide fused to the marker. In some embodiments, a marker sequence is a hemagglutinin (HA) tag derived from the influenza hemagglutinin protein when a mammalian host (e.g., COS-7 cells) is used. In some embodiments, the marker sequence is a FLAG™ tag. In some embodiments, a marker can be used in conjunction with other markers or tags.

In some embodiments, a polynucleotide is isolated. In some embodiments, a polynucleotide is substantially pure.

Vectors and cells comprising the polynucleotides described herein are also provided. In some embodiments, provided herein are vectors comprising a polynucleotide provided herein. The vectors can be expression vectors. In some embodiments, vectors provided herein comprise a polynucleotide encoding an anti-MASP-2 antibody or antigen-binding fragment described herein. In some embodiments, vectors provided herein comprise a polynucleotide encoding a polypeptide that is part of an anti-MASP-2 antibody or antigen-binding fragment described herein.

In some embodiments, provided herein are recombinant expression vectors, which can be used to amplify and express a polynucleotide encoding an anti-MASP-2 antibody or antigen-binding fragment described herein. For example, a recombinant expression vector can be a replicable DNA construct that includes synthetic or cDNA-derived DNA fragments encoding a polypeptide chain of an anti-MASP-2 antibody, operatively linked to suitable transcriptional and/or translational regulatory elements derived from mammalian, microbial, viral or insect genes. In some embodiments, a viral vector is used. DNA regions are “operatively linked” when they are functionally related to each other. For example, a promoter is operatively linked to a coding sequence if it controls the transcription of the sequence; or a ribosome binding site is operatively linked to a coding sequence if it is positioned so as to permit translation. In some embodiments, structural elements intended for use in certain expression systems include a leader sequence enabling extracellular secretion of translated protein by a host cell. In some embodiments, in situations where recombinant protein is expressed without a leader or transport sequence, a polypeptide can include an N-terminal methionine residue.

A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

In some embodiments, an anti-MASP-2 antibody or antigen-binding fragment described herein is expressed from one or more vectors. Suitable host cells for expression include prokaryotes, yeast cells, insect cells, or higher eukaryotic cells under the control of appropriate promoters. Appropriate cloning and expression vectors for use with bacterial, fungal, yeast, and mammalian cellular hosts, as well as methods of protein production, including antibody production are well-known in the art.

Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

The present disclosure also provides host cells comprising the polypeptides described herein, polynucleotides encoding polypeptides described herein, or vectors comprising such polynucleotides. In some embodiments, provided herein are host cells comprising a vector comprising a polynucleotide disclosed herein. In some embodiments, host cells provided herein comprise a vector comprising a polynucleotide encoding an anti-MASP-2 antibody or antigen-binding fragment described herein. In some embodiments, host cells provided herein comprise a vector comprising a polynucleotide encoding a polypeptide that is part of an anti-MASP-2 antibody or antigen-binding fragment described herein. In some embodiments, host cells provided herein comprise a polynucleotide encoding an anti-MASP-2 antibody or antigen-binding fragment described herein. In some embodiments, the cells produce the anti-MASP-2 antibodies or antigen-binding fragments described herein.

### 5.5 Methods of Production

Polynucleotides provided herein can be prepared, manipulated, and/or expressed using any of the well-established techniques known and available in the art. Many vectors can be used. Examples of vectors are plasmid, autonomously replicating sequences, and transposable elements. Exemplary transposon systems such as Sleeping Beauty and PiggyBac can be used, which can be stably integrated into the genome (e.g., Ivics et al., Cell, 91 (4): 501-510 (1997); Cadiñanos et al., (2007) Nucleic Acids Research. 35 (12): e87). Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells.

In some embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In some embodiments, the lymphotrophic herpes virus may be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. Typically, the host cell comprises the viral replication transactivator protein that activates the replication.

“Expression control sequences,” “control elements,” or “regulatory sequences” present in an expression vector are those non-translated regions of the vector-origin of replication, selection cassettes, promoters, enhancers, translation initiation signals (Shine Dalgarno sequence or Kozak sequence) introns, a polyadenylation sequence, 5′ and 3′ untranslated regions-which interact with host cellular proteins to carry out transcription and translation. Such elements can vary in their strength and specificity. Depending on the vector system and host utilized, any number of suitable transcription and translation elements, including ubiquitous promoters and inducible promoters can be used.

Illustrative ubiquitous expression control sequences that can be used in present disclosure include, but are not limited to, a cytomegalovirus (CMV) immediate early promoter, a viral simian virus 40 (SV40) promoter (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, and a β-actin promoter.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc. The anti-MASP-2 antibodies or antigen-binding fragments described herein can be produced by any method known in the art, including chemical synthesis and recombinant expression techniques. The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) MOLECULAR CLONING: A LABORATORY MANUAL, Cold Spring Harbor Laboratory Press; Sambrook et al. (1989), MOLECULAR CLONING: A LABORATORY MANUAL, Second Edition, Cold Spring Harbor Laboratory Press; Sambrook et al. (2001) MOLECULAR CLONING: A LABORATORY MANUAL, Cold Spring Harbor Laboratory Press, Cold Spring Harbor, NY; Ausubel et al., CURRENT PROTOCOLS IN MOLECULAR BIOLOGY, John Wiley & Sons (1987 and annual updates); CURRENT PROTOCOLS IN IMMUNOLOGY, John Wiley & Sons (1987 and annual updates) Gait (ed.) (1984) OLIGONUCLEOTIDE SYNTHESIS: A PRACTICAL APPROACH, IRL Press; Eckstein (ed.) (1991) OLIGONUCLEOTIDES AND ANALOGUES: A PRACTICAL APPROACH, IRL Press; Birren et al. (eds.) (1999) GENOME ANALYSIS: A LABORATORY MANUAL, Cold Spring Harbor Laboratory Press; Borrebaeck (ed.) (1995) ANTIBODY ENGINEERING, Second Edition, Oxford University Press; Lo (ed.) (2006) ANTIBODY ENGINEERING: METHODS AND PROTOCOLS (METHODS IN MOLECULAR BIOLOGY); Vol. 248, Humana Press, Inc; each of which is incorporated herein by reference in its entirety.

The polypeptides described herein (e.g., the anti-MASP-2 antibodies or antigen-binding fragments) can be produced and isolated using methods known in the art. Peptides can be synthesized, in whole or in part, using chemical methods (see, e.g., Caruthers (1980). Nucleic Acids Res. Symp. Ser. 215; Horn (1980); and Banga, A. K., THERAPEUTIC PEPTIDES AND PROTEINS, FORMULATION, PROCESSING AND DELIVERY SYSTEMS (1995) Technomic Publishing Co., Lancaster, PA). Peptide synthesis can be performed using various solid phase techniques (see, e.g., Roberge, Science 269:202 (1995); Merrifield, Methods. Enzymol. 289:3 (1997)) and automated synthesis may be achieved, e.g., using the ABI 431A Peptide Synthesizer (Perkin Elmer) in accordance with the manufacturer's instructions. Peptides can also be synthesized using combinatorial methodologies. Synthetic residues and polypeptides can be synthesized using a variety of procedures and methodologies known in the art (see, e.g., ORGANIC SYNTHESES COLLECTIVE VOLUMES, Gilman, et al. (Eds) John Wiley & Sons, Inc., NY). Modified peptides can be produced by chemical modification methods (see, for example, Belousov, Nucleic Acids Res. 25:3440 (1997); Frenkel, Free Radic. Biol. Med. 19:373 (1995); and Blommers, Biochemistry 33:7886 (1994)). Peptide sequence variations, derivatives, substitutions and modifications can also be made using methods such as oligonucleotide-mediated (site-directed) mutagenesis, alanine scanning, and PCR based mutagenesis. Site-directed mutagenesis (Carter et al., Nucl. Acids Res., 13:4331 (1986); Zoller et al., Nucl. Acids Res. 10:6487 (1987)), cassette mutagenesis (Wells et al., Gene 34:315 (1985)), restriction selection mutagenesis (Wells et al., Philos. Trans. R. Soc. London SerA 317:415 (1986)) and other techniques can be performed on cloned DNA to produce invention peptide sequences, variants, fusions and chimeras, and variations, derivatives, substitutions and modifications thereof.

The polypeptides described herein can be prepared using a wide variety of techniques known in the art including the use of hybridoma and recombinant technologies, or a combination thereof. In some embodiments, a recombinant expression vector is used to express a polynucleotide encoding a polypeptide described herein. For example, a recombinant expression vector can be a replicable DNA construct that includes synthetic or cDNA-derived DNA fragments encoding a polypeptide operatively linked to suitable transcriptional and/or translational regulatory elements derived from mammalian, microbial, viral or insect genes. In some embodiments, coding sequences of polypeptides disclosed herein can be ligated into such expression vectors for their expression in mammalian cells. In some embodiments, a viral vector is used. DNA regions are “operatively linked” when they are functionally related to each other. For example, a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In some embodiments, in situations where recombinant protein is expressed without a leader or transport sequence, a polypeptide can include an N-terminal methionine residue.

A wide variety of expression host/vector combinations can be employed. Suitable host cells for expression include prokaryotes, yeast cells, insect cells, or higher eukaryotic cells under the control of appropriate promoters. Appropriate cloning and expression vectors for use with bacterial, fungal, yeast, and mammalian cellular hosts, as well as methods of protein production, including antibody production are well-known in the art. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Useful expression vectors for eukaryotic hosts include, for example, vectors comprising expression control sequences from SV40, bovine papilloma virus, adenovirus, and cytomegalovirus. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Provided herein are anti-MASP-2 antibodies and antigen-binding fragments thereof that include but are not limited to monoclonal antibodies, polyclonal antibodies, synthetic antibodies, human antibodies, humanized antibodies, and antigen-binding fragments thereof.

Methods of antibody production are well-known in the art. See for example, in Harlow et al., Antibodies: A Laboratory Manual, (Cold Spring Harbor Laboratory Press, 2nd ed. 1988); Hammerling et al., in: MONOCLONAL ANTIBODIES AND T-CELL HYBRIDOMAS 563 681 (Elsevier, N.Y., 1981), each of which is incorporated herein by reference in its entirety. For in vivo use of antibodies in humans, it may be preferable to use human antibodies. Completely human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO98/16654, WO 96/34096, WO 96/33735, and WO 91/10741; each of which is incorporated herein by reference in its entirety. A human antibody can also be an antibody wherein the heavy and light chains are encoded by a nucleotide sequence derived from one or more sources of human DNA.

Human antibodies can also be produced using transgenic mice which are incapable of expressing functional endogenous immunoglobulins, but which can express human immunoglobulin genes. For example, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. For example, it has been described that the homozygous deletion of the antibody heavy chain joining region (JH) gene in chimeric and germ-line mutant mice results in complete inhibition of endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. For example, anti-MASP-2 antibodies directed against the human MASP-2 antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies, including, but not limited to, IgG1 (gamma 1) and IgG3. For an overview of this technology for producing human antibodies, see, Lonberg and Huszar (Int. Rev. Immunol., 13:65-93 (1995)). For a detailed discussion of this technology for producing human antibodies and human monoclonal antibodies and protocols for producing such antibodies, see, e.g., PCT Publication Nos. WO 98/24893, WO 96/34096, and WO 96/33735; and U.S. Pat. Nos. 5,413,923; 5,625,126; 5,633,425; 5,569,825; 5,661,016; 5,545,806; 5,814,318; and 5,939,598, each of which is incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above. For a specific discussion of transfer of a human germ-line immunoglobulin gene array in germ-line mutant mice that will result in the production of human antibodies upon antigen challenge see, e.g., Jakobovits et al., Proc. Natl. Acad. Sci. USA, 90:2551 (1993); Jakobovits et al., Nature, 362:255-258 (1993); Bruggermann et al., Year in Immunol., 7:33 (1993); and Duchosal et al., Nature, 355:258 (1992).

Human antibodies can also be derived from phage-display libraries (Hoogenboom et al., J. Mol. Biol., 227:381 (1991); Marks et al., J. Mol. Biol., 222:581-597 (1991); Vaughan et al., Nature Biotech., 14:309 (1996)). Phage display technology (McCafferty et al., Nature, 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson and Chiswell, Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of unimmunized mice. A repertoire of V genes from unimmunized human donors can be constructed and antibodies to a diverse array of antigens (including self-antigens) can be isolated essentially following the techniques described by Marks et al., J. Mol. Biol., 222:581-597 (1991), or Griffith et al., EMBO J., 12:725-734 (1993). See, also, U.S. Pat. Nos. 5,565,332 and 5,573,905, each of which is incorporated herein by reference in its entirety.

Human antibodies can also be generated by in vitro activated B cells (see, U.S. Pat. Nos. 5,567,610 and 5,229,275, each of which is incorporated herein by reference in its entirety). Human antibodies can also be generated in vitro using hybridoma techniques such as, but not limited to, that described by Roder et al. (Methods Enzymol., 121:140-167 (1986)).

Alternatively, in some embodiments, a non-human antibody is humanized, where specific sequences or regions of the antibody are modified to increase similarity to an antibody naturally produced in a human. In some embodiment, the antigen binding domain portion is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28 (4/5): 489-498; Studnicka et al., 1994, Protein Engineering, 7 (6): 805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13 (5): 353-60 (2000), Morea et al., Methods, 20 (3): 267-79 (2000), Baca et al., J. Biol. Chem., 272 (16): 10678-84 (1997), Roguska et al., Protein Eng., 9 (10): 895-904 (1996), Couto et al., Cancer Res., 55 (23 Supp): 5973s-5977s (1995), Couto et al., Cancer Res., 55 (8): 1717-22 (1995), Sandhu J S, Gene, 150 (2): 409-10 (1994), and Pedersen et al., J. Mol. Biol., 235 (3): 959-73 (1994), each of which is incorporated herein in its entirety by reference. Often, framework residues in the framework regions can be substituted with the corresponding residue from the CDR donor antibody to alter, preferably improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some FR residues are substituted by residues from analogous sites in rodent antibodies. Humanization of antibodies can also be achieved by veneering or resurfacing (EP 592,106; EP 519,596; Padlan, 1991, Molecular Immunology, 28 (4/5): 489-498; Studnicka et al., Protein Engineering, 7 (6): 805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993), the contents of which are incorporated herein by reference herein in their entirety).

Antibodies can be humanized with retention of high affinity for the target antigen and other favorable biological properties. For example, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

A humanized antibody retains a similar antigenic specificity as the original antibody, for example, the ability to bind human MASP-2 antigen. However, using certain methods of humanization, the affinity and/or specificity of binding of the antibody for a particular antigen can be increased using methods of “directed evolution,” as described by Wu et al., J. Mol. Biol., 294:151 (1999), the contents of which are incorporated herein by reference herein in their entirety.

### 5.6 Pharmaceutical Compositions

Provided herein are also pharmaceutical compositions comprising the anti-MASP-2 antibodies or antigen-binding fragments disclosed herein. In some embodiments, the pharmaceutical composition comprises a therapeutically effective amount of the anti-MASP-2 antibodies or antigen-binding fragments disclosed herein and a pharmaceutically acceptable carrier. In some embodiments, the pharmaceutical compositions are useful in inhibiting MASP-2-dependent complement activation. In some embodiments, the pharmaceutical compositions are useful in treating a disease or disorder associated with MASP-2-dependent complement activation.

In some embodiments, the pharmaceutical compositions provided herein comprise anti-MASP-2 antibodies or antigen-binding fragments provided herein. The anti-MASP-2 antibodies or antigen-binding fragments can be present at various concentrations. In some embodiments, the pharmaceutical compositions provided herein comprise soluble anti-MASP-2 antibodies or antigen-binding fragments provided herein at 1-1000 mg/mL. In some embodiments, the pharmaceutical compositions comprise soluble anti-MASP-2 antibodies or antigen-binding fragments provided herein at 10-500 mg/mL, 10-400 mg/mL, 10-300 mg/mL, 10-200 mg/mL, 10-100 mg/mL, 20-100 mg/mL, or 50-100 mg/mL. In some embodiments, the pharmaceutical compositions provided herein comprise anti-MASP-2 antibodies or antigen-binding fragments provided herein at about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 120 mg/mL, about 150 mg/mL, about 180 mg/mL, about 200 mg/mL, about 300 mg/mL, about 500 mg/mL, about 800 mg/mL, or about 1000 mg/mL. Dosages can be readily adjusted by those skilled in the art; for example, a decrease in purity may require an increase in dosage.

Provided herein are also kits for preparation of pharmaceutical compositions having the anti-MASP-2 antibodies or antigen-binding fragments disclosed herein. In some embodiments, the kit comprises the anti-MASP-2 antibodies or antigen-binding fragments disclosed herein and a pharmaceutically acceptable carrier in one or more containers. In another embodiment, the kits can comprise anti-MASP-2 antibodies or antigen-binding fragments disclosed herein for administration to a subject. In specific embodiments, the kits comprise instructions regarding the preparation and/or administration of the anti-MASP-2 antibodies or antigen-binding fragments.

In some embodiments, provided herein is a pharmaceutical composition comprising anti-MASP-2 antibodies or antigen-binding fragments or cells provided herein wherein the composition is suitable for local administration.

Pharmaceutically acceptable carriers that can be used in compositions provided herein include any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active ingredient (i.e., anti-MASP-2 antibodies or antigen-binding fragments) can be coated in a material to protect the active ingredient from the action of acids and other natural conditions that can inactivate the active ingredient.

Provided herein are also pharmaceutical compositions or formulations that improve the stability of the anti-MASP-2 antibodies or antigen-binding fragments to allow for their long-term storage. In some embodiments, the pharmaceutical composition or formulation disclosed herein comprises: (a) anti-MASP-2 antibodies or antigen-binding fragments disclosed herein; (b) a buffering agent; (c) a stabilizing agent; (d) a salt; (e) a bulking agent; and/or (f) a surfactant. In some embodiments, the pharmaceutical composition or formulation is stable for at least 1 month, at least 2 months, at least 3 months, at least 6 months, at least 1 year, at least 2 years, at least 3 years, at least 5 years or more. In some embodiments, the pharmaceutical composition or formulation is stable when stored at 4° C., 25° C., or 40° C.

Buffering agents useful in the pharmaceutical compositions or formulations disclosed herein can be a weak acid or base used to maintain the acidity (pH) of a solution near a chosen value after the addition of another acid or base. Suitable buffering agents can maximize the stability of the pharmaceutical formulations by maintaining pH control of the formulation. Suitable buffering agents can also ensure physiological compatibility or optimize solubility. Rheology, viscosity and other properties can also depend on the pH of the formulation. Common buffering agents include, but are not limited to, histidine, citrate, succinate, acetate and phosphate. In some embodiments, a buffering agent comprises histidine (e.g., L-histidine) with isotonicity agents and potentially pH adjustment with an acid or a base known in the art. In certain embodiments, the buffering agent is L-histidine. In certain embodiments, the pH of the formulation is maintained between about 2 and about 10, or between about 4 and about 8.

Stabilizing agents are added to a pharmaceutical product to stabilize that product. Such agents can stabilize proteins in different ways. Common stabilizing agents include, but are not limited to, amino acids such as glycine, alanine, lysine, arginine, or threonine, carbohydrates such as glucose, sucrose, trehalose, rafftnose, or maltose, polyols such as glycerol, mannitol, sorbitol, cyclodextrins or destrans of any kind and molecular weight, or PEG. In some embodiments, the stabilizing agent is chosen to maximize the stability of FIX polypeptide in lyophilized preparations. In certain embodiments, the stabilizing agent is sucrose and/or arginine.

Bulking agents can be added to a pharmaceutical composition or formulation to add volume and mass to the product, thereby facilitating precise metering and handling thereof. Common bulking agents include, but are not limited to, lactose, sucrose, glucose, mannitol, sorbitol, calcium carbonate, or magnesium stearate.

Surfactants are amphipathic substances with lyophilic and lyophobic groups. A surfactant can be anionic, cationic, zwitterionic, or nonionic. Examples of nonionic surfactants include, but are not limited to, alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, or dodecyl dimethylamine oxide. In some embodiments, the surfactant is polysorbate 20 or polysorbate 80.

The pharmaceutical compositions disclosed herein can further comprise one or more of a buffer system, a preservative, a tonicity agent, a chelating agent, a stabilizer and/or a surfactant, as well as various combinations thereof. The use of preservatives, isotonic agents, chelating agents, stabilizers and surfactants in pharmaceutical compositions is well-known to the skilled person. Reference may be made to Remington: The Science and Practice of Pharmacy, 19^{th} edition, 1995.

In some embodiments, the pharmaceutical composition is an aqueous formulation. Such a formulation is typically a solution or a suspension, but can also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In some embodiments, the pharmaceutical compositions disclosed herein are freeze-dried, to which the physician or the patient adds solvents and/or diluents prior to use.

Pharmaceutical compositions disclosed herein can also include a pharmaceutically acceptable antioxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that can be employed in the pharmaceutical compositions or formulations described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. A pharmaceutical composition or formulation can comprise a preservative or can be devoid of a preservative. Supplementary active compounds can be incorporated into the compositions.

Pharmaceutical compositions or formulations typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, the compositions can include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

The amount of active ingredient which can be combined with a carrier material in the pharmaceutical compositions or formulations disclosed herein can vary. In some embodiments, the amount of active ingredient which can be combined with a carrier material is the amount that produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

The pharmaceutical compositions disclosed herein can be prepared with carriers that protect the active ingredient against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and poly lactic acid. Many methods for the preparation of such formulations are patented or generally known to those skilled in the art. See. e.g., Sustained and Controlled Release Drug Delivery Systems, J. R. Robinson, ed., Marcel Dekker, Inc., New York, 1978.

In some embodiments, the anti-MASP-2 antibodies or antigen-binding fragments described herein can be formulated to ensure proper distribution in vivo. For example, the blood-brain barrier (BBB) excludes many highly hydrophilic compounds. To ensure that the activate ingredient described herein cross the BBB, they can be formulated, for example, in liposomes. For methods of manufacturing liposomes, see, e.g., U.S. Pat. Nos. 4,522,811; 5,374,548; and 5,399,331. The liposomes can comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al) mannosides (Umezawa et al, (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); pl20 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

Anti-MASP-2 antibodies or antigen-binding fragments described herein can be tested for binding to human MASP-2 by, for example, standard ELISA. Briefly, microtiter plates are coated with purified MASP-2, and then blocked with bovine serum albumin. Dilutions of antibody (e.g., dilutions of plasma from MASP-2-immunized mice) are added to each well and incubated. The plates are washed and incubated with secondary reagent (e.g., for human antibodies, a goat-anti-human IgG Fc-specific polyclonal reagent) conjugated to horseradish peroxidase (HRP). After washing, the plates can be developed and analyzed by a spectrophotometer. Sera from immunized mice can then be further screened by flow cytometry for binding to a cell line expressing human MASP-2, but not to a control cell line that does not express MASP-2. Briefly, the binding of anti-MASP-2 antibodies can be assessed by incubating MASP-2 expressing CHO cells with the anti-MASP-2 antibody. The cells can be washed, and binding can be detected with an anti-human IgG Ab. Flow cytometric analyses can be performed using a FACS can flow cytometry (Becton Dickinson, San Jose, CA). Mice which develop the highest titers can be used for fusions.

An ELISA assay as described above can be used to screen for antibodies and, thus, hybridomas that produce antibodies that show positive reactivity with the MASP-2 immunogen. Hybridomas that produce antibodies that bind with high affinity to MASP-2 can then be subcloned and further characterized. One clone from each hybridoma, which retains the reactivity of the parent cells (by ELISA), can then be chosen for making a cell bank, and for antibody purification.

To purify anti-MASP-2 antibodies, selected hybridomas can be grown for monoclonal antibody purification. Supernatants can be filtered and concentrated before affinity chromatography. Eluted IgG can be checked by gel electrophoresis and high-performance liquid chromatography to ensure purity. The buffer solution can be exchanged, and the concentration can be determined. The monoclonal antibodies can be aliquoted and stored.

To determine if the selected anti-MASP-2 monoclonal antibodies bind to unique epitopes, each antibody can be biotinylated using commercially available reagents (Pierce, Rockford, IL). Biotinylated MAb binding can be detected with a streptavidin labeled probe. Competition studies using unlabeled monoclonal antibodies and biotinylated monoclonal antibodies can be performed using MASP-2 coated-ELISA plates as described above.

To determine the isotype of purified antibodies, isotype ELISAs can be performed using reagents specific for antibodies of a particular isotype. For example, to determine the isotype of a human monoclonal antibody, wells of microtiter plates can be coated with 1 pg/mL of anti-human immunoglobulin overnight at 4° C. After blocking with 1% BSA, the plates are reacted with 1 fig/mL or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

To test the binding of monoclonal antibodies to live cells expressing MASP-2, flow cytometry can be used, as described in the Examples. Briefly, cell lines expressing membrane-bound MASP-2 (grown under standard growth conditions) are mixed with various concentrations of monoclonal antibodies in PBS containing 0.1% BSA at 4° C. for 1 hour. After washing, the cells are reacted with Fluorescein-labeled anti-IgG antibody under the same conditions as the primary antibody staining. The samples can be analyzed by FACScan instrument using light and side scatter properties to gate on single cells and binding of the labeled antibodies is determined. An alternative assay using fluorescence microscopy can be used (in addition to or instead of) the flow cytometry assay. Cells can be stained exactly as described above and examined by fluorescence microscopy. This method allows visualization of individual cells, but can have diminished sensitivity depending on the density of the antigen.

Anti-MASP-2 antibodies or antigen-binding fragments can be further tested for reactivity with the MASP-2 antigen by Western blotting. Briefly, cell extracts from cells expressing MASP-2 can be prepared and subjected to sodium dodecyl sulfate polyacrylamide gel electrophoresis. After electrophoresis, the separated antigens will be transferred to nitrocellulose membranes, blocked with 20% mouse serum, and probed with the monoclonal antibodies to be tested. IgG binding can be detected using anti-IgG alkaline phosphatase and developed with BCIP/NBT substrate tablets (Sigma Chem. Co., St. Louis, MO).

Methods for analyzing binding affinity, cross-reactivity, and binding kinetics of various anti-MASP-2 antibodies include standard assays known in the art, for example, biolayer interferometry (BLI) using, for example, Gator system (Probe Life) or the Octet-96 system (Sartorius AG), or BIACORE™ surface plasmon resonance (SPR) analysis using a BIACORE™ 2000 SPR instrument (Biacore AB, Uppsala, Sweden).

### 5.7 Methods and Uses

The antibodies or antigen-binding fragments, compositions and methods described herein have numerous in vitro and in vivo utilities involving, for example, reducing inflammation, such as by inhibiting (or antagonizing) MASP-2-dependent complement activation. The antibodies or antigen-binding fragments provided herein can also be used in detection of MASP-2. In some embodiments, the anti-MASP-2 antibodies or antigen-binding fragments described herein are humanized antibodies or antigen-binding fragments. For example, anti-MASP-2 antibodies or antigen-binding fragments described herein can be administered to cells in culture, in vitro or ex vivo, or to human subjects, e.g., in vivo, to selectively inhibits lectin pathway complement activation in a variety of diseases. Accordingly, provided herein are methods of modifying complement activation in a subject comprising administering to the subject an anti-MASP-2 antibody, or antigen binding portion thereof, described herein such that the complement activation in the subject is modified.

Also encompassed are methods for detecting the presence of human MASP-2 antigen in a sample, or measuring the amount of human MASP-2 antigen, comprising contacting the sample, and a control sample, with a monoclonal antibody, e.g., a humanized monoclonal antibody, or an antigen binding portion thereof, which specifically binds to human MASP-2, under conditions that allow for formation of a complex between the antibody or antigen-binding fragment and human MASP-2. The formation of a complex is then detected, wherein a difference complex formation between the sample compared to the control sample is indicative the presence of human MASP-2 antigen in the sample. Moreover, the anti-MASP-2 antibodies or antigen-binding fragments described herein can be used to purify human MASP-2 via immunoaffinity purification.

The present disclosure also provides methods of uses of the anti-MASP-2 antibodies or antigen-binding fragments, polynucleotides encoding such anti-MASP-2 antibodies or antigen-binding fragments, vectors comprising such polynucleotides, or pharmaceutical compositions having such antibodies or antigen-binding fragments disclosed herein in inhibiting MASP-2-dependent complement activation, or treating a disease or disorder associated with MASP-2-dependent complement activation.

In some embodiments, provided herein are methods of treating a disease or disorder associated with MASP-2-dependent complement activation in a subject in need thereof, comprising administering to the subject a therapeutically effective amount of the anti-MASP-2 antibodies or antigen-binding fragments disclosed herein. In some embodiments, provided herein are uses of the anti-MASP-2 antibodies or antigen-binding fragments disclosed herein in the treatment of a disease or disorder associated with MASP-2-dependent complement activation. In some embodiments, provided herein are uses of the anti-MASP-2 antibodies or antigen-binding fragments provided herein for the preparation of a medicament for the treatment of a disease or disorder associated with MASP-2-dependent complement activation.

In some embodiments, provided herein are methods of treating a disease or disorder associated with MASP-2-dependent complement activation in a subject in need thereof, comprising administering to the subject a therapeutically effective amount of the pharmaceutical composition disclosed herein. In some embodiments, provided herein are uses of the pharmaceutical composition disclosed herein in treatment of a disease or disorder associated with MASP-2-dependent complement activation. In some embodiments, provided herein are uses of the pharmaceutical composition provided herein for the preparation of a medicament for the treatment of a disease or disorder associated with MASP-2-dependent complement activation.

As known in the art, dysregulated, unwanted or excessive activation of the complement system and deposition of immune complexes cause inflammation and damages in various tissues, which have been implicated in the pathogenesis of a wide variety of diseases. In some embodiments, the disease or disorder associated with MASP-2-dependent complement activation that can be treated with the anti-MASP-2 antibodies or antigen-binding fragments, or pharmaceutical compositions provided herein is a renal disease or disorder, a vascular disease or disorder, a skin disease or disorder, an ophthalmologic disease or disorder, a nervous system disease or disorder, a blood disease or disorder, a musculoskeletal disease or disorder, a urogenital disease or disorder, a metabolic disease or disorder, an endocrine disease or disorder, a gastrointestinal disease or disorder, or a pulmonary disease or disorder.

In some embodiments, the disease or disorder associated with MASP-2-dependent complement activation that can be treated with the anti-MASP-2 antibodies or antigen-binding fragments, or pharmaceutical compositions provided herein is a renal condition. The renal condition can be IgA nephropathy (i.e., Berger's disease), including severe IgAN and IgAVN (IgA-vasculitis associated nephritis), lupus nephritis (LN), membranous nephropathy (MN), nephrotic syndrome, glomerular diseases: glomerulonephritis, membranous glomerulonephritis, C3 glomerulopathy (C3G), IgM nephropathy, chronic kidney disease, chronic renal failure. In some embodiments, provided herein are methods of treating IgA nephropathy in a subject in need thereof, comprising administering to the subject a therapeutically effective amount of the anti-MASP-2 antibodies or antigen-binding fragments provided herein. In some embodiments, provided herein are methods of treating severe IgAN using the anti-MASP-2 antibodies or antigen-binding fragments provided herein. In some embodiments, methods provided therein treat IgAVN using the anti-MASP-2 antibodies or antigen-binding fragments provided herein. In some embodiments, provided herein are methods of treating lupus nephritis (LN) in a subject in need thereof, comprising administering to the subject a therapeutically effective amount of the anti-MASP-2 antibodies or antigen-binding fragments provided herein. In some embodiments, provided herein are methods of treating membranous nephropathy (MN) in a subject in need thereof, comprising administering to the subject a therapeutically effective amount of the anti-MASP-2 antibodies or antigen-binding fragments provided herein. In some embodiments, provided herein are methods of treating C3 glomerulopathy (C3G) in a subject in need thereof, comprising administering to the subject a therapeutically effective amount of the anti-MASP-2 antibodies or antigen-binding fragments provided herein. In some embodiments, provided herein are methods of treating a disease or condition associated with proteinuria in a subject in need thereof, comprising administering to the subject a therapeutically effective amount of the anti-MASP-2 antibodies or antigen-binding fragments provided herein. In some embodiments, provided herein are methods of treating a disease or condition caused or exacerbated by fibrosis and/or inflammation in a subject in need thereof, comprising administering to the subject a therapeutically effective amount of the anti-MASP-2 antibodies or antigen-binding fragments provided herein.

In some embodiments, the disease or disorder associated with MASP-2-dependent complement activation that can be treated with the anti-MASP-2 antibodies or antigen-binding fragments, or pharmaceutical compositions provided herein is a vascular condition. The vascular condition can be, e.g., a thrombotic disease or disorder, a thrombotic microangiopathy (TMA), paroxysmal nocturnal hemoglobinuria (PNH), vasculitis, or an acquired hypercoagulable state, e.g., presence of a catheter in a central vein. In some embodiments, provided herein are methods of treating TMA in a subject in need thereof, comprising administering to the subject a therapeutically effective amount of the anti-MASP-2 antibodies or antigen-binding fragments provided herein. In some embodiments, the disease or disorder is TMA associated with hematopoietic stem cell transplantation (HSCT-TMA). In some embodiments, the disease or disorder is atypical hemolytic uremic syndrome (aHUS). In some embodiments, the disease or disorder is Thrombotic thrombocytopenia purpura (TTP). In some embodiments, the disease or disorder is TMA secondary to cancer. In some embodiments, the disease or disorder is a TMA secondary to chemotherapy. In some embodiments, the disease or disorder is TMA secondary to transplantation. In some embodiments, provided herein are methods of treating aHUS in a subject in need thereof, comprising administering to the subject a therapeutically effective amount of the anti-MASP-2 antibodies or antigen-binding fragments provided herein. In some embodiments, provided herein are methods of treating TTP in a subject in need thereof, comprising administering to the subject a therapeutically effective amount of the anti-MASP-2 antibodies or antigen-binding fragments provided herein.

In some embodiments, the anti-MASP-2 antibodies or antigen-binding fragments, or pharmaceutical compositions provided herein can be used to treat a subject that has undergone, is undergoing, or will undergo an organ or tissue transplant procedure, preferably a transplant procedure selected from the group consisting of organ allotransplantation, organ xenotransplantation organ and tissue graft. In some embodiments, the anti-MASP-2 antibodies or antigen-binding fragments, or pharmaceutical compositions provided herein can be used to treat a graft-versus-host disease.

In some embodiments, the anti-MASP-2 antibodies or antigen-binding fragments, or pharmaceutical compositions provided herein can be used to treat COVID-19 complications.

Actual dosage levels of the active ingredients (i.e., the anti-MASP-2 antibodies or antigen-binding fragments) in the pharmaceutical compositions described herein can be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions described herein, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

The anti-MASP-2 antibodies or antigen-binding fragments can be administered as a sustained release formulation, in which case less frequent administration is required. Dosage and frequency vary depending on the half-life of the anti-MASP-2 antibodies or antigen-binding fragments in the patient. In therapeutic applications, a relatively high dosage at relatively short intervals is sometimes required until progression of the disease is reduced or terminated, and until the patient shows partial or complete amelioration of symptoms of disease.

The anti-MASP-2 antibodies or antigen-binding fragments or pharmaceutical compositions provided herein can be administered to a subject by any methods known in the art, including, but not limited to, pleural administration, intravenous administration, subcutaneous administration, intranodal administration, intramuscular administration, intradermal administration, intrathecal administration, intrapleural administration, intraperitoneal administration, intracranial administration, spinal or other parenteral routes of administration, for example by injection or infusion, or direct administration to the thymus. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. In some embodiments, subcutaneous administration is adopted. In some embodiments, intravenous administration is adopted. In some embodiments, oral administration is adopted. In one embodiment, the antibodies or antigen-binding fragments provided herein can be delivered locally. In another embodiment, the antibodies or antigen-binding fragments provided herein can be administered systemically.

In the methods disclosed herein, a therapeutically effective amount of the anti-MASP-2 antibodies or antigen-binding fragments or pharmaceutical compositions disclosed herein is administered to a subject that can benefit from reduction in MASP-2-depement complement activation. The subject can be someone who can benefit from selective inhibition of lectin pathway complement activation. The subject can have unwanted, unregulated, or excessive complement activation of the lectin pathway. The subject can be a mammal. In some embodiments, the subject is a human.

Anti-MASP-2 antibodies or antigen-binding fragments or pharmaceutical compositions provided herein can be administered with medical devices known in the art. For example, in some embodiments, a needleless hypodermic injection device can be used, such as the devices disclosed in U.S. Pat. Nos. 5,399,163; 5,383,851; 5,312,335; 5,064,413; 4,941,880; 4,790,824; or 4,596,556. Examples of well-known implants and modules for use described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In some embodiments, the anti-MASP-2 antibodies or antigen-binding fragments or pharmaceutical compositions provided herein can be administered with an additional therapy. In some embodiments, the additional therapy to be administered with an anti-MASP-2 antibodies or antigen-binding fragments provided herein can be an anti-inflammatory and/or analgesic agent, an anti-restenosis agent, or another complement inhibitor. Exemplary anti-inflammatory and/or analgesic agents include, e.g., serotonin receptor antagonists; serotonin receptor agonists; histamine receptor antagonists; bradykinin receptor antagonists; kallikrein inhibitors; tachykinin receptor antagonists, including neurokinin and neurokinin receptor subtype antagonists; calcitonin gene-related peptide (CGRP) receptor antagonists; interleukin receptor antagonists; inhibitors of enzymes active in the synthetic pathway for arachidonic acid metabolites, including phospholipase inhibitors, including PLA2isoform inhibitors and PLCγ isoform inhibitors, cyclooxygenase (COX) inhibitors (COX-I selective, COX-2 selective, or nonselective COX-I and -2 inhibitors), lipooxygenase inhibitors; prostanoid receptor antagonists including eicosanoid EP-1 and EP-4 receptor subtype antagonists and thromboxane receptor subtype antagonists; leukotriene receptor antagonists including leukotriene B4 receptor subtype antagonists and leukotriene D4 receptor subtype antagonists; opioid receptor agonists, including μ-opioid, δ-opioid, and κ-opioid receptor subtype agonists; purinoceptor agonists and antagonists including P2X receptor antagonists and P2Y receptor agonists; adenosine triphosphate (ATP)-sensitive potassium channel openers; MAP kinase inhibitors; nicotinic acetylcholine inhibitors; and alpha adrenergic receptor agonists. Exemplary complement inhibitors can be a C3 inhibitor (e.g., pegcetacoplan) or an anti-C5 antibody (e.g., eculizumab or ravulizumab).

The additional therapy can be administered prior to, concurrently with, or subsequent to administration of the anti-MASP-2 antibodies or antigen-binding fragments, cells, or pharmaceutical compositions described herein. Combined administration can include co-administration, either in a single pharmaceutical formulation or using separate formulations, or consecutive administration in either order but generally within a time period such that all active agents can exert their biological activities simultaneously. A person skilled in the art can readily determine appropriate regimens for administering a pharmaceutical composition described herein and an additional therapy in combination, including the timing and dosing of an additional agent to be used in a combination therapy, based on the needs of the subject being treated.

### 5.8 EXPERIMENTAL

The examples provided below are for purposes of illustration only, which are not intended to be limiting unless otherwise specified. Thus, the invention should in no way be construed as being limited to the following examples, but rather, should be construed to encompass any and all variations which become evident as a result of the teaching provided herein.

Briefly, results from the studies described below demonstrate that clone 3E10 and its humanized versions bind to the protease domain of human MASP-2 with high affinity, and are much more potent than the benchmark antibody narsoplimab (“MASP-2-BM”) in blocking completement activation.

### 5.8.1 Example 1: Immunization and Antibody Screening

Mice from various strains (C57BL/6, BALB/c, SJL and CD-1) were immunized with recombinant human MASP-2 protein (SEQ ID NO: 1, NCBI accession number NP_006601.2). Those which generated a strong titer response were selected for single B cell isolation. B cells from spleen and lymph nodes were isolated and enriched with microbeads. The B cells which recognized human MASP-2 were stained and isolated by FACS. The immunoglobin heavy chain and light chain gene sequences of these B cells were cloned and recombinantly expressed. These monoclonal antibodies then were taken into re-screening for binding to and blocking activity against human MASP-2. Clone 3E10 was selected for further studies.

### 5.8.2 Example 2: Blocking Activity Evaluation Using C4 Activation Assay

Methods: A C4 activation assay was used to evaluate the blocking activity of the chimeric antibodies. 100 μl 10 μg/mL Mannan (Sigma-M7504) was coated onto 96 well ELISA plate at 4° C. overnight. Antibodies were serially diluted with C4 activation buffer (0.1% BSA+20 mM Tris-HCl+2 mM CaCl₂+140 mM NaCl+ 1 mM MgCl₂+0.05% Tween20, pH7.4). 55 μl serially diluted antibodies and 55 μl C4 activation buffer containing 2% human serum (Quidel-A113) were mixed and incubated on ice for 45 min. The mannan coated plate was washed with washing buffer (10 mM Tris-HCl+140 mM NaCl +0.05% Tween20+2 mM CaCl₂), pH 7.2-7.4) and blocked with blocking buffer (0.1% BSA+10 mM Tris-HCl+140 mM NaCl, pH 7.4). 100 μl antibody-serum mixtures were added into the mannan coated plate and incubated at 37° C. for 1.5 h. After wash, 100 μl HRP linked anti-C4 antibody (Quidel-A211, 1:3000) were added into plate and incubated for 1 h. TMB was used for detection of the activated complement C4.

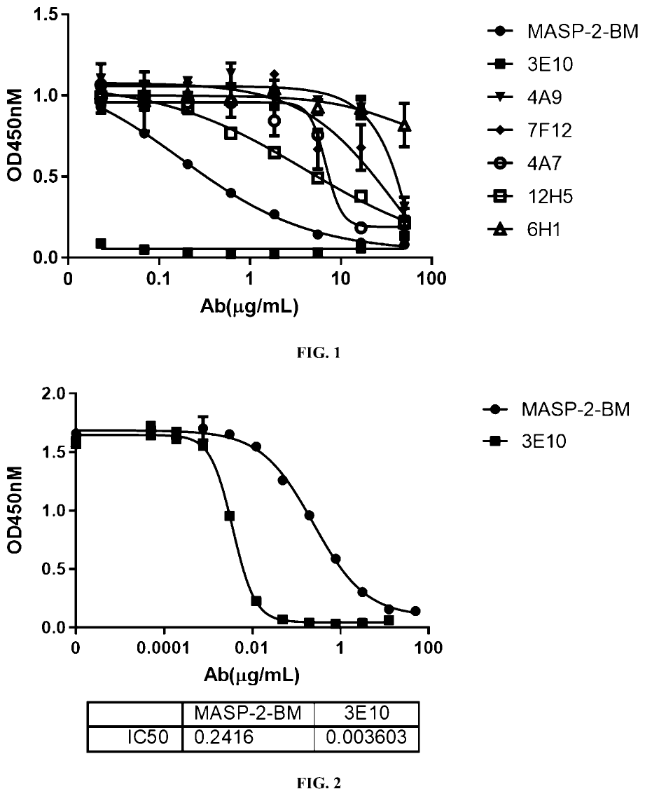

Results and conclusions: As shown in FIG. 1 and FIG. 2, chimeric antibodies blocked C4 activation to different extent. Clone 3E10 was most potent, with an IC₅₀ of 0.003603 μg/mL, whereas the IC₅₀ of MASP-2-BM is 0.2416 μg/mL.

### 5.8.3 Example 4: 3E10 Blocked C3 and MAC Activation

Methods: C3 and MAC activation assay: 100 μl 10 μg/mL Mannan (Sigma-M7504) was coated onto 96 well ELISA plate at 4° C. overnight. Antibodies were serially diluted with activation buffer (0.1% BSA+20 mM Tris-HCl+2 mM CaCl₂+140 mM NaCl+1 mM MgCl₂+0.05% Tween20, pH7.4). 55 μl serially diluted antibodies and 55 μl activation buffer containing 2% human serum (Quidel-A113) were mixed and incubated on ice for 45 min. The mannan coated plate was washed with washing buffer (10 mM Tris-HCl+140 mM NaCl+0.05% Tween 20+2 mM CaCl₂, pH 7.2-7.4) and blocked with blocking buffer (0.1% BSA+10 mM Tris-HCl+140 mM NaCl, pH 7.4). 100 μl antibody-serum mixtures were added into the mannan coated plate and incubated at 37° C. for 1.5 h. After wash, 100 μl HRP linked anti-C3 antibody (Quidel-A205, 1:3000) or HRP linked anti-MAC antibody (Quidel-A239, 1:3000) were added into plate and incubated for 1 h, respectively. TMB (3,3′,5,5″-tetramethylbenzidine) was used for detection of the activated complement C3 or MAC.

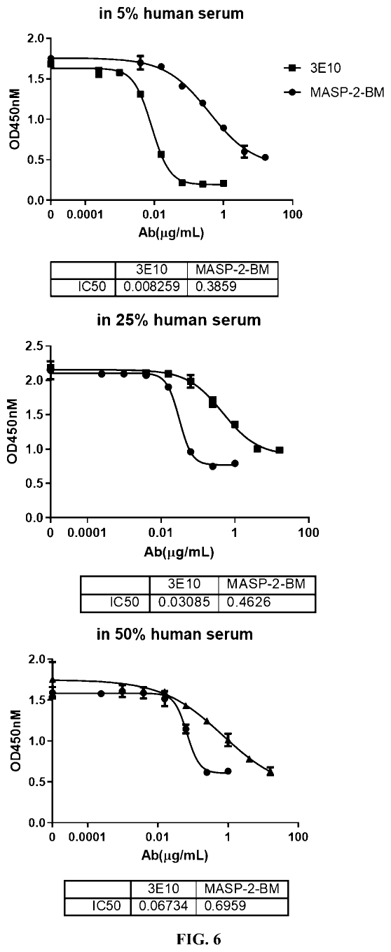

Result and conclusions: As shown in FIG. 4 and FIG. 5, chimeric 3E10 blocked C3 and MAC activation with an IC₅₀ of 0.004142 μg/mL and 0.003266 μg/mL respectively.

### 5.8.4 Example 3: Binding of 3E10 to MASP-2 Measured by BLI

Methods: Clone 3E10 was diluted with kinetics buffer (PBS pH 7.4, 0.1% BSA+0.01% Tween-20) to 100 nM. Recombinant MASP-2 protein was diluted with kinetics buffer to get a concentration gradient: 200 nM, 100 nM, 50 nM, 25 nM, and 0 nM (reference control). Antibodies were immobilized onto Protein A biosensor after balance. A baseline was detected for 60 seconds. Then, antibody-antigen association was detected for 120 seconds to obtain data for Kₒₙ, followed by detection of dissociation in kinetic buffer for 120 seconds to obtain data for K_{off}. The regeneration of biosensors was done in buffer 10 mM glycine, pH2.0. All the kinetics data were collected at 30° C. Data were acquired with Gator Bioanalysis System.

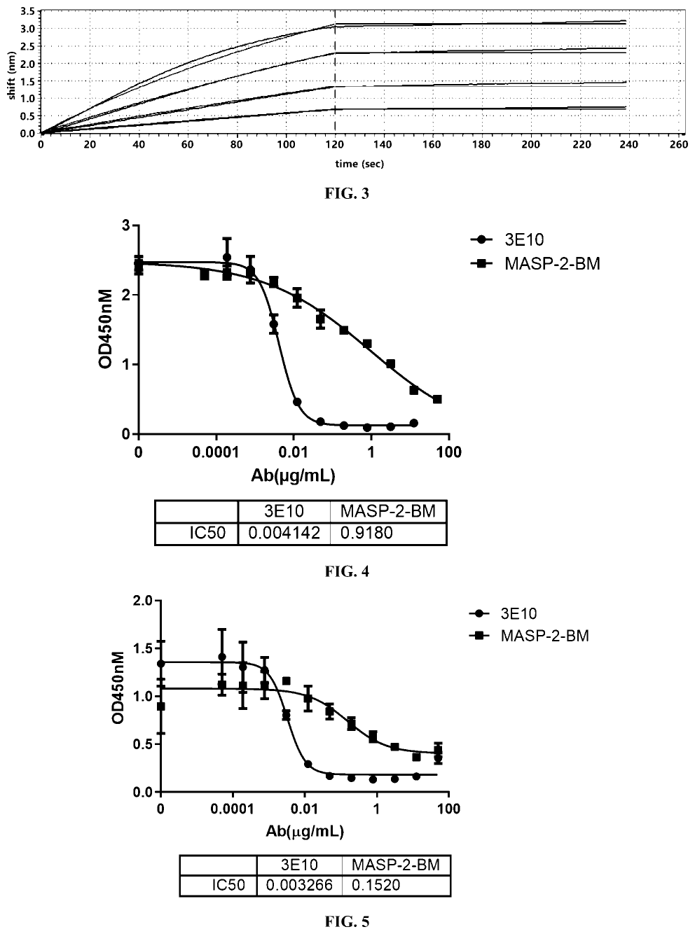

Results and conclusions: As shown in FIG. 3 and Table 4, chimeric 3E10 bound to human MASP-2 with a high affinity (K_{D} value<1E-10 M)

TABLE 4
Binding affinity of 3E10 to human MASP-2

| | Kon (1/Ms) | Koff (1/s) | K_{D} (M) |
|---|---|---|---|
| 3E10 | 2.96E+04 | <10E−7 | <1E−10 |

### 5.8.5 Example 5: 3E10 Blocked C4 Activation in Different Concentrations of Human Serum

Methods: C4 activation assay was used to evaluate the blocking activity of 3E10 in different concentrations of human serum. 100 μl 10 μg/mL Mannan (Sigma-M7504) was coated onto 96 well ELISA plate at 4° C. overnight. Antibodies were serially diluted with C4 activation buffer (0.1% BSA+20 mM Tris-HCl+2 mM CaCl₂+140 mM NaCl+1 mM MgCl₂+0.05% Tween20, pH7.4). 55 μl serially diluted antibodies and 55 μl C4 activation buffer containing different concentrations of human serum (Quidel-A113) were mixed and incubated on ice for 45 min. The final concentration of human serum was 5%, 25%, and 50% respectively. The mannan coated plate was washed with washing buffer (10 mM Tris-HCl+140 mM NaCl+0.05% Tween20+2 mM CaCl₂, pH 7.2-7.4) and blocked with blocking buffer (0.1% BSA+10 mM Tris-HCl+140 mM NaCl, pH 7.4). 100 μl antibody-serum mixtures were added into the mannan coated plate and incubated at 37° C. for 1.5 h. After wash, 100 μl HRP linked anti-C4 antibody (Quidel-A211, 1:3000) were added into plate and incubated for 1 h. TMB was used for detection of the activated complement C4.

Results and conclusions: As shown in FIG. 6, chimeric 3E10 blocked C4 activation in 5%, 10%, and 50% human serum with an IC₅₀ of 0.008259 μg/mL, 0.03085 μg/mL, and 0.06734 μg/mL respectively.

### 5.8.6 Example 6: Humanization of 3E10 and Binding Affinity Thereof to MASP-2 Measured by BLI

Methods: For the humanization of 3E10, IgBLAST from NCBI was used to choose the most appropriate human frameworks for grafting rodent CDRs. Variable regions with high amino acid sequence identity to the rodent variable regions (homology matching or best fit) were used. 3E10 was humanized by grafting the three CDRs from the light chain variable region into a human VL that was as homologous as possible to the mouse antibody VL. Similarly, their three CDRs from the heavy chain variable region were grafted into a human VH that was as homologous as possible to the mouse antibody. Kabat numbering was followed. Furthermore, in the framework region of the selected human variable regions, a few amino acid residues were changed into the amino acid residues that were present in the mouse variable regions (referred to as “back mutations”). Humanized sequences of 3E10 were listed in Table 3 above. Different humanization clones share one light chain sequence. The binding and activities of humanized 3E10 clones were evaluated by BLI and C4 activation assay. Experiment procedures of C4 activation assay were the same as described in Examples above.

The binding affinity of humanized 3E10 clones were measured by BLI with gator. Humanized 3E10 clones or MSAP-2-BM was immobilized on Protein A biosensor. The assay was performed at 30° C. and the kinetics buffer was 1×PBS (136 mM NaCl, 8 mM NaHPO₄, 2 mM KH₂PO₄, 2.6 mM KCl) with 0.1% BSA and 0.005% Tween-20, pH7.4. Diluted antibodies and human MASP-2 were added to the corresponding well. After loading human MASP-2, biosensor detected the association and dissociation of antibodies. The regeneration of biosensors was in buffer 10 mM glycine, pH2.0.

Results and conclusions: As shown in Table 5, humanized 3E10 clones maintained high binding affinity to human MASP-2 comparable to chimeric 3E10. Also shown in FIG. 7, humanized 3E10demonstrated high C4 blocking activity comparable to chimeric 3E10.

TABLE 5
Binding affinity of humanized 3E10 (tested by BLI)

| Ab name | koff(1/s) | kon(1/Ms) | KD(M) |
|---|---|---|---|
| MASP-2-BM | 0.00108 | 71900 | 1.50E−08 |
| 3E10-mus | 0.00113 | 73500 | 1.54E−08 |
| 3E10-hu-0 | 102 | 38700 | 2.63E−08 |
| 3E10-hu-6 | 0.000985 | 64600 | 1.52E−08 |
| 3E10-hu-7-1 | 0.00114 | 65700 | 1.73E−08 |
| 3E10-hu-7-2 | 0.00107 | 72900 | 1.47E−08 |
| 3E10-hu-8 | 0.00107 | 68600 | 1.56E−08 |
| 3E10-hu-9 | 0.000952 | 74200 | 1.28E−08 |
| 3E10-hu-10 | 0.00111 | 72800 | 1.53E−08 |
| 3E10-hu-11 | 0.00107 | 77700 | 1.37E−08 |
| 3E10-hu-12 | 0.00103 | 72300 | 1.43E−08 |

### 5.8.7 Example 7: Binding of Humanized 3E10 to MASP-2 Measured by SPR

Methods: The binding affinity of humanized 3E10 were further measured by surface plasmon resonance (SPR) technology with Biacore 8K. Humanized 3E10 or MASP-2-BM was immobilized on CM-5 chip. The assay was performed at 25° C. and the running buffer was 1×HEPES (10 mM HEPES, 150 mM NaCl, 3 mM EDTA) with 0.005% Tween-20, pH7.4. Diluted antibodies were captured on the sensor chip through Fc capture method. Human MASP-2 was used as the analyte, followed by injecting running buffer as dissociation phase.

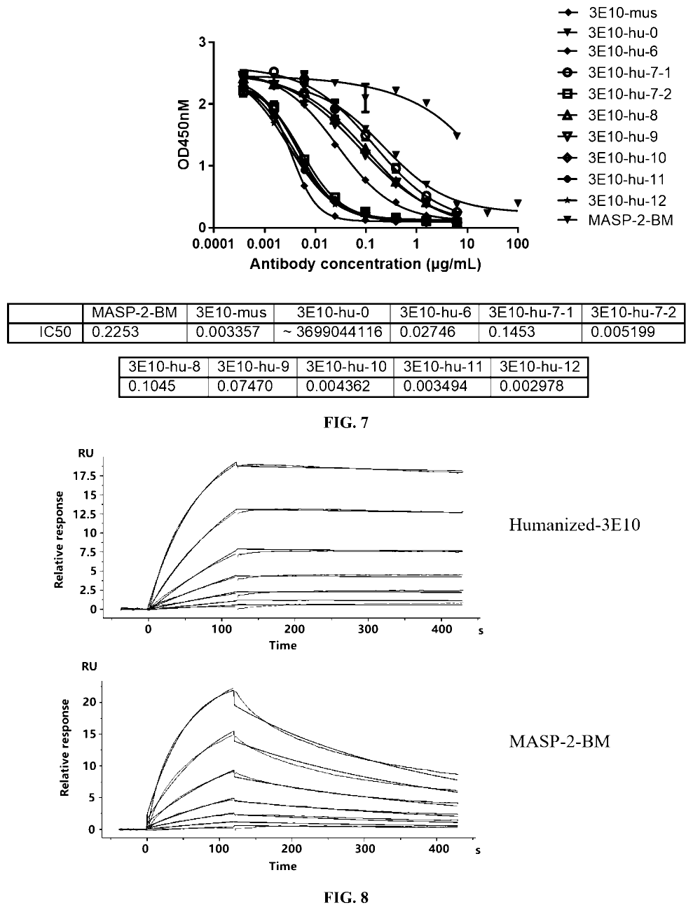

Results and conclusions: Representative results are provided in FIG. 8 and Table 6, confirming that humanized 3E10 bound to MASP-2 with a much lower K_{D} than that of the benchmark antibody.

TABLE 6
Binding affinity of antibodies to hMASP-2 (tested by SPR)

| Ligand | Analyte Conc. | kon (1/Ms) | koff (1/s) | K_{D} (M) | Rmax (RU) | Chi² (RU²) | Fit model |
|---|---|---|---|---|---|---|---|
| h3E10 | 0.195-12.5 nM | 1130000 | 0.000108 | 9.58E−11 | 23.1 | 0.03 | 1:1 binding |
| MASP-2-BM | 0.195-12.5 nM | 1990000 | 0.00386 | 1.94E−09 | 24.2 | 0.09 | 1:1 binding |

### 5.8.8 Example 8: Humanized 3E10 Blocked C4, C3 and MAC Activation

Methods: Human serum samples were collected from 10 healthy donors by a standard method without clot activators. C4, C3 and MAC assays were performed following the same procedure as described in Examples above for humanized 3E10 disclosed herein.

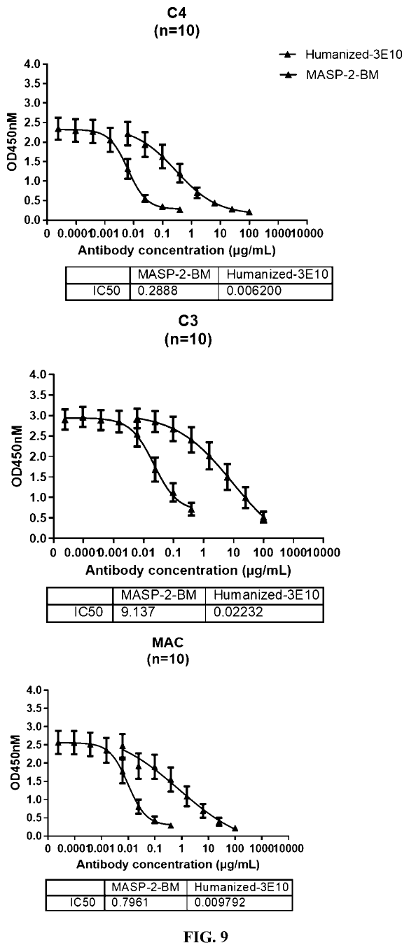

Results and conclusions: Representative results are provided in FIG. 9. As shown, humanized 3E10 strongly blocked C4, C3 and MAC activation in serum from healthy donors with an IC₅₀ value of 0.006200 μg/mL, 0.02232 μg/mL, and 0.009792 μg/mL, much lower than those of the benchmark antibody.

### 5.8.9 Example 9: Humanized 3E10 had No Effect on Classical or Alternative Pathway

Methods: For classical pathway activation, 3 μg/mL IgM antibody was coated onto the 96 well ELISA plate at 4° C. overnight. Antibodies were serially diluted with activation buffer (0.1% BSA+20 mM Tris-HCl+2 mM CaCl₂+140 mM NaCl+1 mM MgCl₂+0.05% Tween20, pH7.4) containing 40% human serum, mixed and incubated on ice for 45 min. 10 mM EDTA was used as a positive control. The IgM coated plate was washed with washing buffer (10 mM Tris-HCl+140 mM NaCl+0.05% Tween 20+2 mM CaCl₂, pH 7.2-7.4) and blocked with blocking buffer (0.1% BSA+10 mM Tris-HCl+140 mM NaCl, pH 7.4). 100 μl antibody-serum mixtures were added into the IgM coated plate and incubated at 37° C. for 1.5 h. After wash, 100 μl HRP linked anti-MAC antibody were added into plate and incubated for 1 h. TMB was used for detection of the formation of MAC.

For alternative pathway activation, 3 μg/mL LPS was diluted in PBS with 10 mM MgCl₂ and coated onto 96 well ELISA plate at 4° C. overnight. Antibodies were serially diluted with activation buffer (20 mM Tris-HCl+10 mM EGTA+5 mM MgCl₂, pH7.4) containing 10% human serum, mixed and incubated on ice for 45 min. 10 mM EDTA was used as a positive control. The LPS coated plate was washed with washing buffer (10 mM Tris-HCl+140 mM NaCl+0.05% Tween20+2 mM CaCl₂, pH 7.2-7.4). 100 μl antibody-serum mixtures were added into the LPS coated plate and incubated at 37° C. for 1.5 h. After wash, 100 μl HRP linked anti-MAC antibody were added into plate and incubated for 1 h. TMB was used for detection of the formation of MAC.

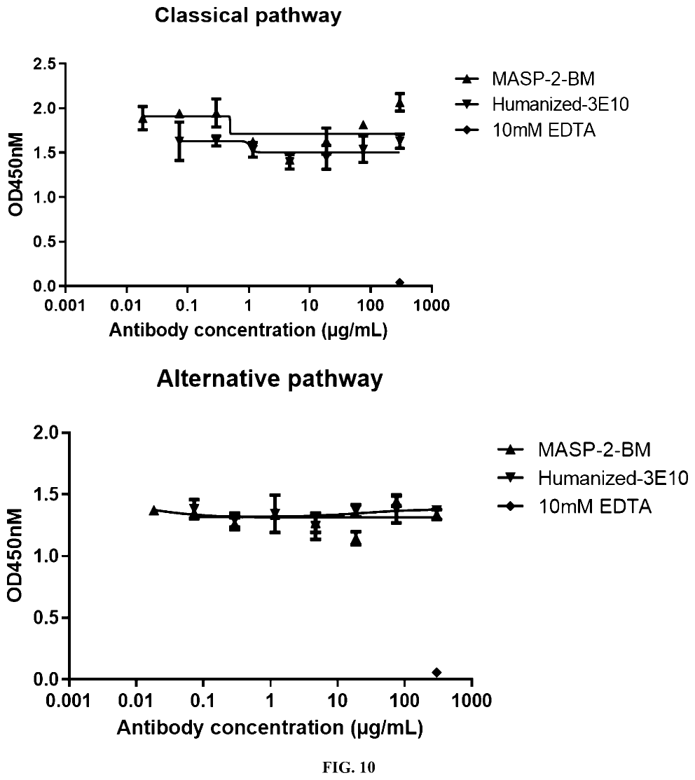

Results and conclusions: Representative results are provided in FIG. 10. As shown, humanized 3E10 had no effect on MAC deposition generated from classical pathway or alternative pathway activation.

### 5.8.10 Example 10: Epitope Binning of 3E10

Methods: MASP-2-his, 3E10 or MASP-2-BM was diluted with kinetics buffer (PBS pH 7.4, 0.1% BSA+0.01% Tween-20) to a concentration of 100 nM. MASP-2-his was immobilized on anti-his biosensor after balance. A baseline was detected for 60 seconds. The 1st antibody association was detected for 240 seconds and followed by balance. Then the 2nd antibody association was detected for 240 seconds. The regeneration of biosensors was done in buffer 10 mM glycine, pH2.0. All the kinetics data were collected at 30° C. Data were acquired with Gator Bioanalysis System.

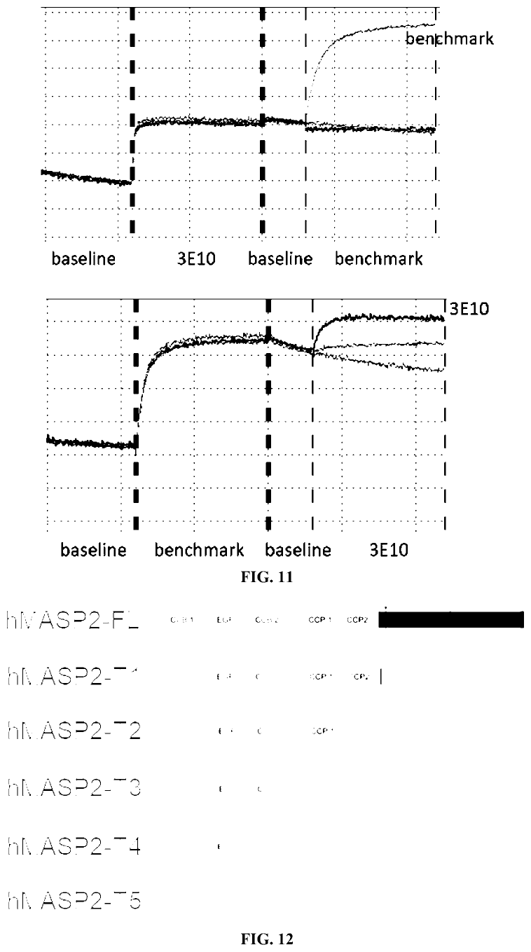

Results and conclusions: As shown in FIG. 11, the binding of 1st antibody did not influence the binding of 2nd antibody, indicating that 3E10 and MASP-2-BM bind to different epitopes on MASP-2.

### 5.8.11 Example 11: Epitope Binning of Humanized 3E10

Methods: As shown in FIG. 12, truncated human MASP-2 proteins were designed for epitope mapping. Sequences of these antigens were listed in Table 7. Genes encoding these antigens were synthesized and expressed in CHO expression system. The binding of the humanized 3E10 disclosed herein or the benchmark antibody to different truncated human MASP-2 proteins was tested by ELISA.

TABLE 7
Truncated human MASP-2

| Truncated | |
|---|---|
| MASP-2 | Sequences |
| MASP-2-T1 | TPLGPKWPEPVFGRLASPGFPGEYANDQERRWTLTAPPGYRLRLYFTHFDLELSHLCEYDFVKL |
| (aa 1-414) | SSGAKVLATLCGQESTDTERAPGKDTFYSLGSSLDITFRSDYSNEKPFTGFEAFYAAEDIDECQ |
| | VAPGEAPTCDHHCHNHLGGFYCSCRAGYVLHRNKRTCSALCSGQVFTQRSGELSSPEYPRPYPK |
| | LSSCTYSISLEEGFSVILDEVESFDVETHPETLCPYDELKIQTDREEHGPFCGKTLPHRIETKS |
| | NTVTITFVTDESGDHTGWKIHYTSTAQPCPYPMAPPNGHVSPVQAKYILKDSFSIFCETGYELL |
| | QGHLPLKSFTAVCQKDGSWDRPMPACSIVDCGPPDDLPSGRVEYITGPGVTTYKAVIQYSCEET |
| | FYTMKVNDGKYVCEADGFWTSSKGEKSLPV (SEQ ID NO: 2) |
| MASP-2-T2 | TPLGPKWPEPVFGRLASPGFPGEYANDQERRWTLTAPPGYRLRLYFTHFDLELSHLCEYDFVKL |
| (aa 1-345) | SSGAKVLATLCGQESTDTERAPGKDTFYSLGSSLDITFRSDYSNEKPFTGFEAFYAAEDIDECQ |
| | VAPGEAPTCDHHCHNHLGGFYCSCRAGYVLHRNKRTCSALCSGQVFTQRSGELSSPEYPRPYPK |
| | LSSCTYSISLEEGFSVILDFVESFDVETHPETLCPYDELKIQTDREEHGPFCGKTLPHRIETKS |
| | NTVTITFVTDESGDHTGWKIHYTSTAQPCPYPMAPPNGHVSPVQAKYILKDSFSIFCETGYELL |
| | QGHLPLKSFTAVCQKDGSWDRPMPA (SEQ ID NO: 3) |
| MASP-2-T3 | TPLGPKWPEPVFGRLASPGFPGEYANDQERRWTLTAPPGYRLRLYFTHFDLELSHLCEYDFVKL |
| (aa 1-279) | SSGAKVLATLCGQESTDTERAPGKDTFYSLGSSLDITFRSDYSNEKPFTGFEAFYAAEDIDECQ |
| | VAPGEAPTCDHHCHNHLGGFYCSCRAGYVLHRNKRTCSALCSGQVFTQRSGELSSPEYPRPYPK |
| | LSSCTYSISLEEGFSVILDFVESFDVETHPETLCPYDELKIQTDREEHGPFCGKTLPHRIETKS |
| | NTVTITFVTDESGDHTGWKIHYT (SEQ ID NO: 4) |
| MASP-2-T4 | TPLGPKWPEPVFGRLASPGFPGEYANDQERRWTLTAPPGYRLRLYFTHFDLELSHLCEYDFVKL |
| (aa 1-166) | SSGAKVLATLCGQESTDTERAPGKDTFYSLGSSLDITFRSDYSNEKPFTGFEAFYAAEDIDECQ |
| | VAPGEAPTCDHHCHNHLGGFYCSCRAGYVLHRNKRTCS (SEQ ID NO: 5) |
| MASP-2-T5 | TPLGPKWPEPVFGRLASPGFPGEYANDQERRWTLTAPPGYRLRLYFTHFDLELSHLCEYDFVKL |
| (aa 1-122) | SSGAKVLATLCGQESTDTERAPGKDTFYSLGSSLDITFRSDYSNEKPFTGFEAFYAAE (SEQ |
| | ID NO: 6) |

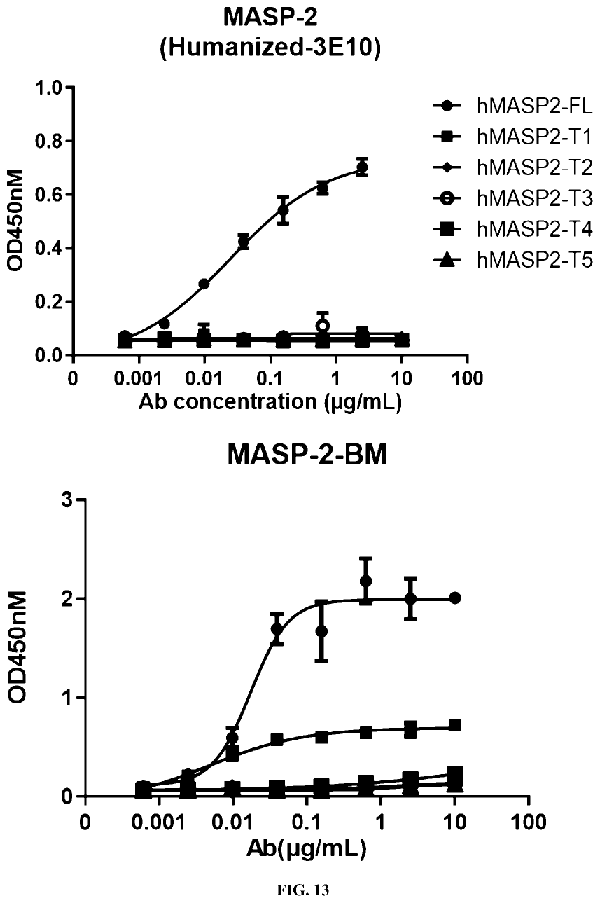

Results and conclusions: Representative results are provided in FIG. 13. As shown, humanized 3E10 completely lost its binding to the truncated human MASP-2 that lacked the protease domain, demonstrating that the protease domain of human MASP-2 was required for the humanized 3E10 to bind. By contrast, the benchmark antibody MASP-2-BM exhibited different binding property to truncated proteins and retained some binding to human MASP-2 that lacked the protease domain, confirming that humanized 3E10 and MASP-2-BM bind to different epitopes on MASP-2.

### 6. Reference to Sequence Listing Submitted Electronically

This application incorporates by reference a Sequence Listing with this application as an xml file entitled “[P23400663C].SEQ.xml” created on Mar. 18, 2022 and having a size of 204,816 bytes.

What is claimed is:

1. An antibody or antigen-binding fragment thereof that specifically binds human MASP-2, comprising:
(1) as defined by Kabat,
(a) a light chain variable region (VL) comprising VL CDR1, VL CDR2, VL CDR3 having the amino acid sequences of SEQ ID NOs: 7, 9, and 11, respectively; or a variant thereof having up to about 5 amino acid substitutions, additions, and/or deletions in the VL CDRs; and/or
(b) a heavy chain variable region (VH) comprising VH CDR1, VH CDR2, VH CDR3 having the amino acid sequences of SEQ ID NOs: 12, 16, and 18, respectively; or a variant thereof having up to about 5 amino acid substitutions, additions, and/or deletions in the VH CDRs; or
(2) as defined by IMGT,
(a) a VL comprising VL CDR1, VL CDR2, VL CDR3 having the amino acid sequences of SEQ ID NOs: 8, 10, and 11, respectively; or a variant thereof having up to about 5 amino acid substitutions, additions, and/or deletions in the VL CDRs; and/or
(b) a VH comprising VH CDR1, VH CDR2, VH CDR3 having the amino acid sequences of SEQ ID NOs: 13, 17, and 19, respectively; or a variant thereof having up to about 5 amino acid substitutions, additions, and/or deletions in the VH CDRs.

2. The antibody or antigen-binding fragment of claim 1, comprising VL CDR1, VL CDR2, VL CDR3, VH CDR1, VH CDR2 and VH CDR3 having the amino acid sequences of SEQ ID NOs: 7, 9, 11, 12, 16 and 18, respectively, as defined by Kabat.

3. The antibody or antigen-binding fragment of claim 1, comprising VL CDR1 having the amino acid sequence of SEQ ID NO: 8; VL CDR2 having the amino acid sequence of SEQ ID NO: 10; VL CDR3 having the amino acid sequence SEQ ID NO: 11; VH CDR1 having the amino acid sequence of SEQ ID NO: 13, 14, or 15; VH CDR2 having the amino acid sequence of SEQ ID NO: 17; and VH CDR3 having the amino acid sequences of SEQ ID NO: 19 or 20; as defined by IMGT.

4. The antibody or antigen-binding fragment of claim 3, comprising VL CDR1, VL CDR2, VL CDR3, VH CDR1, VH CDR2 and VH CDR3 having the amino acid sequences of:
(1) SEQ ID NOs: 8, 10, 11, 13, 17, and 19, respectively;
(2) SEQ ID NOs: 8, 10, 11, 14, 17, and 19, respectively;
(3) SEQ ID NOs: 8, 10, 11, 15, 17, and 19, respectively;
(4) SEQ ID NOs: 8, 10, 11, 13, 17, and 20, respectively;
(5) SEQ ID NOs: 8, 10, 11, 14, 17, and 20, respectively; or,
(6) SEQ ID NOs: 8, 10, 11, 15, 17, and 20, respectively.

5. An antibody or antigen-binding fragment thereof that specifically binds human MASP-2, comprising:
(a) a VL having at least 85%, at least 90%, at least 95%, at least 98%, or 100% sequence identity to the amino acid sequence of SEQ ID NO: 21; and/or
(b) a VH having at least 85%, at least 90%, at least 95%, at least 98%, or 100% sequence identity to the amino acid sequence of SEQ ID NO: 22.

6. The antibody or antigen-binding fragment of claim 5 comprising a VL and a VH having the amino acid sequences of SEQ ID NOs: 21 and 22, respectively.

7. An antibody or antigen-binding fragment thereof that specifically binds human MASP-2, comprising
(a) a VL comprising VL CDR1, VL CDR2, and VL CDR3 from a VL having the amino acid sequence of SEQ ID NO: 21; and/or
(b) a VH comprising VH CDR1, VH CDR2, and VH CDR3 from a VH having the amino acid sequence of SEQ ID NO: 22.

8. The antibody or antigen-binding fragment of any one of claims 1 to 7 that is a chimeric antibody or antigen-binding fragment, a humanized antibody or antigen-binding fragment, or a human antibody or antigen-binding fragment.

9. The antibody or antigen-binding fragment of claim 8 that is a humanized antibody or antigen-binding fragment.

10. The antibody or antigen-binding fragment of claim 9, comprising:
(a) a VL having at least 85%, at least 90%, at least 95%, at least 98%, or 100% sequence identity to the amino acid sequence of SEQ ID NO: 23; and/or
(b) a VH having at least 85%, at least 90%, at least 95%, at least 98%, or 100% sequence identity to the amino acid sequence of SEQ ID NO: 24.

11. The antibody or antigen-binding fragment of claim 10 comprising a VL having the amino acid sequence of SEQ ID NO: 23 and a VH having an amino acid sequence selected from the group consisting of SEQ ID NOs: 24-32.

12. The antibody or antigen-binding fragment of any one of claims 1 to 11 that is selected from the group consisting of a Fab, a Fab′, a F(ab′)₂, a Fv, a scFv, a (scFv)₂, a single domain antibody (sdAb), and a heavy chain antibody (HCAb).

13. The antibody or antigen-binding fragment of any one of claims 1 to 11 that is an IgG1 antibody or a variant thereof, an IgG2 antibody or a variant thereof, an IgG3 antibody or a variant thereof, or an IgG4 antibody or a variant thereof.

14. The antibody of claim 13 that is an IgG4 antibody or a variant thereof.

15. The antibody of claim 14 comprising
(1) a light chain having at least 85%, at least 90%, at least 95%, at least 98%, or 100% sequence identity to the amino acid sequence of SEQ ID NO: 33; and
(2) a heavy chain having at least 85%, at least 90%, at least 95%, at least 98%, or 100% sequence identity to an amino acid sequence selected from the group consisting of SEQ ID NO: 34.

16. The antibody of claim 15, wherein the heavy chain has an amino acid sequence selected from the group consisting of SEQ ID NOs: 34-42, or a variant thereof modified by one or more amino acid substitution(s) that increases the antibody's terminal half-life.

17. The antibody of claim 16, wherein the heavy chain variant is modified by one or more substitution(s) at an amino acid residue selected from the group consisting of S228, F234, L235, M252, S254, T256, K288, T307, M428, N434, H435, and Y436 (numbered according to the EU Index).

18. The antibody of claim 16, wherein the heavy chain variant is modified by amino acid substitutions selected from the group consisting of i) S228P; ii) F234A and L235A; iii) S228P, F234A, and L235A; (iv) T307H and N434A; v) M252Y, S254T and T256E; vi) M428L, N434A and Y436T; vii) S228P, M252Y, S254T and T256E; viii) S228P, F234A, L235A, M252Y, S254T and T256E; ix) S228P, F234A, L235A, T307Q, and N434A; and (x) M252Y, S254T, T307H and N434A.

19. The antibody of claim 15, wherein the light chain has the amino acid sequence of SEQ ID NO: 33 and the heavy chain has an amino acid sequence selected from the group consisting of SEQ ID NOs: 43-74.

20. An antibody or antigen-binding fragment thereof that competes with the antibody or antigen-binding fragment of any one of claims 1 to 19 for binding to human MASP-2.

21. The antibody or antigen-binding fragment of any one of claims 1 to 20 that is a bispecific antibody or a multispecific antibody.

22. The antibody or antigen-binding fragment of any one of claims 1 to 21 that is a monoclonal antibody or antigen-binding fragment.

23. The antibody or antigen-binding fragment of any one of claims 1 to 22, wherein the antibody or antigen binding fragment:
(1) binds human MASP-2 with a K_{D} that is about 1 nM or less, measured by surface plasmon resonance (SPR);
(2) blocks C4 activation with an IC50 of about 0.1 μg/mL or less, measured in vitro;
(3) blocks C3 activation with an IC50 of about 0.1 μg/mL or less, measured in vitro;
(4) blocks MAC activation with an IC50 of about 0.1 μg/mL or less, measured in vitro;
(5) does not affect the classical or alternative pathway of completement activation; or
(6) any combination of (1)-(5).

24. An antibody or antigen-binding fragment thereof that specifically binds the protease domain of human MASP-2, wherein the antibody or antigen binding fragment:
(1) binds human MASP-2 with a K_{D} that is 1 nM or less, measured by SPR;
(2) blocks C4 activation with an IC50 of 0.1 μg/mL or less, measured in vitro;
(3) blocks C3 activation with an IC50 of 0.1 μg/mL or less, measured in vitro;
(4) blocks MAC activation with an IC50 of 0.1 μg/mL or less, measured in vitro;
(5) does not affect the classical or alternative pathway of completement activation; or
(6) any combination of (1)-(5).

25. The antibody or antigen-binding fragment of claim 23 or 24 that blocks C4 activation with an IC50 of 0.001-0.01 μg/mL, measured in vitro.

26. The antibody or antigen-binding fragment of claim 23 or 24 that blocks C4 activation in the presence of 5-50% human serum with an IC50 of 0.1 μg/mL or less, measured in vitro.

27. The antibody or antigen-binding fragment of any one of claims 23 to 26, that binds human MASP-2 with a K_{D} that ranges from 0.01 nM to 1 nM as measured by SPR.

28. The antibody or antigen-binding fragment of claim 27, that binds human MASP-2 with a K_{D} that ranges from 0.05 nM to 0.5 nM as measured by SPR.

29. A polynucleotide encoding the antibody or antigen-binding fragment of any one of claims 1 to 28.

30. A vector comprising the polynucleotide of claim 29.

31. A host cell comprising the polynucleotide of claim 29, or the vector of claim 30.

32. A pharmaceutical composition comprising a therapeutically effective amount of the antibody or antigen-binding fragment of any one of claims 1 to 28, and a pharmaceutically acceptable carrier.

33. A method of inhibiting MASP-2-dependent complement activation in a subject in need thereof, comprising administering to the subject an effective amount of the antibody or antigen-binding fragment of any one of claims 1 to 28.

34. The method of claim 33, wherein the subject has a disease or disorder associated with MASP-2-dependent complement activation.

35. A method of treating a disease or disorder associated with MASP-2-dependent complement activation in a subject in need thereof, comprising administering to the subject a therapeutically effective amount of the antibody or antigen-binding fragment of any one of claims 1 to 28.

36. The method of claim 35, further comprising administering an additional therapy to the subject.

37. The method of any one of claims 33 to 36, wherein the subject is a human.

38. Use of the antibody or antigen-binding fragment of any one of claims 1 to 28 in treating a disease or disorder associated with MASP-2-dependent complement activation.

39. Use of the antibody or antigen-binding fragment of any one of claims 1 to 28 for the preparation of a medicament for treating a disease or disorder associated with MASP-2-dependent complement activation.

40. The method or use of any one of claims 34 to 39, wherein the disease or disorder is a renal disease or disorder, a vascular disease or disorder, a skin disease or disorder, an ophthalmologic disease or disorder, a nervous system disease or disorder, a blood disease or disorder, a musculoskeletal disease or disorder, a urogenital disease or disorder, a metabolic disease or disorder, an endocrine disease or disorder, a gastrointestinal disease or disorder, or a pulmonary disease or disorder.

41. The method or use of any one of claims 34 to 39, wherein the disease or disorder is IgA nephropathy (IgAN), thrombotic microangiopathy (TMA), lupus nephritis (LN), membranous nephropathy (MN), or C3 glomerulopathy (C3G).

42. The method or use of claim 41, wherein the disease or disorder is IgAN.

43. The method or use of claim 41, wherein the disease or disorder is TMA.

44. The method or use of claim 43, wherein the disease or disorder is atypical hemolytic uremic syndrome (aHUS), TMA associated with hematopoietic stem cell transplantation (HSCT-TMA), thrombotic thrombocytopenia purpura (TTP), TMA secondary to cancer, TMA secondary to chemotherapy, or TMA secondary to transplantation.

45. The method or use of claim 44, wherein the disease or disorder is HSCT-TMA.

46. The method or use of claim 44, wherein the disease or disorder is TTP.


SEQUENCE LISTING (79 sequences)

SEQ ID NO: 1 - protein, 671 aa, synthetic construct
TPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSALCSGQVFTQRSGELSSPEYPRPYPKLSSCTYSISLEEGFSVILDFVESFDVETHPETLCPYDFLKIQTDREEHGPFCGKTLPHRIETKSNTVTITFVTDESGDHTGWKIHYTSTAQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 2 - protein, 414 aa, synthetic construct
TPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSALCSGQVFTQRSGELSSPEYPRPYPKLSSCTYSISLEEGFSVILDFVESFDVETHPETLCPYDFLKIQTDREEHGPFCGKTLPHRIETKSNTVTITFVTDESGDHTGWKIHYTSTAQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPV

SEQ ID NO: 3 - protein, 345 aa, synthetic construct
TPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSALCSGQVFTQRSGELSSPEYPRPYPKLSSCTYSISLEEGFSVILDFVESFDVETHPETLCPYDFLKIQTDREEHGPFCGKTLPHRIETKSNTVTITFVTDESGDHTGWKIHYTSTAQPCPYPMAPPNGHVSPVQAKYILKDSFSIFCETGYELLQGHLPLKSFTAVCQKDGSWDRPMPA

SEQ ID NO: 4 - protein, 279 aa, synthetic construct
TPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSALCSGQVFTQRSGELSSPEYPRPYPKLSSCTYSISLEEGFSVILDFVESFDVETHPETLCPYDFLKIQTDREEHGPFCGKTLPHRIETKSNTVTITFVTDESGDHTGWKIHYT

SEQ ID NO: 5 - protein, 166 aa, synthetic construct
TPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCS

SEQ ID NO: 6 - protein, 122 aa, synthetic construct
TPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAE

SEQ ID NO: 7 - protein, 17 aa, synthetic construct
KSSQNILYSSNQKNLLA

SEQ ID NO: 8 - protein, 12 aa, synthetic construct
QNILYSSNQKNL

SEQ ID NO: 9 - protein, 7 aa, synthetic construct
WASTRES

SEQ ID NO: 10 - skipped

SEQ ID NO: 11 - protein, 8 aa, synthetic construct
HQYFSSYT

SEQ ID NO: 12 - protein, 5 aa, synthetic construct
TYGVH

SEQ ID NO: 13 - protein, 8 aa, synthetic construct
GFSLTTYG

SEQ ID NO: 14 - protein, 8 aa, synthetic construct
GFTFSTYG

SEQ ID NO: 15 - protein, 8 aa, synthetic construct
GFTLSTYG

SEQ ID NO: 16 - protein, 16 aa, synthetic construct
VIWKGGSTDYNAAFMS

SEQ ID NO: 17 - protein, 7 aa, synthetic construct
IWKGGST

SEQ ID NO: 18 - protein, 14 aa, synthetic construct
TLELRRRDYYAMDY

SEQ ID NO: 19 - protein, 16 aa, synthetic construct
VKTLELRRRDYYAMDY

SEQ ID NO: 20 - protein, 16 aa, synthetic construct
AKTLELRRRDYYAMDY

SEQ ID NO: 21 - protein, 112 aa, synthetic construct
DIVMTQTTSSLAVSAGEKVTMSCKSSQNILYSSNQKNLLAWYQQKPGQSPKLLIYWASTRESGVPNRFTGSGSGTDFSLTINSVQPEDLAVYYCHQYFSSYTFGGGTKLEMK

SEQ ID NO: 22 - protein, 122 aa, synthetic construct
EVQLQQSGPGLVQSSQSLSITCTVSGFSLTTYGVHWVRLSPGKGLEWLGVIWKGGSTDYNAAFMSRLSITKDNSKSEVFFQMNSLQGDDSAIYYCVKTLELRRRDYYAMDYWGQGTSVTVSS

SEQ ID NO: 23 - protein, 112 aa, synthetic construct
DIVMTQSPDSLAVSLGERATINCKSSQNILYSSNQKNLLAWYQQKPGQPPKLLIYWASTRESGVPDRFSGSGSGTDFTLTISSLQAEDVAVYYCHQYFSSYTFGQGTKLEIK

SEQ ID NO: 24 - protein, 122 aa, synthetic construct
QVQLVESGGGVVQPGRSLRLSCAASGFTFSTYGVHWVRQAPGKGLEWVAVIWKGGSTDYNAAFMSRFTISRDNSKNTIYIQMNSLRAEDTAVYYCAKTLELRRRDYYAMDYWGQGTLVTVSS

SEQ ID NO: 25 - protein, 122 aa, synthetic construct
QVQLVESGGGVVQPGRSLRLSCAASGFTLSTYGVHWVRQAPGKGLEWLGVIWKGGSTDYNAAFMSRLTISKDNSKNTVYIQMNSLRAEDTAVYYCAKTLELRRRDYYAMDYWGQGTLVTVSS

SEQ ID NO: 26 - protein, 122 aa, synthetic construct
QVQLVESGGGVVQPGRSLRLSCAASGFTLSTYGVHWVRQAPGKGLEWLGVIWKGGSTDYNAAFMSRLTISKDNSKSTVYIQMNSLRAEDTAVYYCAKTLELRRRDYYAMDYWGQGTLVTVSS

SEQ ID NO: 27 - protein, 122 aa, synthetic construct
QVQLVESGGGVVQPGRSLRLSCAASGFTLSTYGVHWVRQAPGKGLEWLGVIWKGGSTDYNAAFMSRLTISKDNSKNTVYIQMNSLRAEDTAVYYCVKTLELRRRDYYAMDYWGQGTLVTVSS

SEQ ID NO: 28 - protein, 122 aa, synthetic construct
QVQLVESGGGVVQPGRSLRLSCAASGFSLTTYGVHWVRQAPGKGLEWLGVIWKGGSTDYNAAFMSRLTISKDNSKNTVYIQMNSLRAEDTAVYYCAKTLELRRRDYYAMDYWGQGTLVTVSS

SEQ ID NO: 29 - protein, 122 aa, synthetic construct
QVQLVESGGGVVQPGRSLRLSCAASGFSLTTYGVHWVRQAPGKGLEWLGVIWKGGSTDYNAAFMSRLTISKDNSKSTVYIQMNSLRAEDTAVYYCAKTLELRRRDYYAMDYWGQGTLVTVSS

SEQ ID NO: 30 - protein, 122 aa, synthetic construct
QVQLVESGGGVVQPGRSLRLSCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSS

SEQ ID NO: 31 - protein, 122 aa, synthetic construct
QVQLVESGGGVVQPGRSLRISCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSS

SEQ ID NO: 32 - protein, 122 aa, synthetic construct
QVQLVESGGGVVQPGRSLRISCAVSGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSS

SEQ ID NO: 33 - protein, 219 aa, synthetic construct
DIVMTQSPDSLAVSLGERATINCKSSQNILYSSNQKNLLAWYQQKPGQPPKLLIYWASTRESGVPDRFSGSGSGTDFTLTISSLQAEDVAVYYCHQYFSSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 449 aa, synthetic construct
QVQLVESGGGVVQPGRSLRLSCAASGFTFSTYGVHWVRQAPGKGLEWVAVIWKGGSTDYNAAFMSRFTISRDNSKNTIYIQMNSLRAEDTAVYYCAKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 449 aa, synthetic construct
QVQLVESGGGVVQPGRSLRLSCAASGFTLSTYGVHWVRQAPGKGLEWLGVIWKGGSTDYNAAFMSRLTISKDNSKNTVYIQMNSLRAEDTAVYYCAKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 449 aa, synthetic construct
QVQLVESGGGVVQPGRSLRLSCAASGFTLSTYGVHWVRQAPGKGLEWLGVIWKGGSTDYNAAFMSRLTISKDNSKSTVYIQMNSLRAEDTAVYYCAKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 449 aa, synthetic construct
QVQLVESGGGVVQPGRSLRLSCAASGFTLSTYGVHWVRQAPGKGLEWLGVIWKGGSTDYNAAFMSRLTISKDNSKNTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 449 aa, synthetic construct
QVQLVESGGGVVQPGRSLRLSCAASGFSLTTYGVHWVRQAPGKGLEWLGVIWKGGSTDYNAAFMSRLTISKDNSKNTVYIQMNSLRAEDTAVYYCAKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 449 aa, synthetic construct
QVQLVESGGGVVQPGRSLRLSCAASGFSLTTYGVHWVRQAPGKGLEWLGVIWKGGSTDYNAAFMSRLTISKDNSKSTVYIQMNSLRAEDTAVYYCAKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 449 aa, synthetic construct
QVQLVESGGGVVQPGRSLRLSCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 449 aa, synthetic construct
QVQLVESGGGVVQPGRSLRISCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 449 aa, synthetic construct
QVQLVESGGGVVQPGRSLRISCAVSGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 449 aa, synthetic construct
QVQLVESGGGVVQPGRSLRLSCAASGFTLSTYGVHWVRQAPGKGLEWLGVIWKGGSTDYNAAFMSRLTISKDNSKNTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 449 aa, synthetic construct
QVQLVESGGGVVQPGRSLRLSCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 449 aa, synthetic construct
QVQLVESGGGVVQPGRSLRISCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 449 aa, synthetic construct
QVQLVESGGGVVQPGRSLRISCAVSGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 449 aa, synthetic construct
QVQLVESGGGVVQPGRSLRLSCAASGFTLSTYGVHWVRQAPGKGLEWLGVIWKGGSTDYNAAFMSRLTISKDNSKNTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLHVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 48 - protein, 449 aa, synthetic construct
QVQLVESGGGVVQPGRSLRLSCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLHVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 49 - protein, 449 aa, synthetic construct
QVQLVESGGGVVQPGRSLRISCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLHVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 50 - protein, 449 aa, synthetic construct
QVQLVESGGGVVQPGRSLRISCAVSGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLHVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 51 - protein, 449 aa, synthetic construct
QVQLVESGGGVVQPGRSLRLSCAASGFTLSTYGVHWVRQAPGKGLEWLGVIWKGGSTDYNAAFMSRLTISKDNSKNTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 52 - protein, 449 aa, synthetic construct
QVQLVESGGGVVQPGRSLRLSCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - protein, 449 aa, synthetic construct
QVQLVESGGGVVQPGRSLRISCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 449 aa, synthetic construct
QVQLVESGGGVVQPGRSLRISCAVSGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 449 aa, synthetic construct
QVQLVESGGGVVQPGRSLRLSCAASGFTLSTYGVHWVRQAPGKGLEWLGVIWKGGSTDYNAAFMSRLTISKDNSKNTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHTTQKSLSLSLGK

SEQ ID NO: 56 - protein, 449 aa, synthetic construct
QVQLVESGGGVVQPGRSLRLSCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHTTQKSLSLSLGK

SEQ ID NO: 57 - protein, 449 aa, synthetic construct
QVQLVESGGGVVQPGRSLRISCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHTTQKSLSLSLGK

SEQ ID NO: 58 - protein, 449 aa, synthetic construct
QVQLVESGGGVVQPGRSLRISCAVSGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHTTQKSLSLSLGK

SEQ ID NO: 59 - protein, 449 aa, synthetic construct
QVQLVESGGGVVQPGRSLRLSCAASGFTLSTYGVHWVRQAPGKGLEWLGVIWKGGSTDYNAAFMSRLTISKDNSKNTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 449 aa, synthetic construct
QVQLVESGGGVVQPGRSLRLSCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 449 aa, synthetic construct
QVQLVESGGGVVQPGRSLRISCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 449 aa, synthetic construct
QVQLVESGGGVVQPGRSLRISCAVSGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 449 aa, synthetic construct
QVQLVESGGGVVQPGRSLRLSCAASGFTLSTYGVHWVRQAPGKGLEWLGVIWKGGSTDYNAAFMSRLTISKDNSKNTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 449 aa, synthetic construct
QVQLVESGGGVVQPGRSLRLSCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 449 aa, synthetic construct
QVQLVESGGGVVQPGRSLRISCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 66 - protein, 449 aa, synthetic construct
QVQLVESGGGVVQPGRSLRISCAVSGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - protein, 449 aa, synthetic construct
QVQLVESGGGVVQPGRSLRLSCAASGFTLSTYGVHWVRQAPGKGLEWLGVIWKGGSTDYNAAFMSRLTISKDNSKNTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLQVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 68 - protein, 449 aa, synthetic construct
QVQLVESGGGVVQPGRSLRLSCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLQVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 69 - protein, 449 aa, synthetic construct
QVQLVESGGGVVQPGRSLRISCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLQVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 70 - protein, 449 aa, synthetic construct
QVQLVESGGGVVQPGRSLRISCAVSGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLQVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 71 - protein, 449 aa, synthetic construct
QVQLVESGGGVVQPGRSLRLSCAASGFTLSTYGVHWVRQAPGKGLEWLGVIWKGGSTDYNAAFMSRLTISKDNSKNTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITRTPEVTCVVVDVSQEDPEVQFNWYVDGVEVHNAKTKPREEQFNSTYRVVSVLHVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 72 - protein, 449 aa, synthetic construct
QVQLVESGGGVVQPGRSLRLSCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITRTPEVTCVVVDVSQEDPEVQFNWYVDGVEVHNAKTKPREEQFNSTYRVVSVLHVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 73 - protein, 449 aa, synthetic construct
QVQLVESGGGVVQPGRSLRISCAASGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITRTPEVTCVVVDVSQEDPEVQFNWYVDGVEVHNAKTKPREEQFNSTYRVVSVLHVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 74 - protein, 449 aa, synthetic construct
QVQLVESGGGVVQPGRSLRISCAVSGFSLTTYGVHWVRQAPGKGLEWLGVIWKGGSTDYNAAFMSRLTISKDNSKSTVYIQMNSLRAEDTAVYYCVKTLELRRRDYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITRTPEVTCVVVDVSQEDPEVQFNWYVDGVEVHNAKTKPREEQFNSTYRVVSVLHVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 75 - protein, 212 aa, synthetic construct
QPVLTQPPSLSVSPGQTASITCSGEKLGDKYAYWYQQKPGQSPVLVMYQDKQRPSGIPERFSGSNSGNTATLTISGTQAMDEADYYCQAWDSSTAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 76 - protein, 445 aa, synthetic construct
QVTLKESGPVLVKPTETLTLTCTVSGFSLSRGKMGVSWIRQPPGKALEWLAHIFSSDEKSYRTSLKSRLTISKDTSKNQVVLTMTNMDPVDTATYYCARIRRGGIDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 5 aa, synthetic construct
  REPEAT 1..5: note = (GGGGS)n, n=1, 2, 3, 4, or 5
GGGGS

SEQ ID NO: 78 - protein, 5 aa, synthetic construct
  REPEAT 1..5: note = (EAAAK)n, n=1, 2, 3, 4, or 5
EAAAK

SEQ ID NO: 79 - skipped